(19) United States
(12) Patent Application Publication
(10) Pub. No.: US 2017/0226330 A1
(43) Pub. Date: Aug. 10, 2017

(54) FLUID COMPOSITION COMPRISING LIGNIN

(71) Applicant: Inbicon A/S, Fredericia (DK)

(72) Inventors: Niels Ole Knudsen, Brønderslev (DK); Mia Frosch Mogensbæk Foverskov, Herlev (DK)

(73) Assignee: Inbicon A/S, Fredericia (DK)

(21) Appl. No.: 15/503,490

(22) PCT Filed: Aug. 14, 2015
(86) PCT No.: PCT/DK2015/050242

§ 371 Date: Feb. 13, 2017

Related U.S. Application Data
(60) Provisional application No. 62/037,141, filed on Aug. 14, 2014.

(51) Int. Cl. C08L 5/14; C12P 19/14; C12P 19/04; C08L 1/02; C10L 1/02

(52) U.S. Cl. CPC C08L 5/14; C08L 1/02; C10L 1/02; C12P 19/04; C12P 19/14; C10L 2200/0469; C10L 2270/04; C10L 2290/26; C10L 2290/24; C10L 2290/06; C10L 2290/28; C10L 2290/146; C10L 2290/141; C10L 2290/543; C10L 2290/547; C10L 2290/545; C10L 2290/544; C12P 2201/00

(57) ABSTRACT

The present invention relates to a fluid composition comprising a solid fraction and a liquid organic fraction, wherein said solid fraction and said liquid fraction are present in a state of being intermixed, wherein said solid fraction comprises a lignin component, wherein said liquid fraction comprises an organic substance. Furthermore, the present invention relates to a process for the manufacture of such fluid compositions, to various uses thereof, and to a process for treatment of a lignocellulosic biomass.

### FIELD OF THE INVENTION

The present invention relates generally to fluid compositions comprising a lignin component intermixed with a liquid organic fraction, such as suspended in oil, use of such fluid compositions e.g. as fuel, as well as methods and processes related to said fluid compositions, including their manufacture.

### BACKGROUND OF THE INVENTION

Considerable interest has arisen in liquid fuel compositions derived from lignin. A variety of approaches have been reported whereby solid lignin is converted to liquid fuel by pyrolysis or other thermochemical techniques. However, these technologies are capital intensive.

Some authors have previously reported a simple means for converting solid lignin to liquid fuel by including solid lignin particles as a component of a lignin-oil-water emulsion. See Posarac and Watkinson (2000); Thammachote, N. (2000); U.S. Pat. No. 5,478,366 “Pumpable lignin fuel.”

This simple lignin fuel compositions were never used commercially, in part because they suffered from peculiar viscosity characteristics. These suspensions were only pumpable, that is, able to be transported conveniently to a burner, under conditions of constant agitation. After agitation is discontinued, these suspensions quickly return to a semi-solid, paste-like state. In actual commercial applications for liquid fuels of this sort, it is clearly disadvantageous that high shear mixing be maintained continuously in a fuel storage tank. This requires expensive equipment as well as an increased operating expense.

We have discovered that the problems encountered with this simple lignin emulsion/suspension of the prior art can be solved by using a source of lignin which has lignin ion exchange capacity less than 0.4 mol/kg, or 0.3 mol/kg. In that case, the viscosity of the suspension is generally reduced, such as to within a range that can be maintained in a storage tank using only gentle agitation, for example, with a recirculation pump.

Lignin is a complex phenolic polymer which forms an integral part of the secondary cell walls of various plants. It is believed that lignin is one of the most abundant organic polymers on earth, exceeded only by cellulose, and constituting from 25 to 33% of the dry mass of wood and 20 to 25% for annual crops.

Lignin serves as a strengthening support structure within the plant structure itself in that lignin fills the space in the cell wall between cellulose, hemicellulose and pectin components. Lignin is covalently linked to hemicellulose and therefore crosslinks different plant polysaccharides, conferring mechanical strength to the cell wall by extension in the plant as a whole.

Three major monolignols are involved as phytochemicals in the biosynthesis of lignin. These three monolignols are coniferyl alcohol, sinapyl alcohol and p-coumaryl alcohol.

Lignin originating from different plants exhibit varying mutual contents of these three phytochemicals and accordingly, depending on plant species, lignin appears in nature in a high diversity of different structures.

Traditionally, lignin has been obtained and isolated as a byproduct in the paper manufacturing industry. Accordingly, in the Kraft process, wood chips are cooked in a pressurized digester in a strong alkaline liquid containing sulfide at 130-180° C. Under these conditions lignin and hemicellulose degrade into fragments that are soluble in the alkaline liquid. The cellulose remains solid and is separated off for further paper making processing, whereas the liquid containing the lignin fragments, denoted black liquor, is evaporated to a dry matter content of approximately 65-80%. This concentrated black liquor comprising lignin fragments is burned in order to recover chemicals, such as sodium hydroxide and inorganic sulfur compounds for reuse in the Kraft process and in order to utilize the heat value of the lignin fragments contained in the black liquor.

Lignin is usually not isolated in the Kraft process, but the corresponding lignin fragments are burned in a wet state. However, if the alkaline black liquor is neutralized or acidified with acid, the lignin fragments will precipitate as a solid and may be isolated. A Kraft processing plant may have facilities for isolating the lignin fragments in this way.

Conveniently, the lignin fragments are isolated by solubilizing carbon dioxide, recovered elsewhere in the Kraft process, in the black liquor in order to neutralize/acidify the black liquor resulting in the precipitation of the lignin fragments.

The lignin fragments recovered from the Kraft process have strongly reduced molecular size, and a very high purity compared to the lignin located in the wood chips from which the lignin originates. This reduction of molecular size is due to the fact that the pressurized cooking in the alkaline liquid, takes place in presence of sulfide (S²⁻) or bisulfide (HS⁻) ions, which act as ether bond cleaving reagents, thus cleaving the ether bonds of the lignin and resulting in lignin fragments having strongly reduced sizes. The high purity is due to the fact that Kraft lignin and hemicellulose has been totally solubilized during the cooking process, whereby it has been completely separated from the cellulose fraction, and afterwards only lignin precipitates during acidification.

The energy content (HHV) of Kraft lignin is on average approximately 26 MJ/kg dry matter, slightly below that of coal, and hence lignin represents an interesting alternative source of energy.

Although lignin fragments may be recovered from the black liquor of the Kraft process and may be pelletized and used as a solid fuel for heating, such usage have not been very widespread.

One reason for this may be that solid fuel generally requires specially adapted equipment for loading, feeding and dosing to the boiler in which the pellets are to be burned. Furthermore, as for all kinds of solid fuel, such specially adapted equipment for loading, feeding and dosing of the lignin pellets usually are sensitive as to the pellet size and other fuel qualities, meaning that once a particular fuel having certain specifications has been selected, one cannot easily alter the kind of solid fuel to another kind having another pellet size, thereby imposing restrictions on the flexibility of such solid fuels.

Accordingly, attempts have been made in converting solid lignin derivatives or fragments to liquid fuel formulations.

WO96/10067 A1 discloses a liquid fuel based on a lignin oil slurry. The lignin oil slurry of WO96/10067 is having specified rheological properties thus making it suitable for being poured or pumped. The lignin oil slurry comprises a lignin fragment in an amount of 35-60%; water in an amount of 35-60%, and oil in an amount of 0.5-30%. Furthermore, the slurry comprises a dispersion agent in an amount of at least 50 ppm. The viscosity of the slurry after stirring is 100-1,000 mPa·s. According to the description of WO96/10067, the above stated limits of the amounts of the constituents are essential for the slurry to have the desired properties. The lignin fragment is in the description of WO96/10067 and its examples disclosed as being a lignin fragment originating from a Kraft process.

Another reason for the limited use of Kraft lignin based oil slurries is the limited storage stability of the dispersion related to both the sedimentation rate of the dispersion and risk of microbial contamination of the product.

US 2011/0239973 discloses a fuel mixture for a combustion engine. The fuel mixture comprises a combustible solid powder and a liquid fuel. The combustible solid powder may be selected from the group comprising lignin, nitrification products of biomass and total biomass powder or any combination thereof. The liquid fuel may be selected from gasoline, kerosene, diesel, heavy oil, emulsified heavy oil, absolute ethanol or any combinations thereof.

US2011/0239973 mentions a range of various forms of lignin to be used in that invention. However, the only lignin fragments exemplified in the working embodiments seems to originate from alkali lignin which according to US2011/0239973 is a lignin fragment obtained from black liquor in a paper making Kraft process.

Furthermore, the inventor of US2011/0239973 is concerned about avoiding having too large particle sizes of the lignin fragments, as these will clog the oil supply line of the engine in which the oil is to be used. According to US 2011/0239973 lignin particles will in the presence of moisture adhere to each other resulting in increasing particles sizes. For this reason, the lignin is subjected to a condensation stabilization treatment. Such treatment according to US2011/0239973 results in smaller particle sizes having increased mixing stability.

Although the above mentioned liquid formulations of fuels comprising lignin fragments solves the problems associated with the solid lignin fuel pellets, i.e. the requirement of specially adapted equipment for loading, feeding and dosing such a solid fuel, these liquid formulations still represent an unsatisfactorily solution especially due to lack of stability and high viscosity of the produced fuel.

Another source of a lignin component may be the biomass refining industry.

In the last decade much effort has been focused on the goal of making cellulose a source of renewable energy. The recent development of highly effective cellulases together with innovative improvements in the pretreatment processes necessary in order to make the cellulose accessible to the cellulase enzymes used in the process, has removed important obstacles in reaching this goal.

In the second generation bioethanol producing process, or the biomass refining process for short, a lignocellulosic biomasss comprising cellulose, hemicellulose and lignin may be converted to ethanol. The process involves i) a hydrothermal pretreatment of the lignocellulosic biomass for making the cellulose accessible to catalysts in a subsequent step; followed by ii) a hydrolysis of the cellulose for breaking down the cellulose to soluble carbohydrates and finally iii) a fermentation of the soluble carbohydrates to ethanol.

A fiber fraction and a liquid phase are left behind after the hydrolysis has been performed.

The liquid phase obtained after the hydrolysis step comprises soluble carbohydrates useful for fermentation into ethanol. The remaining fraction obtained after the hydrolysis step comprises a lignin component.

The fiber fraction consist mainly of lignin, cellulose, hemicellulose and ash components. Compared to for example Kraft lignin, the lignin from the 2G bio refining industry is a more complex material, where the physio-chemical properties is only sporadically described. The lignin component may be rinsed, washed, filtered and/or pressed in order to obtain lignin in a more purified state. This will however only remove some of the soluble salts and the carbohydrates with short chain lengths. The rinsed, washed, filtered, dried and/or pressed lignin component obtained this way is usually pressed into pellets and used as a solid fuel.

Lignin fragments originating from a Kraft process are reported to contain only insignificant amount of cellulose and hemicellulose, but up to about 1.5-3.0% sulfur; most being present as organically bound sulfur; however also inorganic sulfur compounds are present in the Kraft lignin fraction as well as minute amounts of elemental sulfur.

With the ever increasing worldwide focus on environmentally concerns, it is undesirably to burn Kraft lignin fragments having such a high sulfur content due to the risk of emission of gaseous sulfur compounds, unless the plant for burning such lignin fragments are equipped with a sulfur filtering device, such as a scrubber or the like.

However, such sulfur filtering devices are not present in most smaller sized heating facilities, such as in household oil burners, in district heating plants etc.

In addition to the merely ethical concerns relating to avoid high sulfur emission to the environment due to the burning of lignin fragments originating from a Kraft process, various governments in different states have provided legislation regulating the maximum allowable emission of sulfur compounds to the environment originating from burning fuel.

Such legislation obviously imposes restrictions to the use of Kraft lignin fragments as a fuel in smaller sized heating facilities not equipped with a sulfur filtering device.

There are several reasons that the liquid fuels disclosed in WO96/10067 and in US2011/0239973 are not fully satisfactorily, one is that these fuels have a high sulfur content. Furthermore, the compositions described in these publications tend to be more viscous than desired, thereby reducing the amount of lignin that it is possible to incorporate into the liquid fuel formulation, furthermore the storage stability of the dispersions is less than desired.

Furthermore, in the described fuel formulations in WO96/10067 it is only possible to reduce the viscosity of the fuel by either increasing the amount of dispersant or by reducing the amount of lignin in the fuel.

Hence, there still exists a need for an improved liquid or fluid fuel formulation comprising lignin components which may allow being burned in a fuel burning device or plant not equipped with a sulfur filtering device, and which are having suitable viscosities allowing them to be pumpable.

Furthermore, there is a need for an improved fluid fuel formulation, where lignin of lower purity than the Kraft lignin, can be used.

The above described disadvantages have been overcome with the present invention.

### BRIEF DESCRIPTION OF THE PRESENT INVENTION

In a first aspect, the present invention concerns a fluid compositions comprising a lignin component, an organic fraction in liquid state at 25° C., and optionally water and/or a further agent. Such a fluid composition may comprise (i) 5-60% (w/w) of a lignin component, (ii) 0-95% (w/w) of a liquid organic fraction, (iii) 0-60% (w/w) water, and (iv) 0-5.0% (w/w) of one or more further agents.

In general, these components are intermixed, and the lignin component is different from e.g. Kraft lignin, characterized e.g. by its lower assessed polarity and other related features.

In a second aspect, the present invention relates to processes related to the fluid composition according to the first aspect of the invention, such as methods and processes for the manufacture of a fluid composition according to the first aspect of the present invention. Such a process according to the second aspect may comprise the steps of: (i) providing a fraction, preferably a solid fraction comprising a lignin component; (ii) providing an organic compound to make up at least part of said liquid organic fraction; and (iii) intermixing the fraction provided in step (i) with the organic compound and/or liquid organic fraction provided in step (ii). Again, the lignin component in step (i) is a “non-Kraft” lignin with advantageous features as described herein, including lower LIEC, lower hygroscopy, and/or lower swelling.

In a third aspect, the present invention concerns to processes related to treatment of lignocellulosic biomasses, such as a process for treatment of a lignocellulosic biomass, said process comprising:
a) subjecting said lignocellulosic biomass for hydrothermal pretreatment resulting in a hydrothermally pretreated lignocellulosic biomass;
b) subjecting at least part of said hydrothermally pretreated lignocellulosic biomass obtained in step (a) to a hydrolysis resulting in a liquid fraction comprising soluble carbohydrates, and a fiber fraction comprising a lignin component;
c) optionally subjecting at least part of the liquid fraction obtained in step (b) to a fermentation in order to ferment at least part of said soluble carbohydrates to a fermentation product, such as ethanol, methane or butanol, thereby obtaining a fermentation broth;
d) optionally isolating at least part of said fermentation product from the fermentation broth obtained in step (c) e.g. by distillation;
e) isolating at least part of the lignin component from one or more of: the fiber fraction obtained in step (b); the fermentation broth obtained in step (c); or after isolation of at least a part of the fermentation product in step (d);
f) converting at least part of the lignin component obtained in step (e) to a fluid composition, such as a fluid composition according to the first aspect of the invention, by admixing said lignin component with a liquid organic fraction comprising an organic compound or substance.

In a fourth aspect, the present invention relates to uses of a fluid composition according to the first aspect of the present invention, including a fluid composition provided according to the second and/or third aspect of the present invention. This includes uses of the fluid composition as fuel.

In a fifth aspect, the present invention relate to the use of lignin or a solid lignin component for a fluid composition, such as a fluid composition according to the first, second or third aspect of the present invention. This includes also uses related to chemical processing of lignin and/or a lignin component or a conversion product thereof. The lignin and/or lignin component can e.g. be, or be provided as described in one of the other aspects of the invention.

### BRIEF DESCRIPTIONS OF THE DRAWINGS

FIG. 1a shows the sum of ignition delay and ignition time (left) and pyrolysis time (right) of samples at the conditions: 1200° C., 5.5% O2 (dry) and a flue gas velocity of 1.6 m/s. The full drawn lines are linear trendlines of the datasets.

FIG. 1b shows the sum of ignition delay and ignition time (left) and pyrolysis time (right) of samples at the conditions: 1200° C., 2.9% O2 (dry) and a flue gas velocity of 1.6 m/s. The full drawn lines are linear trendlines of the datasets.

FIG. 1c shows the sum of ignition delay and ignition time (left) and pyrolysis time (right) of samples at the conditions: 990° C., 5.5% O2 (dry) and a flue gas velocity of 1.6 m/s. The full drawn lines are linear trendlines of the datasets.

FIG. 2: Lignin filter cake; before (left) and after being cut up with the Kenwood machine (right)

FIG. 3: Schematic of nonylphenol ethoxylate.

FIG. 4: Viscosity of Lignomulsion (LOW without any additives) at different shear rates and constant temperature (a) and different temperature and shear rates 100 s⁻¹ (b).

FIG. 5: Viscosity of Lignomulsion (LOW with 5000 ppm Lutensol AP10 and 5000 ppm Sodium benzoate) different temperature and shear rate 100 s⁻¹. The oil content in the formulations is either 0% (a), 10% (b), 20% (c) or 30% (d).

FIG. 6: Effect of sodium benzoate and Lutensol AP10 on viscosity; measured as a function of shear rate at room temperature.

FIG. 7: Effect of sodium benzoate and Lutensol AP10 on viscosity; measured as a function of temperature at shear rate 100 s⁻¹.

FIG. 8: Viscosity of LOW 40-20-40 formulations with the different addition; either with 5000 ppm Lutensol AP10 with a hydrotrope (a), or with 5000 ppm sodium benzoate with a surfactant (b).

FIG. 9: Paraben structure

FIG. 10a-b Viscosity as a function of shear rate at room temperature (a) and as a function of temperature at shear rate 100 s⁻¹ (b) for emulsions with composition LOW 40-20-40 and 0.5% Lutensol AP10 and various amounts of methylparaben, propylparaben and sodium benzoate.

FIG. 11: Viscosity of LOW formulations using different oil types, measured at shear rate 100 s⁻¹ and at four different temperatures. The formulations are LOW 40-20-40 (a) and LOW 40-30-30 (b), in both cases with 5000 ppm sodium benzoate and 5000 ppm Lutensol AP10.

FIG. 12: Viscosity of different LOW formulations using unrefined palm oil as a function of shear rate at room temperature (a) and temperature at shear rate 100 s⁻¹ (b). The formulations all contain 5000 ppm sodium benzoate and 5000 ppm Lutensol AP10.

FIG. 13: Viscosity of Lignomulsion with and without heavy fuel oil as a function of shear rate.

FIG. 14: Viscosity of lignomulsions with and without heavy fuel oil as a function of temperature.

FIG. 15: Viscosity of emulsions with various diesel:fuel oil ratios as a function of shear rate.

FIG. 16: Viscosity of Lignomulsion formulations without oil or additives; shown as a function of shear rate measured at room temperature (a); and of temperature measured at shear rate 100 s⁻¹ (b).

FIG. 17: Viscosity of Lignomulsion formulations with 10% diesel oil and without additives; shown as a function of shear rate measured at room temperature (a); and of temperature measured at shear rate 100 s⁻¹ (b).

FIG. 18: Viscosity of Lignomulsion formulations with 10% diesel oil and Lutensol AP10 and sodium benzoate; shown as a function of shear rate measured at room temperature (a); and of temperature measured at shear rate 100 s⁻¹ (b).

FIG. 19: Viscosity of Lignomulsion formulations with different oil content; shown as a function of shear rate measured at room temperature (a); and of temperature measured at shear rate 100 s⁻¹ (b).

FIG. 20: Viscosity of different Lignomulsion formulations with and without additives; shown as a function of shear rate measured at room temperature (a and c); and of temperature measured at shear rate 100 s⁻¹ (b and d).

FIG. 21: Viscosities of different formulations shown as a function of shear rate measured at room temperature.

FIG. 22: Lignomulsion (prepared with Indulin, sample ID 140528_001) less than ten minutes after homogenization.

FIG. 23: Viscosity at room temperature and shear rate 100 s⁻¹ as a function of time (for three different formulations).

FIG. 24: Viscosity of LOW emulsions prepared from lignin filtercake, dried to 99% DM; shown as a function of shear rate measured at room temperature (a) or as a function of temperature measured at shear rate 100 s⁻¹ (b).

FIG. 25: Viscosity of LOW emulsions prepared from lignin filtercake, dried at 50° C.; shown as a function of shear rate measured at room temperature (a) or as a function of temperature measured at shear rate 100 s⁻¹ (b).

FIG. 26: Viscosity as a function of shear rate; first four runs at 85° C. for emulsions with the same formulation prepared either at room temperature or at 85° C.

FIG. 27: Viscosity as a function of shear rate for emulsions with the same formulation prepared either at room temperature or at 85° C.

FIG. 28: Viscosity as a function of shear rate at different temperatures of LOW 30-00-70 Lignomulsion before (a) and after (b) treatment in the Parr reactor

FIG. 29: Viscosity of LOW formulations as a function of shear rate at room temperature (a) and as a function of temperature at shear rate 100 s−1 (b).

FIG. 30: Viscosity of LOW formulations as a function of the Indulin fraction

FIG. 31: Viscosity measured at shear rate 100 s⁻¹ and 25° C. as a function of klason lignin (a) and the sum of glucan and xylan (b).

FIG. 32: Correlation between the content of sugar and klason lignin in 12 lignin samples

FIG. 33: Viscosity at room temperature and shear rate 100 s⁻¹; divided in to group based on pretreatment severity (a) and hydrolysis/fermentation conditions (b)

FIG. 34: Viscosity at room temperature and shear rate 100 s⁻¹ of 12 lignin samples shown as a function of klason lignin content in the lignin sample. As the lignin content in the formulation was 30%, the mass contribution of klason lignin to the entire formulation is actually in the range 12-21% in the LOW 30-20-50 formulations (a) and 16-28% in the LOW 40-10-50 formulations (b).

FIG. 35 Viscosity of Lignomulsion prepared with untreated, acid treated or base treated lignin pellets (grinded).

FIG. 36: Energy consumption of ultra turrax as a function of duration a constant speed of 10000 rpm (a) and as a function of speed at constant durations of 0.5 min and 10 min, respectively (b).

FIG. 37: Emulsion viscosity as a function of energy consumed by the UT at constant speed (10.000 rpm) and duration from 0.5 to 10 min.

FIG. 38: Emulsion viscosity as a function of energy consumed by the UT at constant duration; 0.5 min (a) and 10 min (b). The UT duration varied from 3500 to 20000 rpm.

FIG. 39: Contour plot of viscosity (Pa s) as a function of speed (“stirring”/rpm) and duration/time (min).

FIG. 40: Viscosity of Lignomulsion samples before (1st run) and after (2nd run) storage.

FIG. 41: Size distributions of emulsions containing ISK lignin, prepared directly from filter cake with ultra turrax (exp. 1-6, see table 25-1).

FIG. 42: Size distribution of lignin, milled from dry lignin pellets and separated with different sieves.

FIG. 43: System for injecting lignomulsion into combustion chamber

FIG. 44: Modified injection system where the fuel (lignomulsion) is mixed with pressurized air before the nozzle (full cone).

FIG. 45: Viscosity at shear rate 100 s−1.

### DETAILED DESCRIPTION OF THE INVENTION

The lignin component obtained in the biomass refining process is quite different from the lignin fragments obtained in the Kraft process. First, in the Kraft process, the lignin polymer obtained from the lignocellulosic biomass has been subjected to alkaline liquid containing ether bond cleaving inorganic sulfur species resulting in cleavage of a high number of the ether bonds originally present in the lignin molecules with the consequence that the macromolecular original lignin molecules are cleaved into a larger number of smaller lignin fragments. Secondly, these smaller lignin fragments are solubilized and dissolved in the alkaline liquid in the used in the Kraft process and only re-precipitated upon acidifying the black liquor. Hence, the lignin fragments obtained from a Kraft process comprise relatively small lignin species which have been dissolved in the black liquor and which have a relatively high purity, but also a high sulfur content.

In contrast, it is believed that a lignin component obtained from the biorefining of a lignocellulosic biomass has not been processed in a way that makes it dissolve and/or improve its solubility. Furthermore, such a lignin component has not been subjected to a treatment involving ether bond cleaving reagents which will result in a moderately high increase in sulfur content. Hence, a lignin component obtained from biorefining of a lignocellulosic biomass comprises undissolved lignin, residual hemicellulose, cellulose and ash components. Furthermore the lignin molecules are relatively large and have a relatively low sulfur content.

The present invention according to its various aspects represents great advantages compared to the prior art.

### First Aspect

First of all, the product of the first aspect is a fluid or a liquid which means that the problems associates with lignin pellets used for fuel as described above is eliminated with the present invention.

Secondly, the fluid composition according to the first aspect of the present invention is advantageous in that it contains a relatively low content of sulfur which makes it acceptable for use as a fuel in heat producing plants not provided with a sulfur filtering device for its effluent gases.

Thirdly, the fluid composition according to the first aspect of the present invention is advantageous in that can be produced from low purity lignin components.

Fourthly, the stability of the composition according to the first aspect of the present invention is improved due to the hydrotropic action of the cellulose and hemicellulose.

Fifthly, the viscosity of the composition according to the first aspect of the present invention can be reduced by adding hydrotropic compounds.

In addition, the idea of converting a solid fuel, such as a lignin component, to a liquid fuel is that the handling, storage and transport of liquid fuels are much more convenient, compared to solid fuels. However, in handling storing and transportation of liquid fuels also the viscosity of the liquid plays an important role. A fuel having a low viscosity is much easier to handle than a fuel having a high viscosity.

When making fluid compositions, such as dispersions of lignin in various organic substances, the trend has been observed that the higher the concentration of lignin, the higher the viscosity of the resulting dispersion. There has hitherto been no possibility of lowering the viscosity Unfortunately it has been found that this lowest obtainable limit of viscosity in many liquid formulations of Kraft lignin fragments is undesirably high.

In contrast, when using a lignin obtained from a biorefinery process of a lignocellulosic biomass, involving hydrothermal pretreatment of the biomass followed by a hydrolysis of the biomass, for making a dispersion of a lignin component and an organic substance, much lower viscosities of the dispersion may be encountered. Indeed this is also what the present inventors have found with the fluid composition according to the first aspect of the present invention.

Hence, a wider range of flexibility relating to viscosity of a fluid composition comprising a lignin component may be obtained with fluid composition according to the invention according to the first aspect of the present invention, because starting with a lignin component obtained from a biorefinery process of a lignocellulosic biomass, involving hydrothermal pretreatment of the biomass followed by a hydrolysis of the biomass, for making a dispersion of a lignin component and an organic substance, the initial viscosity will be lower than if Kraft lignin has been used.

Moreover, the lignin component obtained from a biorefinery process of a lignocellulosic biomass, involving hydrothermal pretreatment of the biomass followed by a hydrolysis of the biomass, when used for a fluid composition according to the first aspect of the present invention need not be dried prior to the admixing with the organic substance of the liquid fraction of the fluid, and the lignin component may even be intermixed with this organic substance of the liquid fraction of the fluid in a wet state while still resulting in a stable fluid composition, such as a stable dispersion.

Furthermore, it has surprisingly been found that when using a lignin obtained from a biorefinery process of a lignocellulosic biomass, involving hydrothermal pretreatment of the biomass followed by a hydrolysis of the biomass, for making a dispersion of a lignin component and an organic substance, stable fluids, a more stable dispersion may be obtained due to the content of residual cellulose and hemicellulose even if no dispersion agent is used. Such avoidance of dispersion agents contributes huge savings in the production cost of the fluid composition, not least in case the fluid composition is going to be used as a fuel, where the demand of that specific fuel may be in the order of thousands of metric tons per year.

Furthermore, it has surprisingly been found that when using a lignin obtained from a biorefinery process of a lignocellulosic biomass, the viscosity of the fluid composition can be reduced by adding hydrotropes to the composition, this will result in significant cost savings as the lignin content can be increased and still be able to pump the resulting fluid composition.

Furthermore, it has surprisingly been found that when using a lignin obtained from a biorefinery process of a lignocellulosic biomass, involving hydrothermal pretreatment of the biomass followed by a hydrolysis of the biomass, for making a dispersion of a lignin component and an organic substance, stable fluids, a more stable dispersion may be obtained even if no dispersion agent is used. This is due to the low tendency of such lignin components to interact with water. This tendency is quantified by the Lignin Ion Exchange Capacity (LIEC), describing as the amount of polar groups per lignin weight unit. Such avoidance of dispersion agents contributes huge savings in the production cost of the fluid composition, not least in case the fluid composition is going to be used as a fuel, where the demand of that specific fuel may be in the order of thousands of metric tons per year.

The above advantages are quite surprising and could not have been foreseen by a person skilled in the art.

In the context of the present invention, the term “fluid composition” as used in the present description and in the appended claims shall be understood to mean a composition which is fluid or liquid in the sense that it exhibits viscosities at various temperatures falling within ranges as herein, in particular at room temperature, e.g. at 20 or 25° C.

A “liquid” is meant to comprise a near-incompressible fluid that conforms to the shape of its container but retains a (nearly) constant volume independent of pressure at room temperature, e.g. at 20 or 25° C. Being liquid is one of the four fundamental states of matter other than being solid, gas, or plasma.

Thus, as mentioned above, in a first aspect the present invention relates to a fluid composition comprising: a solid fraction and a liquid fraction; wherein said solid fraction and said liquid fraction are present in a state of being intermixed; wherein said solid fraction comprises a lignin component; and wherein said liquid fraction comprises an organic substance.

The fluid composition of the first aspect of the present invention comprises a solid fraction and a liquid fraction.

The solid fraction of the fluid composition of the first aspect in turn comprises a lignin component often also containing some cellulose and/or hemicellulose.

Whereas the term “lignin” in the present description and in the appended claims refers to the polymer denoted as such and being present in unprocessed lignocellulosic plant material, the term “lignin component” in the present description and in the appended claims has a broader meaning. The term “lignin component” shall the in the present description and in the appended claims be understood to mean a “lignin” that has been subject to various physical and/or chemical treatments imposing minor changes of the lignin polymer structure, however still retaining its polymer character and containing significant amounts of hemicellulose and cellulose.

Hence a “lignin component” as used in the present description and in the appended claims may refer to a lignin that has been subjected to slight structural modifications.

Also a “lignin component” as used in the present description and in the appended claims may refer to a lignin that has been subjected to slight structural modifications and/or comprising an amount of chemical residues originating from its mode of manufacture, or originating from compounds native for the lignocellulosic material from which it is isolated.

In some embodiments of the various aspects of the present invention a “lignin component” may specifically exclude a Kraft lignin or a Kraft lignin fragment obtained from a Kraft processing of a lignocellulosic biomass.

In some embodiments of the various aspects of the present invention a “lignin component” may specifically exclude a lignosulfonate.

In some embodiments of the various aspects of the present invention a “lignin component” may specifically exclude a soda lignin.

In the context of the present invention, the “lignin component” is meant to comprise a by-product from 2^{nd} generation (2G) bioethanol production. There are various different 2^{nd} generation bio-ethanol processes known in the art that may provide such a lignin component, incl. organosolv processes. Schemes for processing lignocellulosic biomass, including specific process steps as well as overall schemes for converting a lignocellulosic biomass to soluble saccharides and a fibrous fraction being or comprising the lignin component, are the subject of numerous published patents and patent applications. See e.g. WO 94/03646; WO 94/29474; WO 2006/007691; US2007/0031918; WO 2008/112291; WO 2008/137639; EP 2 006 354; US 2009/0326286; US 2009/0325251; WO 2009/059149; US 2009/0053770; EP 2 169 074; WO 2009/102256; US 2010/0065128; US 2010/0041119; WO 2010/060050; WO2007009463 A2, WO2007009463 A1; WO2011125056 A1; and WO2009125292 A2, each of which is hereby incorporated by reference in entirety.

According to the first aspect of the present invention, the solid fraction and the liquid fraction of the fluid composition are present in a state of being intermixed. The term “present in a state of being intermixed” shall in the present description and in the appended claims be understood to mean that the solid fraction and the liquid fraction of the fluid composition have been subjected to some kind of mechanical action which have brought them into an intermixed state.

Hence, in this way, the solid fraction and the liquid fraction of the fluid composition of the first aspect of the present invention may be present in a state in which the solid fraction is approximately evenly distributed in the liquid fraction of the fluid composition.

The liquid fraction of the fluid composition according to the first aspect of the present invention comprises an organic substance.

The term “organic substance” shall in the present description and the amended claims be understood to be any substance which in said liquid fraction upholds a liquid character, meaning that said organic substance at various temperature exhibit viscosities as defined below.

In some embodiments of the various aspects of the present invention, the term “organic substance” shall mean a substance which comprises one or more carbon atoms, wherein at least one of said one or more carbon atoms is bonded to adjacent atoms by forming covalent bonds.

In some embodiments of the various aspects of the present invention, the organic substance preferably itself is an organic substance which is capable of participating in an exothermal reaction with oxygen, such as an oil or a fuel.

The first aspect of the invention pertains to a fluid composition comprising a lignin component, an organic fraction in liquid state at 25° C., and optionally water and/or a further agent. Such a fluid composition may comprise, contain, consist, or consist essentially of: (i) a lignin component (“L”), such as 5-60% (w/w); (ii) a liquid organic fraction (“O”), such as 0-60% (w/w), said liquid organic fraction being in a liquid state at room temperature, such as 25° C.; and optionally (iii) water (“W”) in the range of 0-95% (w/w), and optionally (iv) a further agent (“A”). In some embodiments, said further agent is present in a concentration of 1%, or below. In some embodiments, the further agent is present in the range of 0-0.5% (w/w). In many, but not alle some embodiments of the first aspect of the present invention, said liquid fraction of the fluid composition furthermore comprises water.

Usually the composition comprises, contains, consists, consists essentially of a fluid composition with a lignin or lignin component, liquid organic fraction, and water content (“L-O-W”, expressed as %-%-% (w/w)) of, or around:

5-0-95, 5-5-90, 5-10-85, 5-15-80, 5-20-75, 5-25-70, 5-30-65, 5-35-60,
10-0-90, 10-5-85, 10-10-80, 10-15-75, 10-20-70, 10-25-65, 10-30-60,
15-0-85, 15-5-80, 15-10-75, 15-15-70, 15-20-65, 15-25-60, 15-30-55,
20-0-80, 20-5-75, 20-10-70, 20-15-65, 20-20-60, 20-25-55, 20-30-50,
25-0-75, 25-5-70, 25-10-65, 25-15-60, 25-20-55, 25-25-50, 25-30-45,
30-0-70, 30-5-65, 30-10-60, 30-15-55, 30-20-50, 30-25-45, 30-30-40,
35-0-65, 35-5-60, 35-10-55, 35-15-50, 35-20-45, 35-25-40, 35-30-35,
40-0-60, 40-5-55, 40-10-50, 40-15-45, 40-20-40, 40-25-35, 40-30-30,
45-0-55, 45-5-50, 45-10-45, 45-15-40, 45-20-35, 45-25-30, 45-30-25,
50-0-50, 50-5-45, 50-10-40, 50-15-35, 50-20-30, 50-25-25, 50-30-20,
55-0-45, 55-5-40, 55-10-35, 55-15-30, 55-20-25, 55-25-20, 55-30-15,
60-0-40, 60-5-35, 60-10-30, 60-15-25, 60-20-20, 60-25-15, 60-30-10,
10-35-55, 15-35-50, 20-35-45, 25-35-40, 30-35-35, 35-35-30, 40-35-25, 45-35-20, 50-35-15, 55-35-10, 60-35-5,
5-40-55, 10-40-50, 15-40-45, 20-40-40, 25-40-35, 30-40-30, 35-40-25, 40-40-20, 45-40-15, 50-40-10, 55-40-5, 60-40-0,
5-45-50, 10-45-45, 15-45-40, 20-45-35, 25-45-30, 30-45-25, 35-45-20, 40-45-15, 45-45-10, 50-45-5, 55-45-0,
5-50-45, 10-50-40, 15-50-35, 20-50-30, 25-50-25, 30-50-20, 35-50-15, 40-50-10, 45-50-5, 50-50-0,
5-55-40, 10-55-35, 15-55-30, 20-55-25, 25-55-20, 30-55-15, 35-55-10, 40-55-5, 45-55-0, 5-60-35, 10-60-30, 15-60-25, 20-60-20, 25-60-15, 30-60-10, 35-60-5, or 40-60-0% (w/w), apart from minor amounts of e.g. one or more further agent (usually not more than around 1.0% or less than 1.0%, such as around 0.5% or less, i.e. in the ppm ranges of around 0-1.000 ppm, or 0-5.000, or (per weight).

Thus, in some embodiment of the fluid composition, the lignin component is, or is around 5, 10, 15, 20, 25, 30, 35, 40, 45, 50, 55, or 60% (w/w).

In some embodiments, the liquid organic fraction is, or is around 0, 5, 10, 15, 20, 25, 30, 35, 40, 45, 50, 55, or 60% (w/w).

In some embodiments, the water content of the fluid composition is, or is around 5, 10, 15, 20, 25, 30, 35, 40, 45, 50, 55, or 60% (w/w). The water content can also be lower, present only in minor amounts—e.g. present in the lignin component and/or liquid organic fraction, or be absent.

In some embodiments, the fluid composition according to the invention may comprise a further agent, such as or around 0.05, 0.10, 0.15, 0.20, 0.25, 0.30, 0.35, 0.40, 0.45, 0.50 or more, such as or around 0.55, 0.60, 0.65, 0.70, 0.75, 0.8, 0.85, 0.90, 0.95, or 1.0% (w/w). Usually, the further agent is not present in an amount of more than 1% (w/w), preferably not more than around 0.5% (w/w). It has surprisingly been found that addition of further agents, such as hydrotropes can reduce the viscosity of fluid compositions containing lignin components e.g. from biomass refining plants, allowing higher amounts of lignin to be added to the fluid composition.

In the context of the present invention, the terms “about” and “around” can be used interchangeably, and mean variations generally accepted in the field, e.g. comprising analytical errors and the like. Commonly, “around” means variations of +/−1, 2, or 2.5% (w/w) based on the total composition, or on a specific compound, such as when relating to e.g. one or more further agents. In this case, as this further agent is present in lower amounts, “around” can also mean+/−0.1, 0.2, or 0.25% (w/w) based on the total composition or the respective agent.

Thus in summary, the first aspect of the invention concerns a fluid composition comprising a lignin component, an organic fraction in liquid state at 25° C., and optionally water and/or a further agent.

In particular, surprisingly and unexpectedly, the inventors have realized that the source of the lignin component has an influence on the quality of the fluid composition. In particular, a less polar lignin appears more suitable, such as a fluid composition, wherein said lignin component is not lignin from paper and pulp production, such as Kraft lignin, wherein said Kraft lignin being provided from biomass by a process known in the art as Kraft process/method (see e.g. Biermann, Christopher J. (1993) “Essentials of Pulping and Papermaking” San Diego: Academic Press, Inc.).

Without wanting to be bound by any theory, it is believed that an alkaline treatment has a negative effect on the lignin quality for uses related to the present invention, thus in some embodiments, said lignin component has not been provided by a Kraft method or another method comprising an alkaline treatment, such as by addition of NaOH or another base to provide a pH of around 10 or higher, at or around pH 11 or higher, or at or around pH 12 or higher.

Furthermore, it is believed that further modifications of the lignin or lignin component are not necessary to obtain a fluid composition according to the present invention, thus some embodiments concern desired a lignin component has not been esterified and/or subjected to an esterification step, e.g. as disclosed in WO2015/094098. It is believed to be an advantage that no further steps are needed, such as said modification of the lignin.

It appears a complex, if not to say an impossible task to measure polarity of a complex composition such as lignin. However, the inventors have developed a method to assess polarity, based on lignin's ion exchange capacity (LIEC; see e.g. Experimental section for further details). It became apparent that Kraft lignin has a significantly higher LIEC as e.g. 2G lignin that has not been subjected to an alkaline treatment. It is further believed that any wood, e.g. poplar wood and/or any other wood that e.g. is suitable for the paper industry, if processed without alkaline treatment, such as in a 2G process aiming at bioethanol production, will result in a lignin that is less polar, and thus suitable for providing a fluid composition according to the present invention.

In some embodiments, a fluid composition is provided comprising two or more fractions, wherein (a) the first fraction is an organic fraction in liquid state comprising one or more organic compounds such as one or more fat, and/or one or more oil; and (b) the second fraction comprises a lignin component having an Lignin Ion Exchange Capacity (LIEC) of 0.4 mol/kg dry matter or less. In further embodiments, said lignin component has a Lignin Ion Exchange Capacity (LIEC) of 0.3 mol/kg dry matter (DM) or less, such as 0.25 mol/kg DM or less, such as 0.20 mol/kg DM or less, such as 0.15 mol/kg DM or less, or such as 0.10 mol/kg DM or less. In some embodiments, the LIEC can be in the range of 0.05-0.30, 0.10-0.25, or 0.10-0.15 mol/kg DM. In further embodiments, the lignin component is significantly less polar than Kraft lignin, such as assessed by LIEC measurement, such as having a LIEC at least 0.10, 0.11, 0.12, 0.13, 0.14, 0.15, 0.16, or 0.17 mol/kg DM lower than the LIEC of Kraft lignin.

Without wanting to be bound by any theory, it is believed that it is an advantage that the lignin component according to the invention is less hygroscopic than e.g. Kraft lignin. Thus according to some embodiments, a fluid composition is provided, wherein said lignin component is significantly less hygroscopic, such as binding at least 20, 25, 30, 35, 40, 45, 50, 55, 60, 65, 70, 75, 80, 85, 90, 95 or 100% (w/w) less water when compared to Kraft lignin.

Without wanting to be bound by any theory, it is believed that it is an advantage that the lignin component according to the invention swells less than e.g. Kraft lignin. Thus according to some embodiments, a fluid composition is provided, wherein said lignin component is swelling significantly less than Kraft lignin, such as swelling at least 20, 25, 30, 35, 40, 45, 50, 55, 60, 65, 70, 75, 80, 85, 90, 95 or 100% less, and optionally wherein said swelling is determined as change in particle size upon suspension in water or another suitable medium after 60 min.

Further desired features of the fluid composition according to the present invention concern its improved stability and or pumpability when compared to similar compositions. Thus, according to some embodiments, the fluid composition is significantly more stable and/or pumpable when compared to a similar composition prepared with Kraft lignin.

In the context of the present invention, the term “pumpability” is meant to comprise that the fluid composition has a viscosity of 1 Pa·s or less, such as 0.9 Pa·s or less, such as 0.8 Pa·s or less, such as 0.7 Pa·s or less, such as 0.6 Pa·s or less, such as 0.5 Pa·s, such as 0.4 Pa·s or less, such as 0.3 Pa·s or less, such as 0.2 Pa·s or less, or such as 0.1·Pa·s or less at a shear rate of 100 s⁻¹. According to a preferred embodiment, the viscosity is 0.5 Pa·s, or less, or event around 0.25, again measured at a shear rate of 100 s⁻¹, wherein said viscosity is measured as overage over at time period 10 min. In some embodiments, said time period is 5 or 15 min. It has been observed that viscosity is not constant, and tends to increase with time.

Further desired features of the fluid composition according to the present invention concern its improved stability and or pumpability when compared to similar compositions. Thus, according to some embodiments, the fluid composition possesses a significant short term, medium term, or long term stability and/or pumpability, wherein said short, medium, and long term are periods of time in the range of 1-60 min, >1-24 h, or >24 h, respectively. In particular, in some embodiments a fluid composition is provided with an increased short term, medium term and/or long-term stability and/or pumpability, when compared to a similar composition prepared with Kraft lignin. In further embodiments, said short term time period can be 1, 2, 5, 10, 15, 20, 30, 45, or 60 min. In other embodiments, said medium term time period can be 90 min, 2 h, 4 h, 6 h, 8 h, 10 h, 12 h, 18 h, 24 h. In embodiments relating to long term stability, said long term time period can 25 h, 30 h, 40 h, 2 d, 3 d, 4 d, 5 d, 6 d, 1 week, 2 weeks, 3 weeks, 1 month, 2, months, 3 month, 4 months, 5 months, 6 months, or more than 6 month.

Concerning a definition for stability, in the context of the present invention the term “stability” are meant to comprise that no more than 5.0, 4.0, 3.0, 2.0, 1.0 or 0.5% (w/w) of any one of the fractions (e.g. water, liquid organic fraction, and/or lignin component) of said fluid composition will separate after said specified period of time. It is understood, that in some embodiments a fluid composition is provided, wherein occasional or constant gentle stirring, agitation, and/or re-circulation but no high shear mixing may be required for maintaining said stability and/or pumpability. “No high shear mixing” can e.g. be defined as requiring significantly less—e.g. at least 5×, 10×, 25×, 50× or 100× less—mixing/shearing energy compared to the mixing provided by an IKA Ultra Turrax T25 high-shear mixer at 3.000 rpm and a volume of around 200 ml for 1 min. “No high shear mixing” can also be defined as mixing with a non-high shear force providing mixer.

According to the first embodiment, the preset invention concerns a fluid composition according to any one of the preceding claims comprising two or more fractions, wherein (a) the first fraction is an organic fraction in liquid state at room temperature, said organic fraction comprising one or more organic compounds such as one or more fat, and/or one or more oil; and (b) the second fraction comprises one or more lignin component.

In some embodiments, the fluid composition comprises 5-60% (w/w) lignin component, 0-40% (w/w) organic fraction, 0-60% (w/w) water, and 0-1.0 or 0-0.5% (w/w) further agent. According to some embodiments, the fluid composition the water is comprised (i) in the first fraction, such as an oil- and/or fat-water-emulsion, as a homogenous solution of oil and/or fat and water; (ii) as a third, aqueous fraction; or (iii) as a combination of (i) and (ii).

In some embodiments, the Lignin Ion Exchange Capacity is around 0.40, 0.35, 0.30, 0.25, 0.20, 0.15, 0.10, mol/kg dry matter or less; in the range of 0.10-0.20, 0.20-0.30, 0.30-0.40 mol/kg dry matter; and/or in the range of 0.05-0.40, 0.10-0.30, or 0.10-0.20 mol/kg DM.

As indicated earlier, the fluid composition according to the invention may comprise one or more further agent, such as an agent is selected from the group comprising or consisting of one or more dispersing agent(s), surfactant(s), hydrotropic agent(s), emulsifier(s), preserving agent(s), and any combination thereof. According to one embodiment, said one or more further agent is present in the range of 0.001% to 5% (w/w).

According to invention, the various constituents of the fluid composition are intermixed. Thus, according to some embodiments, the one or more fat, oil, lignin component, water, further agent, dispersing agent, surfactant, hydrotropic agent, emulsifier, preserving agent, and any combination thereof are in a state of being intermixed. In some embodiments, said state of being intermixed is selected from the group comprising or consisting of being intermixed as a solution; being intermixed as a suspension; being intermixed as an emulsion; being intermixed as a dispersion; being intermixed as a slurry; and any combination thereof.

The lignin component comprised in the fluid composition may comprise e.g. cellulose and/or hemicellulose and/or ash. Thus, in some embodiments, said lignin component comprises cellulose in an amount of 2,000-300,000 ppm, such as 3,000-180,000 ppm, e.g. 4,000-160,000 ppm, for example 5,000-140,000 ppm, such as 6,000-120,000 ppm, 7,000-100,000 ppm, for example 8,000-80,000 ppm, such as 9,000-70,000 ppm, e.g. 10,000-60,000 ppm, 12,000-50,000 ppm, such as 14,000-50,000 ppm, e.g. 16,000-40,000 ppm, 18,000-30,000 ppm, such as 20,000-28,000 ppm, for example 22,000-26,000 ppm (w/w) in relation to said fluid composition. In some embodiments, said lignin component comprises hemicellulose in an amount of 2,000-200,000 ppm, such as 3,000-180,000 ppm, e.g. 4,000-160,000 ppm, for example 5,000-140,000 ppm, such as 6,000-120,000 ppm, 7,000-100,000 ppm, for example 8,000-80,000 ppm, such as 9,000-70,000 ppm, e.g. 10,000-60,000 ppm, 12,000-50,000 ppm, such as 14,000-50,000 ppm, e.g. 16,000-40,000 ppm, 18,000-30,000 ppm, such as 20,000-28,000 ppm, for example 22,000-26,000 ppm (w/w) in relation to said fluid composition. In some embodiments, said lignin component comprises ash in an amount of 2,000-200,000 ppm, such as 3,000-180,000 ppm, e.g. 4,000-160,000 ppm, for example 5,000-140,000 ppm, such as 6,000-120,000 ppm, 7,000-100,000 ppm, for example 8,000-80,000 ppm, such as 9,000-70,000 ppm, e.g. 10,000-60,000 ppm, 12,000-50,000 ppm, such as 14,000-50,000 ppm, e.g. 16,000-40,000 ppm, 18,000-30,000 ppm, such as 20,000-28,000 ppm, for example 22,000-26,000 ppm (w/w) in relation to said fluid composition.

Dispersing agents are known in the art, and according to some embodiments, the fluid composition comprises one or more dispersing agent is selected from the group comprising or consisting of non-ionic, anionic, cationic and amphoteric dispersing agent(s) and any combination and/or compatible mixture thereof. Such agents can be present in different concentrations. In some embodiments, said one or more dispersing agent is present in said fluid composition in an amount of 10-50,000 ppm or 200-20,000 ppm, such as 300-18,000 ppm, e.g. 400-16,000 ppm, for example 500-14,000 ppm, such as 600-12,000 ppm, 700-10,000 ppm, for example 800-8,000 ppm, such as 900-7,000 ppm, e.g. 1,000-6,000 ppm, 1,200-5,000 ppm, such as 1,400-5,000 ppm, e.g. 1,600-4,000 ppm, 1,800-3,000 ppm, such as 2,000-2,800 ppm, for example 2,200-2,600 ppm (w/w) in relation to said fluid composition. Suitable dispersing agents may be selected from the group comprising: Lutensol AP10, AP8, AP7 and AP6 from BASF. The Lutensol AP series consists of ethoxylated nonylphenols, C9H19-C6C4O(CH2CH2O)xH, where x is the numeric portion of the product name. Another suitable dispersing agent may be Tergiotol NP-9 from Union Carbide, with essentially the same composition as the Lutensol series.

The above and below referred modes of being intermixed and inclusion of water and dispersing agent have proven advantageous in the goal of obtaining stable fluid compositions according to the first aspect of the present invention. It should be noted however, that it is possible to obtain stable fluid composition according to the first aspect of the present invention without inclusion or separately adding of any further agents, such as dispersing agents or the like. This is quite surprising in view of the prior art teaching.

Surfactants are known in the art, and according to some embodiments, the fluid composition comprises one or more surfactant selected from the group comprising or consisting of anionic, cationic, zwitterionic and nonionic surfactants, and any combination and/or compatible mixture thereof. In some embodiments, said one or more surfactant is present in said fluid composition in an amount of 10-50,000 ppm or 200-20,000 ppm, such as 300-18,000 ppm, e.g. 400-16,000 ppm, for example 500-14,000 ppm, such as 600-12,000 ppm, 700-10,000 ppm, for example 800-8,000 ppm, such as 900-7,000 ppm, e.g. 1,000-6,000 ppm, 1,200-5,000 ppm, such as 1,400-5,000 ppm, e.g. 1,600-4,000 ppm, 1,800-3,000 ppm, such as 2,000-2,800 ppm, for example 2,200-2,600 ppm (w/w) in relation to said fluid composition.

Hydrotropes are known in the art, and according to some embodiments, the fluid composition comprises one or more hydrotrope is selected from the group comprising or consisting of: non-ionic, anionic, cationic and amphoteric hydrotropes and any combination and/or compatible mixtures thereof. In some embodiments, said one or more hydrotrope is present in said fluid composition in an amount of 10-50,000 ppm or 200-40,000 ppm, such as 300-30,000 ppm, e.g. 400-20,000 ppm, for example 500-15,000 ppm, such as 600-12,000 ppm, 700-10,000 ppm, for example 800-8,000 ppm, such as 900-7,000 ppm, e.g. 1,000-6,000 ppm, 1,200-5,000 ppm, such as 1,400-5,000 ppm, e.g. 1,600-4,000 ppm, 1,800-3,000 ppm, such as 2,000-2,800 ppm, for example 2,200-2,600 ppm (w/w) in relation to said fluid composition. Thus in some embodiments of the first aspect of the present invention said fluid composition furthermore comprises a hydrotropic agent. In such embodiments, said hydrotropic agent may be selected from the group comprising: non-ionic, anionic, cationic and amphoteric hydrotropic agents and compatible mixtures thereof. Suitable hydrotropes may be selected from the group comprising benzoic acid, alkyl-benzoic acid, Butyldiglycol, butanol, propanol, lignosulphonates, toluenesulphonates, xylenesulphonates and cumesulphonates, caprionates, caprylates, glucose and sodium benzoate. Suitable hydrotropes may also be selected from the Sokalan CP series from BASF (e.g. Eg. Sokalan CP9-Maleic acid-olefin copolymer, sodium salt; CP10-Modified polyacrylic acid, sodium salt, CP10S-Modified polyacrylic acid, sodium salt) comprising polyacrylates and maleic acid-acrylic acid co-polymers and the like.

The above referred modes of being intermixed and inclusion of water and further agent(s), such as one or more hydrotopic agent have proven advantageous in the goal of stabilizing and reducing the viscosity of compositions according to the first aspect of the present invention.

Emulsifiers are known in the art, and according to some embodiments, the fluid composition comprises one or more emulsifier is selected from the group comprising or consisting of sodium phosphate(s), sodium stearoyl lactylate cationic, lecithin, DATEM (diacetyl tartaric acid ester of monoglyceride), and any combination and/or compatible mixture thereof. In some embodiments, said one or more emulsifier is present in said fluid composition in an amount of 10-50,000 ppm or 200-20,000 ppm, such as 300-18,000 ppm, e.g. 400-16,000 ppm, for example 500-14,000 ppm, such as 600-12,000 ppm, 700-10,000 ppm, for example 800-8,000 ppm, such as 900-7,000 ppm, e.g. 1,000-6,000 ppm, 1,200-5,000 ppm, such as 1,400-5,000 ppm, e.g. 1,600-4,000 ppm, 1,800-3,000 ppm, such as 2,000-2,800 ppm, for example 2,200-2,600 ppm (w/w) in relation to said fluid composition.

In some embodiments of the first aspect of the present invention, said fluid composition furthermore comprises a preservative. Preserving agents are known in the art, and according to some embodiments, the fluid composition comprises one or more preserving agent selected from the group comprising or consisting of one or more carboxylate, benzoate, benzoic acid derivative such as parabene(s), aldehyde(s), thiazine(s), organic acid(s), salt(s) of organic acid(s) and the like, and any combination thereof. In some embodiments, said one or more preserving agent is present in said fluid composition in an amount of 10-50,000 ppm or 20-10,000 ppm, such as 30-8,000 ppm, e.g. 40-6,000 ppm, for example 50-5,000 ppm, such as 60-4,000 ppm, 70-3,000 ppm, for example 80-2,000 ppm, such as 90-1,500 ppm, e.g. 100-1,200 ppm, 120-1,000 ppm, such as 140-800 ppm, e.g. 160-600 ppm, 180-400 ppm, such as 200-300 ppm, for example 2,200-250 ppm (w/w) in relation to said fluid composition. The above referred modes of being intermixed and inclusion of water and preservative have proven advantageous in the goal of stabilizing and reducing microbial growth according to the first aspect of the present invention.

As mentioned earlier, the fluid composition according to the invention comprises lignin and/or a lignin component. This lignin and/or lignin component can e.g. be characterized by its dry matter (DM) content. In some embodiments, the dry matter content of said lignin component in said fluid composition is 1.0-99% (w/w), 10-99% (w/w) or 20-95% (w/w), such as 21-94% (w/w), e.g. 22-93% (w/w), such as 23-92% (w/w), such as 24-91% (w/w), for example 25-90% (w/w), such as 26-89% (w/w), such as 27-88% (w/w), for example 28-87% (w/w), e.g. 29-86% (w/w), such as 30-85% (w/w), such as 31-84% (w/w), such as 32-83% (w/w), such as 33-82% (w/w), for example 34-81% (w/w), such as 35-80% (w/w), e.g. 36-79% (w/w), such as 37-78% (w/w), e.g. 38-77% (w/w), e.g. 39-76% (w/w), such as 40-75% (w/w), such as 41-74% (w/w), such as 42-73% (w/w), such as 43-72% (w/w), for example 44-71% (w/w), such as 45-70% (w/w), e.g. 46-69% (w/w), such as 47-68% (w/w), e.g. 48-67% (w/w), e.g. 49-66% (w/w), such as 50-65% (w/w), such as 51-64% (w/w), such as 52-63% (w/w), such as 53-62% (w/w), for example 54-61% (w/w), such as 55-60% (w/w), e.g. 56-59% (w/w), such as 57-58% (w/w). These ranges have proven advantageous in reaching satisfactorily fluid compositions according to the first aspect of the present invention.

The lignin and/or lignin component may also comprise sulfur. In some embodiments, the fluid composition comprises a lignin component, wherein the sulfur content—based on the dry matter content of said lignin component—is 2.0% (w/w) or less, such as 1.4% (w/w) or less, such as 1.3% (w/w) or less, for example 1.2% (w/w) or less, such as 1.1% (w/w) or less, e.g. 1.0% (w/w) or less, such as 0.9% (w/w) or less, for example 0.8% (w/w) or less, such as 0.7% (w/w) or less, e.g. 0.6% (w/w) or less, e.g. 0.5% (w/w) or less, such as 0.4% (w/w) or less, for example 0.3% (w/w) or less, such as 0.2% (w/w) or less, or 0.1% (w/w) or less, such as 0.09% (w/w) or less, such as 0.08% (w/w) or less, e.g. 0.07% (w/w) or less, e.g. 0.06% (w/w) or less, such as 0.05% (w/w) or less, for example 0.04% (w/w) or less, such as 0.03% (w/w) or less, e.g. 0.02% (w/w) or less, such as 0.01% (w/w) or less. Generally, a low sulfur content seems preferred in view of e.g. environmental and/or economical concerns, in particular when the fluid composition is used as fuel. Thus the above stated low sulfur contents of the lignin component of the fluid composition according to the first aspect of the present invention contribute in making the fluid composition suitable for use as an environmentally friendly fuel.

Concerning the grain and or particle size of the lignin component in the fluid composition of the current invention, this may be of different sizes and/or size distributions. In some embodiments, the lignin component in said fluid composition is having an average grain size of 1-2000 μm, 1-1500 μm, 1-1200 μm, 1-1000 μm, 1-800 μm, 1-600 μm, 1-500 μm, 1-450 μm, such as 1.5-430 μm, e.g. 2-420 μm, such as 3-410 μm, for example 4-400 μm, e.g. 5-390 μm, such as 6-380 μm, e.g. 7-370 μm, such a 8-360 μm, 9-350 μm, for example 10-340 μm, e.g. 12-330 μm, such as 14-320 μm, such as 16-310 μm, for example 18-300 μm, e.g. 20-290 μm, such as 22-280 μm, e.g. 25-270 μm, such a 30-260 μm, 35-250 μm, for example 40-240 μm, e.g. 45-230 μm, such as 50-220 μm, for example 60-210 μm, for example 70-200 μm, e.g. 80-190, for example 90-180 μm, e.g. 100-170 μm, such a 110-160 μm, 120-150 μm, for example 130-140 μm. Obviously, the particle size may vary, depending of the time of measurement. It is generally believed, that the particle size may increase upon providing the fluid composition by intermixing the lignin component. An increase in average grain/particle size distribution may be caused by swelling, and without wanting to be bound by any theory, this is believed to also be correlated to the lignin/lignin component's hygroscopy. There are different methods known in the art for determining said particle or grain size. According to some embodiments, said average grain or particle size being measured before or after providing said fluid composition. In some further embodiments, said grain or particle size being measured by laser diffraction spectroscopy, or e.g. by a Malvern Mastersizer. In some embodiments, dry lignin particle size is measured with a sieve tower (for a standard method, refer to ASTM C136/C136M-14) or a Retch Camsizer. In some embodiments, wet samples, such as fluid composition according to the present invention (i.e. were the particles are intermixed with water and or the liquid organic fraction) particle size is determined using laser diffraction. It is believed that particle size can also be determined in dry or wet samples using microscopy, or other common methods know in the art. The above stated average grain sizes have proven advantageous in the goal of obtaining stable fluid compositions according to the first aspect of the present invention. One mode of measuring the average grain size of the lignin component is by dynamic light scattering. In one embodiment of the first aspect of the present invention the lignin component originates from a lignocellulosic biomass having been subjected to a hydrothermal pretreatment followed by a hydrolysis of at least part of the cellulose and at least part of the hemicellulose present in said lignocellulosic biomass.

As stated herein, the quality and/or physical properties of the lignin and/or lignin component appear important for the invention, and the inventors have discovered that the quality of the fluid composition according to the current invention are improved when not using Kraft lignin or lignin that had been subjected to an alkaline treatment. It is believed that the process used for providing the lignin or lignin component is more important than the biological source of it. Thus, according to some embodiments, the lignin component originates from a lignocellulosic biomass having been subjected to a hydrothermal pretreatment followed by a hydrolysis of at least part of the cellulose and at least part of the hemicellulose present in said lignocellulosic biomass. According to some embodiment, said lignin component originates from a lignocellulosic biomass having been subject to a hydrothermal pretreatment followed by a hydrolysis of at least part of the cellulose and at least part of the hemicellulose present in said lignocellulosic biomass; and optionally followed by a fermentation, such as an alcohol fermentation. Different hydrolysis methods appear suitable. Thus, according to some embodiments, said hydrolysis is an acid catalyzed hydrolysis, an enzymatic hydrolysis or a combination of acid/enzyme-catalyzed hydrolysis. Schemes for processing lignocellulosic biomass, including specific process steps as well as overall schemes for converting a lignocellulosic biomass to soluble saccharides and a fibrous fraction comprising a lignin component, are the subject of numerous published patents and patent applications. See e.g. WO 94/03646; WO 94/29474; WO 2006/007691; US2007/0031918; WO 2008/112291; WO 2008/137639; EP 2 006 354; US 2009/0326286; US 2009/0325251; WO 2009/059149; US 2009/0053770; EP 2 169 074; WO 2009/102256; US 2010/0065128; US 2010/0041119; WO 2010/060050; WO2007009463 A2, WO2007009463 A1; WO2011125056 A1; and WO2009125292 A2, each of which is hereby incorporated by reference in entirety.

The above embodiments of the first aspect of the present invention and relating to the biorefining process of a lignocellulosic biomass as a source to obtain the lignin component of the fluid composition of the first aspect of the present invention have proven especially advantageous as this process will provide a lignin component having the desired characteristics for obtaining stable fluid compositions having a low sulfur content.

According to the invention, the lignin component may also be characterized by its average molecular weight. According to some embodiments, the a fluid composition is provide, wherein said lignin component is having an average molecular weight (Da) of 1,000 or above, 1,500 or above, 2,000 or above, 2,500 or above, 3,000 or above, such as 3,500 or above, e.g. 4,000 or above, such as 5,000 or above, for example 5,500 or above, such as 6,000 or above, e.g. 7,000 or above, for example 8,000 or above, such as 9,000 or above, for example 10,000 or above, such as 12,000 or above, e.g. 14,000 or above, for example 16,000 or above, e.g. 18,000 or above, e.g. 20,000 or above, such as 25,000 or above, e.g. 30,000 or above, such as 35,000 or above, for example 40,000 or above, such as 45,000 or above, e.g. 50,000 or above, such as 55,000 or above, e.g. 60,000 or above, such as 65,000 or above, e.g. 70,000 or above, such as 75,000 or above, for example 80,000 or above, such as 85,000 or above, e.g. 90,000 or above, such as 95,000 or above, or 100,000 or above.

As mentioned earlier, a variety of different lignin sources appear suitable in the context of the current invention. According to some embodiments, a fluid composition is provided, wherein said lignin component originates from a lignocellulosic biomass obtained from an annual or a perennial plant. Thus according to some embodiments, the lignin component may originate from a lignocellulosic biomass obtained, obtainable or derived from the group comprising or consisting of one or more of: cereal, wheat, wheat straw, rice, rice straw, corn, corn fiber, corn cobs, corn stover, hardwood bulk, softwood bulk, sugar cane, sweat sorghum, bagasse, nut shells, empty fruit bunches, grass, cotton seed hairs, barley, rye, oats, sorghum, brewer's spent grains, palm waste material, wood, soft lignocellulosic biomass, and any combination thereof.

It is apparent that in view of different sources of biomass, including different methods of biomass processing, some impurities may be present. According to some embodiments, a fluid composition is provided, wherein said lignin component comprises one or more impurities originating from its mode of production, such as enzyme residues, yeast residues, foam depressant(s), clean in place (CIP) compounds, salts and the like. According to some embodiments, said lignin component comprises impurity/impurities originating from compounds native for the lignocellulosic material, such as cellulose residues, hemicellulose residues, monomeric sugar compounds, dimeric sugar compounds, oligomeric sugar compounds, carbohydrate residues, wax residues, minerals, ash, silica (SiO₂), silica-comprising compounds and/or compositions, salts, organic acids, and the like, and any combination thereof. According to some embodiments, the purity of said lignin component is 50% (w/w) or more, such as 52% (w/w) or more, for example 54% (w/w) or more, such as 56% (w/w) or more, e.g. 58% (w/w) or more, such as 60% (w/w) or more, such as 62% (w/w) or more, for example 64% (w/w) or more, such as 66% (w/w) or more, e.g. 68% (w/w) or more, such as 70% (w/w) or more, such as 72% (w/w) or more, for example 74% (w/w) or more, such as 76% (w/w) or more, e.g. 78% (w/w) or more, such as 80% (w/w) or more, such as 82% (w/w) or more, for example 84% (w/w) or more, such as 86% (w/w) or more, e.g. 88% (w/w) or more, such as 90% (w/w) or more, such as 92% (w/w) or more, for example 94% (w/w) or more, such as 96% (w/w) or more, e.g. 98% (w/w) or more. According to an embodiment, the purity of said lignin or lignin component is determined based on content of Klason lignin. According to an embodiment, the purity of said lignin or lignin component is determined based on content of acid insoluble lignin. According to some embodiments, the corresponding percentage constituting impurities may be any one or more impurity as defined earlier in this paragraph.

Apart from lignin or one or more lignin component, and optionally water and/or one or more further agents, the fluid composition according to the present invention comprises an organic fraction. The organic fraction may in turn consist or comprise one or more organic compounds. This organic substance which imparts liquid character to the fluid composition is believed to act as a carrier and/or matrix for the solid fraction comprising the lignin component. In some embodiments, the content of said organic fraction in said fluid composition is at least 2, 5, 10, 15, 20, 25, 30, 35, 40, 45, 50, 55, 60, 65, 70, 75, 80, 85, 90, or 95% (w/w) or more, such as 2-95% (w/w), such as 4-78% (w/w), e.g. 6-76% (w/w), such as 8-74% (w/w), e.g. 10-72% (w/w), such as 12-70% (w/w), e.g. 14-68% (w/w), such as 16-66% (w/w), for example 18-64% (w/w), such as 20-62% (w/w), e.g. 22-60% (w/w), for example 24-58% (w/w), such as 26-56% (w/w), such as 28-54% (w/w), such as 30-52% (w/w), 32-50% (w/w), e.g. 34-48% (w/w), such as 36-46% (w/w), such as 38-44% (w/w), for example 40-42% (w/w). In some embodiments, said organic fraction comprises or consists essentially of an organic solvent, a distillate and/or a residue from a hydrocarbon distillation. In some embodiments, said distillate is selected from the group comprising or consisting of one or more mineral oil, kerosene, diesel, No. 2 fuel oil, No. 3 fuel oil, No. 4 fuel oil fuel oil, No. 5 fuel oil, No. 6 fuel oil, and No. 7 fuel oil, and any mixture(s) thereof. In other embodiments, the fluid composition may comprise one or more organic compound of plant origin or animal origin. In some embodiments, the one or more organic compound of said liquid organic fraction is an oil of plant origin or a fat of animal origin. In some embodiments, said one or more organic compound of said liquid organic fraction is an oil originating from pyrolysis of a biomass, such as a cellulosic or lignocellulosic material or wherein said oil is a pyrolysis oil originating from pyrolysis of a lignin component. In some embodiments, the one or more organic compound of said liquid organic fraction is an oil originating from pyrolysis of a polymer, such as a synthetic plastic or synthetic elastomer. In some embodiments, the one or more organic compound of said liquid organic fraction is selected from the group comprising or consisting of glycerol, biodiesel, synfuel, biomass to liquid (BTL) diesel, gas to liquid (GTL) diesel, coal to liquid (CTL) diesel, and any combination thereof. In some embodiments, the one or more organic compound of said liquid organic fraction originates from treatment of a biomass with water and/or other polar liquid(s), such as ethanol or methanol, which may include treatment under supercritical conditions. In an embodiment of the first aspect of the present invention the biomass which has been treated with water or other polar liquid(s), optionally under supercritical conditions, said biomass may be selected from the group comprising a lignocellulosic material, cellulose and a lignin component.

The above defined characteristics of the organic substance comprised in the liquid fraction of the fluid composition according to the first aspect of the present invention are very well suited for this fluid composition, especially when used as a liquid fuel.

Concerning biomass treatment in relation to the current invention, in an embodiment, a fluid composition is provided, wherein the said biomass treatment comprises treatment under supercritical conditions. In further embodiments, said biomass which has been treated with water or other polar liquid(s) under supercritical conditions may be selected from the group comprising or consisting of one or more lignocellulosic material, cellulose, lignin component, and any combination thereof.

According to the invention, a fluid composition is provided, wherein said liquid organic fraction or compound of said liquid organic fraction is in itself a mixture of two or more such organic substances, such as three or more such organic substances, e.g. four or more such organic substances, such as five or six or more of such organic substances.

In mixing different individual organic substances in order to obtain the organic substance to be comprised in the liquid fraction of the fluid composition according to the first aspect of the present invention it may be possible to impart specific and beneficial physical characteristics to the liquid fraction of the fluid composition not obtainable by using a single component fluid fraction.

In some embodiments, the sulfur content of said liquid organic fraction, and/or the one or more organic compound and/or substance of said organic liquid fraction is 5.0% (w/w) or less, such as 4.5% (w/w) or less, for example 4.0% (w/w) or less, such as 3.8% (w/w) or less, e.g. 3.6% (w/w) or less, for example 3.4% (w/w) or less, e.g. 3.2% (w/w) or less, such as 3.0% (w/w) or less, for example 2.8% (w/w) or less, e.g. 2.6% (w/w) or less, for example 2.4% (w/w) or less, e.g. 2.2% (w/w) or less, such as 2.0% (w/w) or less, for example 1.8% (w/w) or less, such as 1.6% (w/w) or less, for example 1.4% (w/w) or less, e.g. 1.2% (w/w) or less, such as 1.0% (w/w) or less, for example 0.8% (w/w) or less, such as 0.4% (w/w) or less, such as 0.2% (w/w) or less, for example 0.1% (w/w) or less, such as 0.08% (w/w) or less, e.g. 0.06% (w/w) or less, such as 0.04% (w/w) or less, e.g. 0.02% (w/w) or less, for example 0.01% (w/w) or less, such as 0.008% (w/w) or less, e.g. 0.006% (w/w) or less, such as 0.004% (w/w) or less, e.g. 0.002% (w/w) or less, such as 0.001% (w/w), such as 800 ppm or less, e.g. 600 ppm or less, such as 400 ppm or less, e.g. 200 ppm or less, for example 100 ppm or less, such as 50 ppm (w/w) or less. The above stated low sulfur contents of the lignin component of the fluid composition according to the first aspect of the present invention makes the fluid composition suitable for use as an environmentally friendly fuel.

Seeing that the lignin component originating from biorefining a lignocellulosic biomass by subjecting it to a hydrothermal pretreatment followed by a hydrolysis will have a somewhat hydrophobic character depending on the residual amount of cellulose and hemicellulose, it will in certain formulations be advantageous to make sure that the organic substance of said liquid fraction of the fluid composition according to the first aspect of the present invention is an organic substance being immiscible with water thereby itself being hydrophobic. Thus in some embodiments, said liquid organic fraction, organic compound or substance of said liquid organic fraction is immiscible with water.

According to some embodiments, said organic fraction, one or more organic compound or substance at 25° C. is having a viscosity of 0.0005-10,000 CSt, such as 0.0010-9,000 CSt, e.g. 0.0050-8,000 CSt, for example 0.01-6,000 CSt, for example 0.05-4,000 CSt, such as 0.1-2,000 CSt, e.g. 0.5-1,000 CSt, such as 1.0-800 CSt, e.g. 5.0-600 CSt, such as 10-400 CSt, for example 50-300 CSt, such as 100-200 CSt. According to some embodiments, said fluid composition, organic fraction, one or more organic compound or substance, wherein said organic substance at 50° C. is having a viscosity of 0.0004-2,000 CSt, such as 0.0010-1,500 CSt, e.g. 0.0050-1,000 CSt, for example 0.01-800 CSt, for example 0.05-600 CSt, such as 0.1-400 CSt, e.g. 0.5-200 CSt, such as 1.0-100 CSt, e.g. 5.0-80 CSt, such as 10-70 CSt, for example 20-50 CSt, such as 30-40 CSt. According to some embodiments, said fluid composition, organic fraction, one or more organic compound or substance, wherein said organic substance at 75° C. is having a viscosity of 0.0002-200 CSt, such as 0.0001-150 CSt, e.g. 0.001-100 CSt, for example 0.005-80 CSt, such as 0.01-60 CSt, e.g. 0.05-40 CSt, such as 0.05-20 CSt, for example 0.1-10 CSt, such as 0.5-5 CSt, for example 1.0-3 CSt. The above stated viscosities of the organic substance of said liquid fraction of the fluid composition according to the first aspect of the present invention shall be understood to be independently chosen and shall be understood to define the ranges of viscosities which impart liquid character to said liquid fraction of the fluid composition according to the first aspect of the present invention.

According to the invention, the fluid composition may or may not comprise water. In some embodiments a fluid composition is provided, wherein the content of said water in said fluid composition is less than 90, 85, 80, 75, 70, 65, 60, 55, 50, 45, 40, 35, 30, 25, 20, 15, 12, 10, 8, 7, 6, 5, 4, 3, 2, 1, 0.5% (w/w) such as in the range of 2-80% (w/w), such as 4-78% (w/w), e.g. 6-76% (w/w), such as 8-74% (w/w), e.g. 10-72% (w/w), such as 12-70% (w/w), e.g. 14-68% (w/w), such as 16-66% (w/w), for example 18-64% (w/w), such as 20-62% (w/w), e.g. 22-60% (w/w), for example 24-58% (w/w), such as 26-56% (w/w), such as 28-54% (w/w), such as 30-52% (w/w), 32-50% (w/w), e.g. 34-48% (w/w), such as 36-46% (w/w), such as 38-44% (w/w), for example 40-42% (w/w).

In some embodiments a fluid composition is provided, wherein the ratio lignin component:water is selected from the range of 0.4-8.0, such as 0.5-7.9, e.g. 0.6-7.8, such as 0.7-7.6, for example 0.8-7.5, for example 0.9-7.4, such as 1.0-7.3, for example 1.1-7.2, e.g. 1.2-7.1, such as 1.3-7.0, for example 1.4-6.9, such as 1.5-6.8, such as 1.6-6.7, such as 1.7-6.6, for example 1.8-6.5, for example 1.9-6.4, such as 2.0-6.3, for example 2.1-6.2, e.g. 2.2-6.1, such as 2.3-6.0, for example 2.4-5.9, such as 2.5-5.8, such as 2.6-5.7, such as for example 2.8-5.5, for example 2.9-5.4, such as 3.0-5.3, for example 3.1-5.2, e.g. 3.2-5.1, such as 3.3-5.0, for example 3.4-4.9, such as 3.5-4.8, such as 3.6-4.7, such as 3.7-4.6, for example 3.8-4.5, for example 3.9-4.4, such as 4.0-4.3, for example 4.1-4.2, all ratios being based on dry matter content of said lignin component.

The viscosity of the fluid composition according to invention may be e.g. be as follows. In some embodiments said fluid composition at 25, 50 or 75° C. is having a viscosity of 20-10,000 CSt, such as 50-8,000 CSt, for example 100-6,000 CSt, such as 200-4,000 CSt, such as 400-2,000 CSt, e.g. 500-1,000 CSt, such as 600-800 CSt. In some embodiments said said fluid composition at 25, 50 or 75° C. is having a viscosity of 5-2,000 CSt, such as 10-1,000 CSt, for example 20-800 CSt, such as 50-600 CSt, e.g. 100-400 CSt, such as 200-300 CSt. In some embodiments said fluid composition at 25, 50 or 75° C. is having a viscosity of 2-200 CSt, such as 5-150 CSt, e.g. 10-120 CSt, such as 20-100 CSt, for example 30-80 CSt, such as 40-60 CSt. The above-specified viscosities of the fluid composition are independently examples of what is understood to be a “fluid composition” in the present application and in the appended claims, such as defined according to the first aspect. These specified viscosities of the fluid composition according to the first aspect of the present invention have proven advantageous, because such viscosities will ensure that said fluid composition is pumpable. This is especially important in case the fluid composition is going to be used as a fuel.

According to some embodiments, a fluid composition is provided having a lower heating value of 4-37 MJ/kg, such as 5-36 MJ/kg, for example 6-35 MJ/kg, such as 7-34 MJ/kg, for example 8-33 MJ/kg, e.g. 9-32 MJ/kg, such as 10-31 MJ/kg, for example 11-30 MJ/kg, such as 12-29 MJ/kg, e.g. 13-28 MJ/kg, such as 14-27 MJ/kg, such as 15-26 MJ/kg, for example 16-25 MJ/kg, such as 17-24 MJ/kg, for example 18-23 MJ/kg, e.g. 19-22 MJ/kg, such as 20-21 MJ/kg. These lower heating values of the fluid composition make the fluid composition useful as a fuel.

According to some embodiments, a fluid composition is provided that is stable and/or pumpable for 2 weeks or more, such as 3 weeks or more, e.g. 4 weeks or more, such as 6 weeks or more, for example 7 weeks or more, such as 8 weeks or more, such as 2 months or more, e.g. 3 months or more, for example 4 months or more, such as 5 months or more, or 6 months or more; in the sense that no more than 5.0, 4.5, 4.0, 3.5, 3.0, 2.5, 2.0, 1.5, 1.0 or 0.5% (w/w) of any one of the fractions (e.g. water and or lignin component fraction) of said fluid composition will separate after said specified period of time. In some embodiments, however, said fluid may require gentle stirring, agitation, and/or re-circulation is required for maintaining said stability and/or pumpability. For the avoidance of doubt, this gentle stirring, agitation, and/or re-circulation is not high-shear mixing.

According to some embodiments, a fluid composition is provided, wherein the sulfur content of said fluid composition is 3.0% (w/w) or less, for example 2.8% (w/w) or less, e.g. 2.6% (w/w) or less, for example 2.4% (w/w) or less, e.g. 2.2% (w/w) or less, such as 2.0% (w/w) or less, for example 1.8% (w/w) or less, such as 1.6% (w/w) or less, for example 1.4% (w/w) or less, e.g. 1.2% (w/w) or less, such as 1.0% (w/w) or less, for example 0.8% (w/w) or less, such as 0.4% (w/w) or less, such as 0.2% (w/w) or less, for example 0.1% (w/w) or less, such as 0.08% (w/w) or less, e.g. 0.06% (w/w) or less, such as 0.04% (w/w) or less. The above stated low sulfur contents of the fluid composition according to the first aspect of the present invention makes the fluid composition suitable for use as an environmentally friendly fuel.

### Second Aspect

In a second aspect, the present invention relates, in the broadest sense, to processes related to the fluid composition according to the first aspect of the invention, such as methods and processes for the manufacture of a fluid composition according to the first aspect of the present invention. Such a process according to the second aspect may comprise the steps of:
i. providing a fraction, preferably a solid fraction comprising a lignin component;
ii. providing an organic compound to make up at least part of said liquid organic fraction;
iii. intermixing the fraction provided in step (i) with the organic compound and/or liquid organic fraction provided in step (ii).

In one embodiment, the lignin component in step (i) is a “non-Kraft” lignin with features as e.g. described above, including lower LIEC, lower hygroscopy, and/or lower swelling.

In one embodiment of the second aspect of the present invention, the lignin component originates from a lignocellulosic biomass, which has been subjected to a hydrothermal pretreatment followed by a hydrolysis of at least part of the cellulose and at least part of said hemicellulose present in said lignocellulosic biomass. In a further embodiment of the second aspect of the present invention, said lignin component originates from a lignocellulosic biomass which has been subjected to a hydrothermal pretreatment followed by a hydrolysis of at least part of the cellulose and at least part of said hemicellulose present in said lignocellulosic biomass, furthermore followed by a fermentation. In yet a further embodiment, the lignin component originates from a lignocellulosic biomass which has been subjected to a hydrothermal pretreatment followed by a hydrolysis of at least part of the cellulose and at least part of said hemicellulose present in said lignocellulosic biomass, optionally followed by fermentation and/or hydrolysis.

In a still further embodiment of the second aspect of the present invention, said lignin component is obtained by pressing said fibrous fraction obtained after subjecting said lignocellulosic biomass to said hydrothermal pretreatment followed by said hydrolysis.

In yet a further embodiment of the second aspect of the present invention, said pressing of said fibrous fraction is preceded by rinsing and/or washing of said fibrous fraction.

In a still further embodiment of the second aspect of the present invention, said lignin component is obtained by mechanically comminuting said pressed fibrous fraction to a desired extent.

In still yet a further embodiment, the water content of said lignin component may be controlled and/or reduced, e.g. by drying.

The above specified modes of providing the lignin component to be used in the process of the second aspect of the present invention has proven especially beneficial due to the characteristics of the resulting lignin component.

In one embodiment of the second aspect of the present invention, said lignin component is as defined above in respect of the first aspect of the present invention.

In one embodiment of the second aspect of the present invention, said organic substance of said liquid fraction is having characteristics as defined above in respect of the first aspect of the present invention.

In one embodiment of the second aspect of the present invention, the process further comprising admixing of an amount of water.

In one embodiment of the second aspect of the present invention, the process further comprising admixing of one or more further agent, such as a dispersing agent. In a further embodiment, said fur further agent is selected from the group comprising or consisting of one or more dispersing agent(s), surfactant(s), hydrotropic agent(s), emulsifier(s), preserving agent(s), and any combination thereof.

In one embodiment of the second aspect of the present invention said lignin component and said organic substance of said liquid fraction, and optionally said amount of water and optionally said dispersing agent may be mixed together using a mechanical stirrer. In one embodiment, the mixing and/or intermixing is performed using one or more mixing device(s), such as a mechanical stirrer, high shear mixer, and/or a pump. In one embodiment of the second aspect of the present invention said lignin component and an amount of water are separately mixed using a mechanical stirrer; and furthermore, said organic substance of said liquid fraction and an amount of water, optionally also said dispersing agent are separately mixed using a mechanical stirrer; wherein said separately mixed mixtures are mixed and stirred. In a further embodiment, a step of separately intermixing an amount of water and optionally one or more further agent(s) such as a dispersing agent and with (a) said lignin component, (b) said organic compound of said liquid organic fraction, and/or (c) said liquid organic fraction is provided, and optionally wherein said separately mixed mixtures are mixed and stirred.

### Third Aspect

In a third aspect, the present invention concerns, in the broadest sense, to processes for treatment of a lignocellulosic treatment comprising the step of converting at least part of the a lignin component obtained in said process to a fluid composition, such as a fluid composition according to the first aspect of the invention, by admixing said lignin component with a liquid organic fraction comprising an organic compound or substance.

In one embodiment, a process for treatment of a lignocellulosic biomass is provided, wherein said process comprises:
a) subjecting said lignocellulosic biomass for hydrothermal pretreatment resulting in a hydrothermally pretreated lignocellulosic biomass;
b) subjecting at least part of said hydrothermally pretreated lignocellulosic biomass obtained in step (a) to a hydrolysis resulting in a liquid fraction comprising soluble carbohydrates, and a fiber fraction comprising a lignin component;
c) optionally subjecting at least part of the liquid fraction obtained in step (b) to a fermentation in order to ferment at least part of said soluble carbohydrates to a fermentation product, such as ethanol, methane or butanol, thereby obtaining a fermentation broth;
d) optionally isolating at least part of said fermentation product from the fermentation broth obtained in step (c) e.g. by distillation;
e) isolating at least part of the lignin component from one or more of: the fiber fraction obtained in step (b); the fermentation broth obtained in step (c); or after isolation of at least a part of the fermentation product in step (d);
f) converting at least part of the lignin component obtained in step (e) to a fluid composition by admixing said lignin component with a liquid organic fraction comprising an organic compound or substance.

Step a)-e) above represent the process of biorefining of a lignocellulosic biomass. This process has itself proven highly beneficial in converting waste biomass into a useful fuel, such as ethanol. It is believed that methods that do not comprise an alkaline treatment step, are beneficial. It is further believed that biorefining methods as outlined above, regardless if e.g. acid, such as H₂SO₄ or the like are added under pretreatment or not, provide a lignin or lignin component suitable for providing a fluid composition according to the first aspect of the invention. This may include methods comprising a “C5 bypass” or “C5 drain”, in e.g. a two-step pretreatment, wherein a liquid fraction rich in C5 sugars is collected after a first pretreatment step, e.g. by pressing (see e.g. WO2014/019589).

Here, step f) is added to this process making the former known process even more advantageous in that yet another renewable fluid and/or liquid energy product is obtained in the process. Thus in some embodiments, the fluid composition obtained in step (f) is a fluid composition according to any one of the preceding claims.

In a further embodiment relating to said process for treatment of a lignocellulosic biomass, said process comprises that at least part of said lignin fraction is isolated from the fiber fraction obtained in step (b).

In another embodiment relating to said process for treatment of a lignocellulosic biomass, said process comprises that at least part of said lignin fraction is isolated from said fermentation broth obtained in step (c).

In yet another embodiment relating to said process for treatment of a lignocellulosic biomass, said process comprises that said lignin component is obtained in step (e) by removing an associated liquid phase by using one or more separation device(s), such as a hydraulic press, a vacuum filtration unit, a belt filter, a rotary filter or a centrifuge decanter.

In yet a further embodiment relating to said process for treatment of a lignocellulosic biomass, said process comprises that said lignin component obtained in step (e) is dried to a residual water content at 110° C. of 2-20% (w/w), such as 4-18% (w/w), for example 6-16% (w/w), such as 8-14% (w/w), e.g. 10-12% (w/w). Alternatively, the lignin component can be dried at 105° C. or lower, such as a low as 50° C. Lower temperatures may require a longer drying period. Furthermore, the risk of e.g. microbial contamination and/or growth is increased when drying at lower temperatures. In one embodiment of the invention, the lignin/lignin component is dried at a temperature in the range of 50-150° C.

It is believed that drying the lignin component can be beneficial in terms of obtaining an improved suitable fluid composition, e.g. less viscous and/or more stable, for example, when the lignin/lignin component is, or is derived from a wet filter cake. A ‘dry lignin’ or ‘dry lignin component’ possesses usually less than 20% (w/w) water, preferably around or less than 15 or 10% (w/w) water. In some embodiments, the residual water content is as low as 5% (w/w) or lower, such as in the range of 0-5% (w/w) water, i.e. around 0.0, 0.5, 1.0, 1.5, 2.0, 2.5, 3.0, 3.5, 4.0, 4.5, or 5.0% (w/w). Lignin pellets and/or lignin granulate would usually qualify as ‘dry lignin’ or ‘dry lignin component’ as defined above.

In still a further embodiment relating to said process for treatment of a lignocellulosic biomass, said hydrothermal pretreatment of said lignocellulosic biomass is performed at a temperature of 150-260° C., such as 160-250° C., e.g. 170-240° C., such as 180-230° C., for example 190-220° C., such as 200-210° C.

In still another embodiment relating to said process for treatment of a lignocellulosic biomass, said hydrothermal pretreatment of said lignocellulosic biomass is performed in a period of residence time of 2-120 min., such as 5-110 min., e.g. 10-100 min., for example 15-90 min., such as 20-80 min., such as 25-70 min., e.g. 30-60 min, such as 35-50 min, such as 40-45 min.

In another embodiment relating to said process for treatment of a lignocellulosic biomass, said hydrothermal pretreatment of said lignocellulosic biomass is performed by subjecting said lignocellulosic biomass to a log severity, log(Rₒ) of 2.5 or more, such as a log(Rₒ) of 2.6 or more, e.g. a log(Rₒ) of 2.7 or more, such as a log(Rₒ) of 2.8 or more, for example a log(Rₒ) of 2.9 or more, such as a log(Rₒ) of 3.0 or more, such as a log(Rₒ) of 3.1 or more, for example a log(Rₒ) of 3.2 or more, e.g. a log(Rₒ) of 3.3 or more, such as a log(Rₒ) of 3.4 or more, such as a log(Rₒ) of 3.5 or more; such as a log(Rₒ) of 3.6 or more; for example such as a log(Rₒ) of 3.7 or more, e.g. a log(Rₒ) of 3.8 or more, for example a log(Rₒ) of 3.9 or more, for example a log(Rₒ) of 4.0 or more, such as a log(Rₒ) of 4.1 or more, or a log(Rₒ) of 4.2 or more; wherein the log severity is defined as: log(Rₒ)=(residence time)×(exp[Temperature−100/14.75]), and where residence time is measured in minutes and temperature in ° C.

From the pulp and paper industry, it has been understood that the extent of hemicellulose and lignin released to the aqueous phase was a function of the temperature to which the lignocellulose was heated and also of the residence time of the lignocellulose at the actual temperature. A person skilled in the art regularly make use of a “generalized severity parameter” which has shown to provide good comparisons of results obtained using different temperature and time protocols. C f. Abatzoglou et al., Chemical Engineering Science, Vol. 47, No. 5, p 1109-1122, (1992), the teaching of which is hereby incorporated by reference in entirety.

In another embodiments relating to said process for treatment of a lignocellulosic biomass, said hydrolysis is an acid catalyzed hydrolysis and/or enzymatic hydrolysis. In further embodiments, said hydrolysis is performed by one or more cellulases, such as by contacting said pre-treated biomass with one or more cellulases, and/or other enzymes, usually commercially available enzyme compositions developed for this specific type of applications. In yet a further embodiment, said one or more cellulases are selected from the group comprising exo-glucanases, endo-glucanases, hemi-cellulases and beta-glucosidases. In still a further embodiment, said hydrolysis is performed for a period of time of 1-200 hours, such as 5-190 hours, such as 10-185 hours, e.g. 15-180 hours, for example 20-175 hours, such as 25-170 hours, such as 30-165 hours, e.g. 35-160 hours, for example 40-155 hours, such as 45-150 hours, such as 50-145 hours, e.g. 55-140 hours, for example 60-135 hours, such as 65-130 hours, such as 70-125 hours, e.g. 75-120 hours, for example 80-115 hours, such as 85-110 hours, such as 90-105 hours, e.g. 95-100 hours.

In another embodiment relating to said process for treatment of a lignocellulosic biomass, said said step (b) and step (c) are performed as a separate hydrolysis and fermentation step (SHF), and wherein said hydrolysis is performed at a temperature of 30-72° C., such as 32-70° C., e.g. 34-68° C., for example 36-66° C., such as 38-64° C., e.g. 40-62° C., 42-60° C., e.g. 44-58° C., for example 46-56° C., such as 48-54° C., e.g. 50-52° C.

In another embodiment relating to said process for treatment of a lignocellulosic biomass, said said hydrolysis is performed in a period of time of 70-125 hours, e.g. 75-120 hours, for example 80-115 hours, such as 85-110 hours, such as 90-105 hours, e.g. 95-100 hours.

In another embodiment relating to said process for treatment of a lignocellulosic biomass, said step (b) and step (c) are performed as a simultaneous saccharification and fermentation step (SSF), and wherein said hydrolysis is performed at a temperature of 30-72° C., such as 32-70° C., e.g. 34-68° C., for example 36-66° C., such as 38-64° C., e.g. 40-62° C., 42-60° C., e.g. 44-58° C., for example 46-56° C., such as 48-54° C., e.g. 50-52° C.

In another embodiment relating to said process for treatment of a lignocellulosic biomass, said hydrolysis is performed in a period of time of 1-12 hours, such as 2-11 hours, for example 3-10 hours, such as 4-9 hours, e.g. 5-8 hours, such as 6-7 hours.

In another embodiment relating to said process for treatment of a lignocellulosic biomass, said step (b) and step (c) are performed as a simultaneous saccharification and fermentation step (SSF), and wherein said fermentation is performed at a temperature of 25-40° C., such as 26-39° C., e.g. 27-38° C., for example 28-37° C., e.g. 29-36° C., for example 30-35° C., such as 31-34° C. or 32-33° C.

In another embodiment relating to said process for treatment of a lignocellulosic biomass, said fermentation is performed in a period of time of 100-200 hours, such as 105-190 hours, such as 110-185 hours, e.g. 115-180 hours, for example 120-175 hours, such as 125-170 hours, such as 130-165 hours, e.g. 135-160 hours, for example 140-155 hours, such as 145-150 hours.

In another embodiment relating to said process for treatment of a lignocellulosic biomass, said lignin fraction obtained in step e) is converted to a fluid composition by admixing said lignin fraction with one or more organic substance and/or composition, said organic substance and/or composition constituting a liquid fraction.

In another embodiment relating to said process for treatment of a lignocellulosic biomass, said lignin fraction obtained in step e) is converted to a fluid composition by admixing said lignin fraction with an organic substance and with water, said organic substance constituting a liquid fraction

In another embodiment relating to said process for treatment of a lignocellulosic biomass, said lignin fraction obtained in step e) is converted to a fluid composition by admixing said lignin fraction with an organic substance, with water, and with one or more further agent, such as a dispersing agent, said organic substance constituting a liquid fraction.

The above modes of manufacture of the fluid composition according to the second and/or third aspect of the present invention have proved convenient and effective.

### Fourth Aspect

In a fourth aspect, the present invention relates to uses of a fluid composition according to the first aspect of the present invention, including a fluid composition provided according to the second or third aspect of the present invention.

The following embodiments relate to the use of the fluid composition as fuel. Interestingly, lignin itself represent a fairly high heating value but has the disadvantage of being a solid. Therefore, it is highly beneficial to be able to convert such a solid fuel to a liquid fuel which allows easier handling and storing and transportation of the fuel.

In one embodiment of the fourth aspect of the present invention the fluid composition is used as a fuel for a household burner.

In one embodiment of the fourth aspect of the present invention the fluid composition is used as a fuel for a boiler in a district heat plant or in a combined heat and power (CHP) plant.

In one embodiment of the fourth aspect of the present invention the fluid composition is used as a fuel for producing steam or other thermal energy products in an industry or factory using such steam or other thermal energy products to power its power consuming facilities.

In one embodiment of the fourth aspect of the present invention the fluid composition is used as a fuel in a boiler in a power plant.

In one embodiment of the fourth aspect of the present invention the fluid composition is used as a fuel in a start-up situation in a boiler in a power plant.

In a start-up situation in a power plant it is not possible to use solid fuel, such as coal due to the risk of dust explosions. Hence, for the time being in start-up situations power plants use fuel oil. The fuel oil is rather expensive and may have a high S content and it will be most advantageous to be able to substitute a traditionally and conventionally liquid fuel oil with a liquid fuel in the form of a fluid composition according to the first aspect or provided according to the second aspect of the present invention.

### Fifth Aspect

In a fifth aspect, the present invention relate to the use of lignin or a solid lignin component for a fluid composition, such as a fluid composition according to any of the previous aspects of the present invention. This includes also uses related to chemical processing of lignin and/or a lignin component or a conversion product thereof. The lignin and/or lignin component can e.g. be provided as described in the first, second, or third aspect of the invention.

According to one embodiment related to the fifth aspect of the present invention, said lignin and/or solid lignin component originates from a lignocellulosic biomass which has been subjected to a hydrothermal pretreatment followed by a hydrolysis.

According to one embodiment related to the fifth aspect of the present invention, said lignin component originates from a lignocellulosic biomass which has been subjected to a hydrothermal pretreatment followed by fermentation and/or distillation.

According to one embodiment related to the fifth aspect of the present invention, said hydrolysis is an acid catalyzed hydrolysis.

According to one embodiment related to the fifth aspect of the present invention, said hydrolysis is an enzymatic hydrolysis.

According to one embodiment related to the fifth aspect of the present invention, said hydrolysis comprises acid and enzymatic hydrolysis.

According to one embodiment related to the fifth aspect of the present invention, said fluid composition comprises a solid fraction and a liquid fraction.

According to one embodiment related to the fifth aspect of the present invention, said solid fraction and said liquid fraction are present in a state of being intermixed; said solid fraction comprises said lignin component; and said liquid fraction comprises an organic substance.

It is well-known in the art to use lignin or a lignin component as a feedstock for making organic chemicals, such as toluene.

However, handling of a solid feedstock in a chemical production plant may pose certain challenges as to the handling, transportation and storing of such solid material. Moreover, it is not easy to make a solid feedstock flow in a process line of a chemical manufacturing plant.

The invention of the fluid composition according to the first, second and/or third aspect of the present invention makes it possible to perform a chemical processing of a lignin component as a fluid or comprised in a fluid. This feature is utilized in the fifth_aspect of the present invention. Thus, in different embodiments of the fifth aspect of the present invention, the chemical processing of a lignin component may relate to one or more of the following:
a catalytic processing of said lignin component or a conversion product thereof
a non-catalytic processing of said lignin component or a conversion product thereof
an acid and/or base reactions of said lignin component or a conversion product thereof
an oxidation reaction of said lignin component or a conversion product thereof
a reduction reaction of said lignin component or a conversion product thereof
a hydrolysis reaction of said lignin component or a conversion product thereof
a pyrolysis of said lignin component or a conversion product thereof.
a hydrothermal conversion of said lignin component or a conversion product thereof
a supercritical fluid conversion of said lignin component or a conversion product thereof, such as a conversion involving water, methanol, and/or ethanol at supercritical conditions.
hydrogenation of said lignin component or a conversion product thereof
hydrodesulfurization of said lignin component or a conversion product thereof
hydrodenitrogenation of said lignin component or a conversion product thereof
processing involves hydrodeoxygenation and/or hydrogenation of said lignin component or a conversion product thereof.
processing involves hydrocracking of said lignin component or a conversion product thereof.
processing involves hydrodenitrification of said lignin component or a conversion product thereof.
oxidation of said lignin component or a conversion product thereof
cracking of said lignin component of said lignin component or a conversion product thereof, such as a technical cracking of said lignin component or a conversion product thereof; or a catalytical cracking of said lignin component or a conversion product thereof.

In the context of the present invention, the term “conversion product thereof” is meant to comprise the following: One may contemplate situations in which a lignin component is converted to a fluid composition according to e.g. the first aspect of the present invention. This fluid composition may be used for a chemical processing of a lignin component thus leading to reaction products of said chemical processing. However, the reaction product—still being in a fluid mixture—may itself be subject to further chemical processing of the same kind or of another kind. Many of such serial processing steps may be performed starting with a lignin component, leading to a first reaction product in a first chemical processing reaction. This first reaction product may be processed further to a second reaction product in a second chemical processing reaction and so forth.

In one embodiment of the fifth aspect of the present invention the said composition is a fluid composition according to any embodiment of the first aspect of the present invention as described above.

It has surprisingly turned out that a lignin component originating from a lignocellulosic biomass which has been subjected to a hydrothermal pretreatment followed by a hydrolysis may form stable fluids when mixed with a liquid fraction comprising an organic substance, and further impart beneficial properties to such fluids, such as e.g. a relatively low sulfur content and a relatively low viscosity.

Accordingly, the above details relating to the manufacture of such a lignin component are advantageous.

Further embodiments relating to the present invention are presented in the following section.

### EXAMPLES

Unless indicated otherwise in the Examples “%” is to be understood as “% (w/w)”. In some examples, the term “lignomulsion” is used for fluid compositions comprising lignin and/or a lignin component, such as for fluid compositions according to the various aspect of the present invention. Generally, and except when indicated otherwise, the lignin/lignin component samples used in this section were obtained from a second generation (2G) bioethanol manufacturing plant subjecting wheat straw to a hydrothermal pretreatment followed by an enzymatic hydrolysis, usually without addition of acids under pretreatment.

### Example 1

This example illustrates a preliminary experiment relating to the manufacture of a fluid composition according to a first aspect of the present invention.

A lignin component obtained from a second generation bioethanol manufacturing plant subjecting wheat straw to a hydrothermal pretreatment followed by an enzymatic hydrolysis was comminuted, dried and grinded in order to obtain a powder. The lignin component had a dry matter content of 95-97%.

This lignin component was exposed to moisture by wetting in order to obtain a lignin component having a dry matter content of 65% so as to mimic the wet lignin component originally obtained in the manufacturing process. 144.06 g of this lignin component together with 19.66 g diesel and 35.26 g water and 1.0 g Lutensol AP 10 dispersing agent from BASF was used for this fluid composition.

In a separate container 35.26 g water, 19.66 g diesel oil and 1 g Lutensol AP 10 was homogenized using the Ultra Turrax high speed mixer mixing at 10,000 min′ for 5 min.

The lignin/water mixture was added to the homogenized diesel/water/dispersing agent mixture and homogenized at 10,000 min′ for 5 min using the Ultra Turrax mixer.

A stable, viscous, liquid substance was obtained. The viscosity of the resulting fluid composition was measured using a “Stresstech HR” apparatus from Reologica Instruments AB, Sweden, in “Cup and Bob CC25” configuration.

Measurements were performed at the shear rates: 50, 100, 150, 200 and 250 min⁻¹ at 25° C. The viscosity was measured at these shear rates to be 0.33 Pa·s, 0.19 Pa·s, 0.16 Pa·s, 0.14 Pa·s, and 0.12 Pa·s, respectively.

Over the one week observed, the resulting fluid composition showed to be stable without any significant separation of diesel, water or lignin.

### Example 2

This example illustrates a second preliminary experiment relating to the manufacture of a fluid composition according to the first aspect of the present invention.

Example 1 was repeated with the same ingredients in the same amounts with the exception that in example 2 all the ingredients were mixed together.

The resulting fluid composition resembled that of example 1 with respect to stability and viscosity.

### Example 3

This example illustrates a third preliminary experiment relating to the manufacture of a fluid composition according to the first aspect of the present invention.

Example 1 was repeated with the same ingredients in the same amounts with the exception that in example 3 no dispersing agent was used.

The resulting fluid composition resembled that of example 1 with respect to stability and viscosity.

The examples show that it is possible to obtain a stable fluid composition according to the first aspect of the present invention starting from a lignin component originating from a biorefinery of a lignocellulosic biomass and using diesel as the organic substance of the liquid fraction of the fluid.

Most surprisingly, the examples demonstrate that it is possible to obtain a stable fluid composition according to the first aspect of the present invention starting from a lignin component originating from a biorefinery of a lignocellulosic biomass and using diesel as the organic substance of the liquid fraction of the fluid and without any inclusion of a dispersing agent.

### Example 4

This example reports studies with Indulin-containing compositions reported in examples from U.S. Pat. No. 5,478,366.

205 g of Indulin AT™, 225 g water, 75 g diesel oil, and two two dispersing agents from BASF: 1.0 g Lutensol AP8 and 1.0 g Lutensol AP10, were used for this fluid composition.

In a container, 1.0 g Lutensol AP8, 1.0 g Lutensol AP10, 225 g water and 75 g diesel oil were homogenized with the Ultra Turrax mixer for 5 min at 10,000 min⁻¹. 205 g of Indulin AT™ were added to this mixture in five portions of approximately 40 g; after the addition of each portion the mixture was homogenized with the Ultraturrax for 1 min at 10,000 min⁻¹. After the last portion, the mixture was homogenized with the Ultraturrax for 10 min at 20,000 min⁻¹.

The product obtained solidified rapidly. A ball formed by manually rolling the sample remained 3-dimensionally stable for several hours.

Immediately after homogenizing, the substance was transferred to a Haake ViscoTester VT550 apparatus with a MV-DIN measuring geometry and viscosity was measured over time at shear rate 100 s⁻¹. After 29.4 minutes, viscosity reached 2.1 Pa s, which is the limit of the instrumental capacity, and the apparatus stopped. This duration is defined as the “Stability Index”.

205 g of a lignin component obtained from a second generation bioethanol manufacturing plant (2G lignin, same material as in Example 1), 225 g water, 75 g diesel oil, and two two dispersing agents from BASF: 1.0 g Lutensol AP8 and 1.0 g Lutensol AP10, were used for a similar fluid composition. The components were mixed as described above, and viscosity was measured. The measurement ran for more than 3000 minutes. The viscosity did not reach the limiting value of 2.1 Pa s, and the measurement was stopped manually.

205 g of Indulin AT™, 225 g water, 75 g diesel oil, two dispersing agents from BASF: 1.0 g Lutensol AP8 and 1.0 g Lutensol AP10, and 5-50 g glucose (supplied by Merck) were used for a similar fluid composition. The components were mixed as described above, and viscosity was measured. The measurement ran for 75.0-86.3 minutes, as seen in the table below, before reaching the limiting value of 2.1 Pa s, causing the measurement to stop.

Finally, Indulin AT™ was washed with 1 M HCl solution and filtered. The filter cake was washed with water, until pH of the filtrate was above 5. The filter cake was then dried. 205 g of this substance was then mixed with 225 g water, 75 g diesel oil, two dispersing agents from BASF: 1.0 g Lutensol AP8 and 1.0 g Lutensol AP10. The components were mixed as described above, and viscosity was measured. The measurement ran for 856 minutes, as seen in the table below, before reaching the limiting value of 2.1 Pa s, causing the measurement to stop.

All results are summarixed in the table below

| Experi- | Lignin | Lignin | Oil | Water | AP8 | AP10 | Glucose | Stability |
|---|---|---|---|---|---|---|---|---|
| ment | type | g | g | g | G | g | g | index* |
| 1 | Indulin | 205 | 75 | 225 | 1 | 1 | 0 | 29.4 |
| 2 | 2G | 205 | 75 | 225 | 1 | 1 | 0 | <3000 |
| | lignin | | | | | | | |
| 3 | Indulin | 205 | 75 | 225 | 1 | 1 | 5 | 75.4 |
| 4 | Indulin | 205 | 75 | 225 | 1 | 1 | 25 | 75.0 |
| 5 | Indulin | 205 | 75 | 225 | 1 | 1 | 50 | 86.3 |
| 6 | Indulin, | 205 | 75 | 225 | 1 | 1 | 0 | 856 |
| | acid | | | | | | | |
| | treat | | | | | | | |

As shown, the stability of fluid compositions including Indulin AT™ is much poorer than comparable fluid compositions comprising 2G lignin.

See Example 17 for further detail.

### Example 5

30 g of Indulin AT™ was mixed with 120 g of a 23 (w/w) % KCl solution. The solution washomogenized with the Ultra Turrax 30 s at ˜10,000 rpm. It was shaken for more than one hour at room temperature. The mixture was filtered and the filter cake was washed four times with 4×100 ml water. The filter cake was then dried at 50 C, and the contents of potassium and chloride was measured.

30 g of Alkali lignin (Aldrich, product 471003) and two 30 g samples of lignin obtained from a second generation bioethanol manufacturing plant (2G lignin, as in Example 1) were treated in the same way as above.

Before the KCl treatment, all samples had similar K levels of 0.1-0.2%. However, after KCl treatment, Indulin AT™ and the Alkali lignin from Aldrich had a much higher K content (1.84 and 1.30%, respectively), while the 2G lignin sample had significantly lower K content (0.136-0.145%).

This clearly demonstrates the enhanced ability of Kraft lignin to bind K, relative to Inbicon 2G lignin, caused by an increased number of hydrophilic, polar functional groups in Kraft lignin.

The number of these groups can be estimated by calculating the so-called Lignin Ion Exchange Capacity (LIEC), here defined as the number of moles of potassium bound to lignin per kilo sample (unit: mol K/kg sample). This parameter has been calculated and is also given in the table below:

| | K1 | Cl1 | K2 | Cl2 | LIEC |
|---|---|---|---|---|---|
| Short name | % | % | % | % | mol/kg |
| Indulin AT ™ | 0.2265 | 0.0143 | 1.8424 | 0.0213 | 0.471 |
| Alkali lignin | 0.1645 | 0.0082 | 1.2969 | 0.0193 | 0.332 |
| 2G lignin 1 | 0.2286 | 0.0361 | 0.5318 | 0.0178 | 0.136 |
| 2G lignin 2 | 0.1358 | 0.0383 | 0.5672 | 0.0147 | 0.145 |

The low LIEC of 2G lignin demonstrates its low polarity and low hydrophilicity, compared to Alkali lignin and Indulin AT™, is the reason for the high stability of 2G lignin (as demonstrated in Example 4).

### Example 6

Two different mixtures containing lignin (L) from a second generation bioethanol manufacturing plant (2G lignin, as in Example 1), diesel oil (O) and water W were prepared with the following mass percentages: L:O:W 38:30:32 and 48:20:32, also containing 0.5% sodium benzoate and 0.5% Lutensol AP10, supplied by BASF.

The first formulation (38:30:32) was sieved through mesh 0.5 mm. The second and third formulations (48:20:32 and 50:30:20) was sieved through mesh 1 mm.

Each fuel composition was placed in a 1-litre container, and using pressurized air (8 bar) it was ejected through a nozzle (either a flat jet nozzle or full cone nozzle) into a combustion chamber and ignited.

After the initial ignition, the fuel was burned independently with a stable flame.

### Example 7

A long range of different hydrotropic and surface active components were tested formulations containing 40 (w/w) % lignin (2G lignin, same as in Example 1), 20 (w/w) % diesel oil and 40 (w/w) % water. The additives were present in the concentration 0.1-1%. Many of additives were able to lower viscosity at shear rate 100 s⁻¹. The successful ones among the hydrotropes included lignosulphonate, Pluronic PE 6800¹, Sokalan PA 20 and PA40², Sokalan CP10 (provided by BASF), sodium benzoate, sodium p-toluenesulphonate, sodium benzoate, methylparaben, propyl paraben, glucose and butyldiglycol—¹ Block copolymers in which the central polypropylene glycol group is flanked by two polyethylene glycol groups.² Polyacrylates; their average K value (molar mass) is indicated by the numeric code.

The successful ones among the surface active compounds included the Lutensol AP, XP³, TO⁴ and ON⁵ series from BASF. ³ Alkyl polyethylene glycol ethers based on C10-Guerbet alcohol and ethylene oxide, manufactured by reacting the C10-alcohol with ethylene oxide in stoichiometric proportions. The numeric portion of the product name indicates the general degree of ethoxylation.⁴ RO(CH2CH2O)xH, where R=iso-C13H27 and x=3, 5, 6, 6.5, 7, 8, 10, 12, 15 or 20⁵ RO(CH2CH2O)xH, where R=saturated, synthetic, short-chain fatty alcohol, x=3, 5, 6, 6.5, 7, 8 or 11

Adding 0.5% of either sodium benzoate, methylparaben or propylparaben to a mixture containing 40 g diesel oil, 80 g water and 80 g 2G lignin was tested as possible preserving agent to avoid microbial activity. All three candidates showed no signs of microbial activity, even after several months of storage at room temperature.

Instead of diesel oil, different other oils were tested in mixtures containing 80 g lignin, 80 g water and 40 g oil (with the addition of 1 g Lutensol AP10 and 1 g sodium benzoate). The oils include rapeseed methyl ether (supplied by Emmelev), pyrolysis oil from pyrolysis of wood, unrefined palm oil, unrefined rapeseed oil and a mixture of diesel oil and heavy fuel oil. Measurements of these mixtures were performed at the shear rates: 50, 100, 150, 200 s⁻¹ at 25° C. The viscosity was measured to be in the range 0-0.3 Pa s.

Over the one week observed, the resulting fluid compositions were stable without any significant separation of diesel, water or lignin observable.

Pre-drying of 2G lignin appears advantageous. Preparing a mixture from lignin filter cake (with a dry matter content of 59%), composed of 90 g lignin, 30 g oil and 180 g water (with 1.5 g sodium benzoate and 1.5 g Lutensol AP10) resulted in a viscosity at 10 s⁻¹ of 0.67 Pa s. However, this is still well below the viscosity measured for fluids containing Indulin AT as the lignin source, and over one week observed, the resulting fluid composition showed to be stable without any significant separation of diesel, water or lignin.

A relation has been observed between the input energy of the ultra turrax (T25) used for homogenizing and the viscosity of the resulting fluid. The input energy is a combination of the duration and speed of the ultra turrax and was determined by measuring the power consumption of the ultra turrax. See Example 23 for further details.

### Example 8

Ignition and combustion properties of five different formulation droplets were tested under well-controlled conditions in high-temperature suspension-fired boilers. Samples were prepared with lignin from a second generation bioethanol manufacturing plant (2G lignin, as in Example 1), water, diesel oil, unrefined palm oil and fuel oil (according to the table below).

TABLE 1
Overview of sample compositions tested.

| DONG | 2G | | | Palm | Fuel |
|---|---|---|---|---|---|
| label | Lignin | Water | Diesel | oil | Oil |
| 001 | 45% | 38% | 16% | 0% | 0% |
| 002 | 45% | 38% | 0% | 16% | 0% |
| 003 | 32% | 57% | 10% | 0% | 0% |
| 004 | 52% | 47% | 0% | 0% | 0% |
| 005 | 45% | 38% | 8% | 0% | 8% |

Samples (10 mg) placed on a sample holder were inserted into the reactor while shielded by a quartz tube. The tube was subsequently removed and the sample conversion/behavior followed by a high speed camera.

Delay before ignition and pyrolysis time were measured at three different conditions:
A: 1200° C., 5.5 O₂ and v=1.6 m/s (standard)
B: 1200° C., 2.9 O₂ and v=1.6 m/s (low oxygen concentration)
C: 990° C., 5.5 O₂ and v=1.6 m/s (low temperature)

### Results are Summarized Below

| | A | | B | | C | |
|---|---|---|---|---|---|---|
| | Delay + | Pyrolysis | Delay + | Pyrolysis | Delay + | Pyrolysis |
| | ignition | (10 mg) | ignition | (10 mg) | ignition | (10 mg) |
| Label | [ms] | [ms] | [ms] | [ms] | [ms] | [ms] |
| Diesel | 60 ± 15 | 1290 ± 230 | — | — | — | — |
| Fuel oil | 125 ± 70 | 2490 ± 290 | 115 ± 55 | 2685 ± 200 | 410 ± 175 | 3670 ± 365 |
| 001 | 350 ± 245 | 4335 ± 330 | 380 ± 105 | 3945 ± 45 | 2870 ± 835 | 3840 ± 570 |
| 002 | 1055 ± 495 | 2780 ± 460 | — | — | 3135 ± 610 | 2960 ± 835 |
| 003 | 835 ± 450 | 3010 ± 245 | — | — | 4120 ± 1340 | 3055 ± 760 |
| 004 | 960 ± 580 | 2585 ± 600 | — | — | — | — |
| 005 | 565 ± 520 | 3505 ± 410 | — | — | — | — |

^{*1}Total time = Delay + ignition + pyrolysis

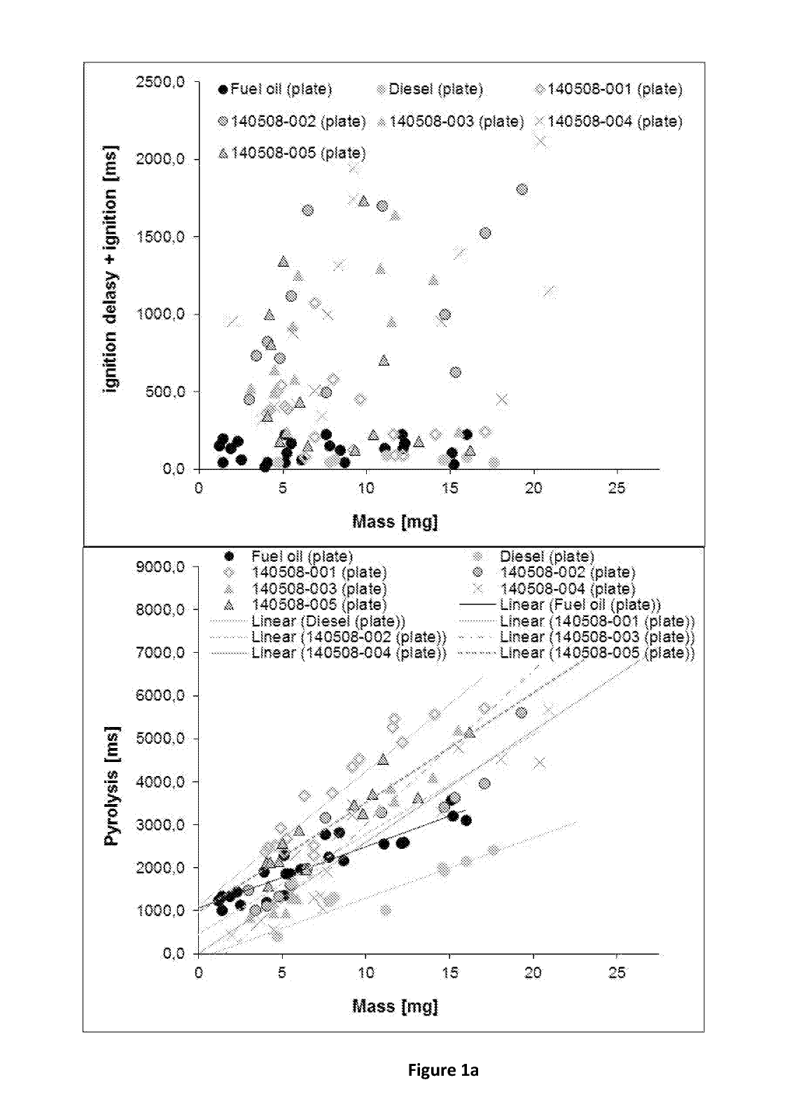

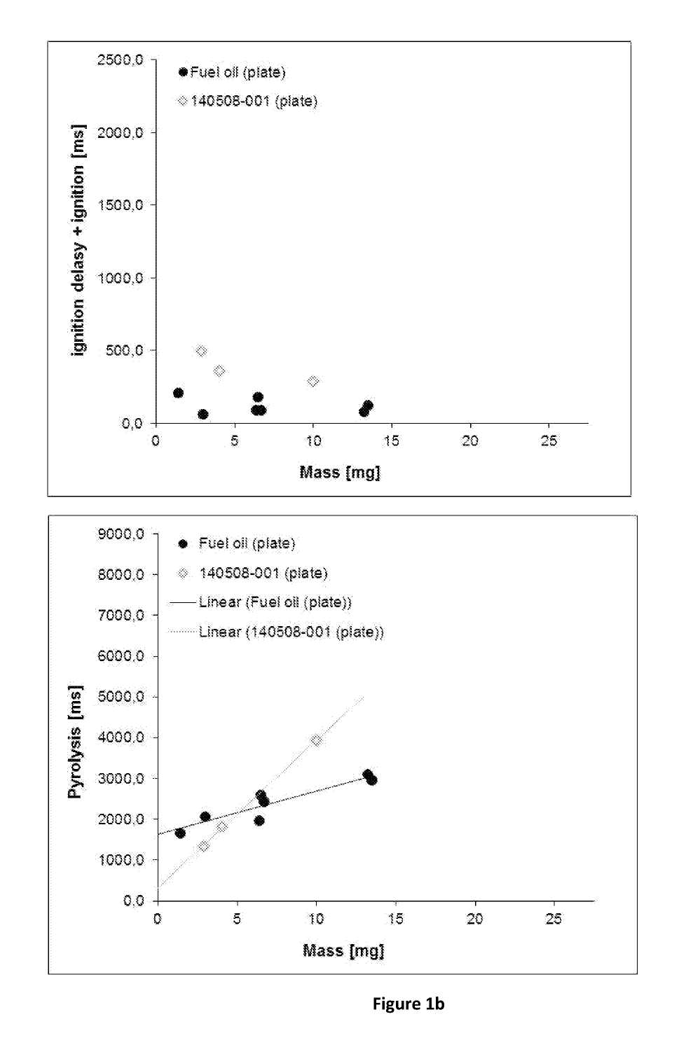

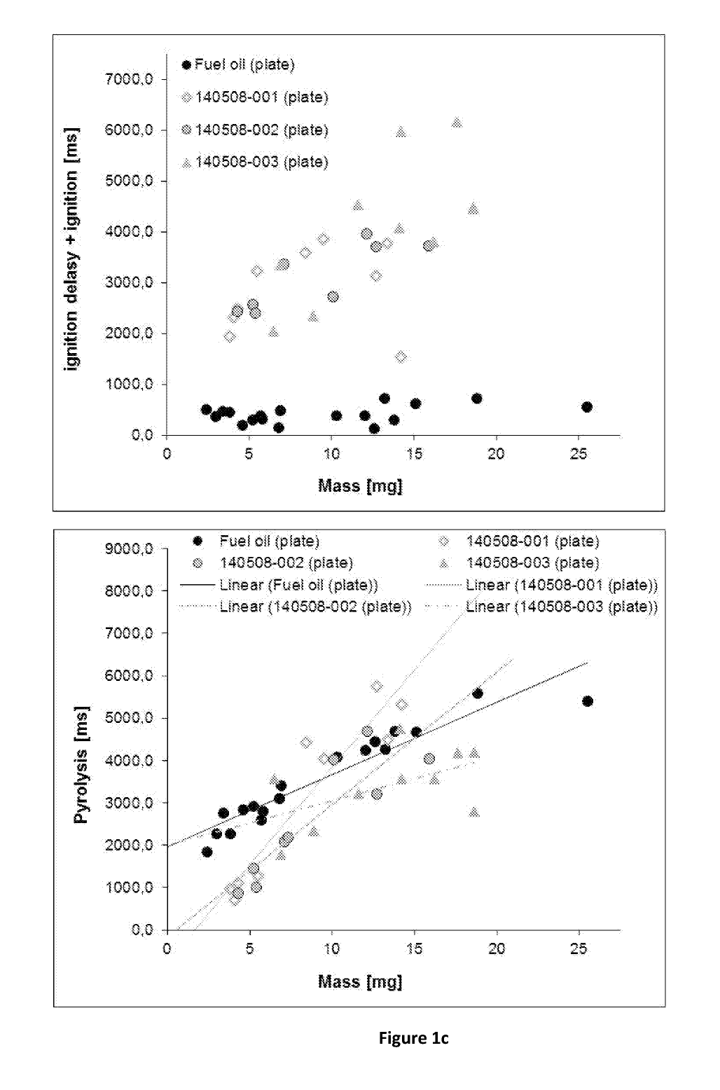

Different experiments are also summarized in FIG. 1.

Compared to fuel oil and diesel, the lignomulsion samples are associated with a longer ignition delay, relatively unaffected by a decreased oxygen concentration (from 5.5% to 2.9%), while it increases significantly with decreasing temperature. The difference in ignition behavior compared to fuel oil may be connected to water evaporation from the lignin samples.

No clear connection between sample mass and ignition delay/no stable flame is seen. The swiftest ignitions were observed for fuel oil, samples 001 and 005. Samples 002, 003 and 004 performs worst in terms of ignition delay, indicating a positive influence of diesel/oil and low water content.

Following the ignition phase, a stable flame is formed by combustion of pyrolysis gases. The pyrolysis time of 10 mg droplets (app 2.7 mm diameter) ranged from 2585 to 4335 ms at 1200° C. and 5.5% O₂, which is similar or slightly higher than the fuel oil (2490±290 ms). This is surprising, considering the lower heating value of the lignin slurry samples (9.7 to 15.4 MJ/kg) compared to fuel oil (40 Mj/kg).

The total conversion time (delay+ignition+pyrolysis) at 1200° C. and 5.5% 02 were generally higher for the lignin slurry samples compared to fuel oil.

### Example 9

### Materials and Experimental

Essentially, lignomulsion consists of three components: Lignin (L), Oil (O) and Water (W). Furthermore, additives capable of lowering viscosity and enhancing stability should be added. These components are all briefly described below

### 9.1 Lignin

Three different types of lignin were used: 1) Filter cake/centrifugate; 2) Dried, grinded lignin pellets (both supplied by Inbicon) and 3) Commercially available Kraft lignin.

As a standard case, dried lignin pellets were used.

### 9.1.1 Filter Cake

When lignomulsion is to be produced on large scale in the future, it appears advisable that the lignin source is provided through a 2G process, where lignin is isolated by pressing or centrifuging it to a dry matter (DM) content of 50-60%. Such material, referred to as filter cake, has been used in some experiments.

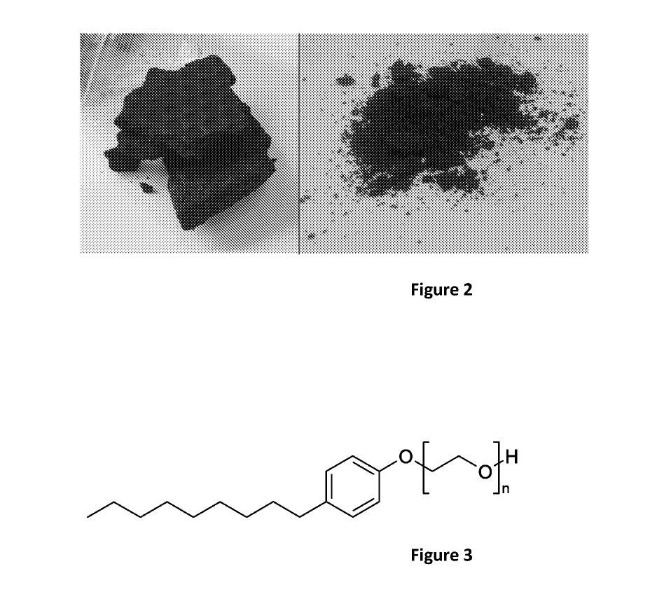

However, storage of large quantities of this material for laboratory test is difficult, as it should be in a freezer to avoid microbial activity. When using filter cake, this material was very tough and hard to break into pieces small enough for suspensions. However, the combination of the blade of a Kenwood machine and an Ultra Turrax did the job, at least on the scale of some kilo. Pictures of lignin filter cake before and after this treatment is shown in FIG. 2. Particle size of the cut material obtained in this way was studied. This is further described in the following.

Additionally, it should be mentioned that lignin from the 2G Demonstration plant at Kalundborg has sometimes not been pressed to a filter cake but is delivered as centrifugate with a DM of 30-40%. This was highly viscous and not pourable, in spite of the high water content, and appeared less suitable for lignomulsion.

### 9.1.2 Lignin Pellets

Lignin pellets with DM 95% is much less susceptible towards microbes and can be stored at room temperature. In a few case, microbial attacks on lignin pellets/granulate were observed, though.

Before use, the pellets were grinded to a fine powder (using large scale milling equipment) and sieved (mesh size ^{˜}150 μm).

### 9.1.3 Kraft Lignin

Two types of commercially available lignin were tested: Indulin AT (supplied by WestVaco) and Alkali Lignin (Sigma-Aldrich, item 249330).

### 9.2 Oil

Different oils have been used: Diesel oil (bought at Q8), heavy fuel oil (only used in a mixture with diesel), pyrolysis oil (from wood), biodiesels (from Emmelev and Daka), unrefined palm oil and rapeseed oil

### 9.3 Water

Cold tap water was used to prepare Lignomulsion

### 9.4 Additives

A range of additives were used; these can be divided into surfactants and hydrotropes. Surfactants and hydrotropes both contain a hydrophilic end and a hydrophobic end, allowing them to interact with both hydrophilic and hydrophobic compounds. In a water-oil emulsion, this increases the interactions between the two phases, enhances stability and lowers viscosity. The main difference between a surfactant and a hydrotrope is that in the hydrotrope, the contribution of the hydrophobic part is quite small, and the beneficial effects on stability and viscosity are more modest.

### 9.4.1 Surfactants

All tested surfactants were supplied by BASF. The preferred surfactants belonged to the class called Lutensol, ie ethoxylated nonylphenols, see an example FIG. 3.

### 9.4.2 Hydrotropes

Sodium benzoate was originally added to lignomulsion as a preserving agent, but testing showed it to have an effect on viscosity as well, identifying it as a hydrotrope. Several other hydrotropes (some commercially available, and some new products supplied by BASF) were tested.

9.5 Preparing emulsions

In general, emulsions were prepared by dissolving the hydrotrope in the water and mixing the surfactant in the oil phase (using the ultraturrax for some seconds). The two liquid phases when then mixed, and the lignin phase was added.

When using grinded, dry pellets, in most cases the lignin was first mixed with water in a 65:35 ratio, before the oil/water phases were added. When using Kraft lignin, all the liquid components were mixed, and lignin was added in small portions while using the Ultra Turrax. This was necessary to keep the viscosity down.

Viscosity was measured with a Haake ViscoTester VT550. Usually, the instrument was set to measure at shear rates 50, 100, 150 and 200 1 s⁻¹ and at the following temperatures: 298, 318, 338 and 358 K (25-85° C.). Viscosities were measured of different LOW formulations:
1. Formulations with different relative amounts of lignin, oil and water, either with or without the presence of a hydrotrope and/or surfactant.
2. Formulations where different additives (surfactants and hydrotropes) were tested.
3. Formulations with alternative preserving agents, ie parabens.
4. Formulations with different oils, including vegetable oil, biodiesel, pyrolysis oil and fuel oil
5. Formulations with Kraft lignin
6. Formulations with lignin from pellets compared to wet lignin filter cake
7. Formulations with lignin filter cake dried under different conditions
8. Formulations using different types of dried lignin pellets/granulate
9. Formulations using lignin with different sugar contents
10. Formulations with acid washed lignin
11. Formulations prepared with different ultra turrax energy input
12. Formulations stored for different periods of time

These experiments are further described in the following examples.

### Example 10

### Differences in Formulation

To test the importance on the relative contents of lignin, oil and water on viscosity, a number of different Lignomulsion formulations were prepared with a lignin content of 30-55%, and oil content of 0-30%, a water content of 30-55%, and either with or without 5000 ppm sodium benzoate and Lutensol AP10. The presence of these two additives influenced the resistance towards microbial activity as well as lowered viscosity and diminished phase separation.

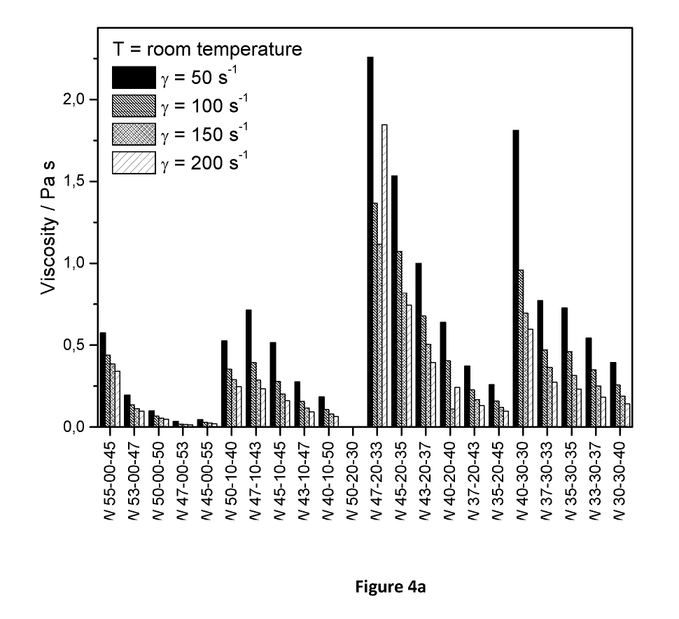

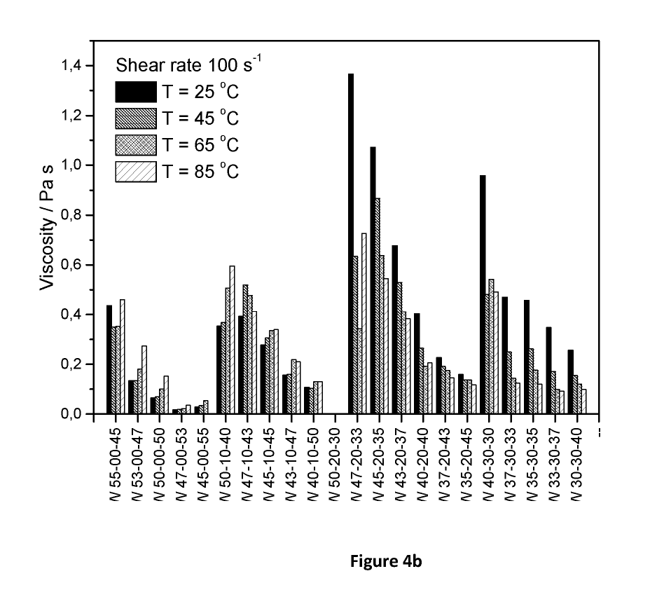

FIG. 4 shows viscosity of the formulations without additives measured at room temperature at the four different shear rates. Please note that viscosity of LOW 50-20-30 was not measured, since it was too viscous for the range of the available apparatus. In all formulations, viscosity decreases with increasing shear rate, showing Lignomulsion to be a non-Newtonian shear-thinning fluid.

The only exception is the LOW 47-20-33 formulation, which is highly viscous (>1 Pa s). The irregularities at high shear rate is therefore ascribed to uncertainties in the measurements.

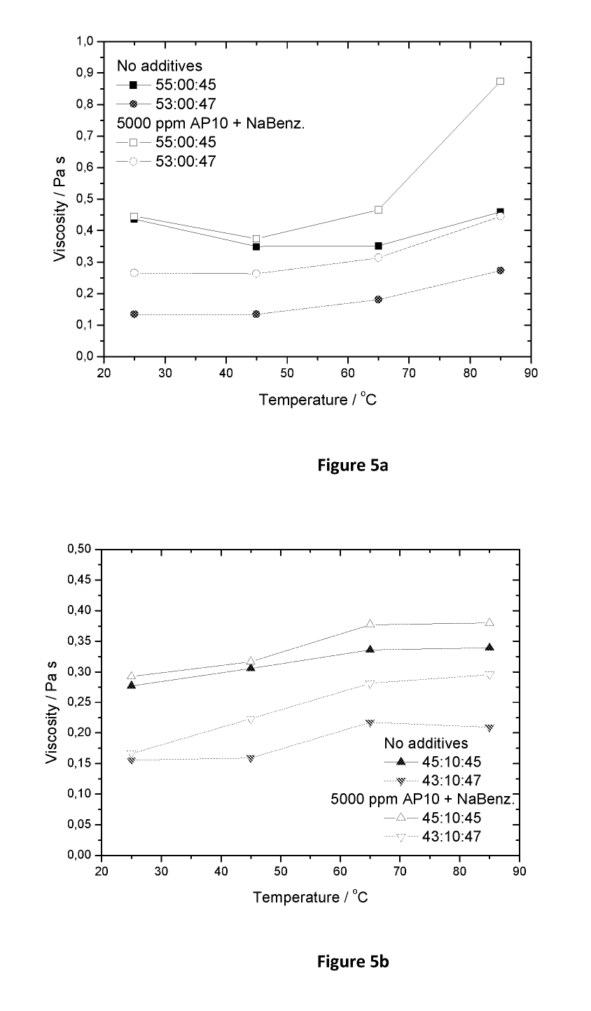

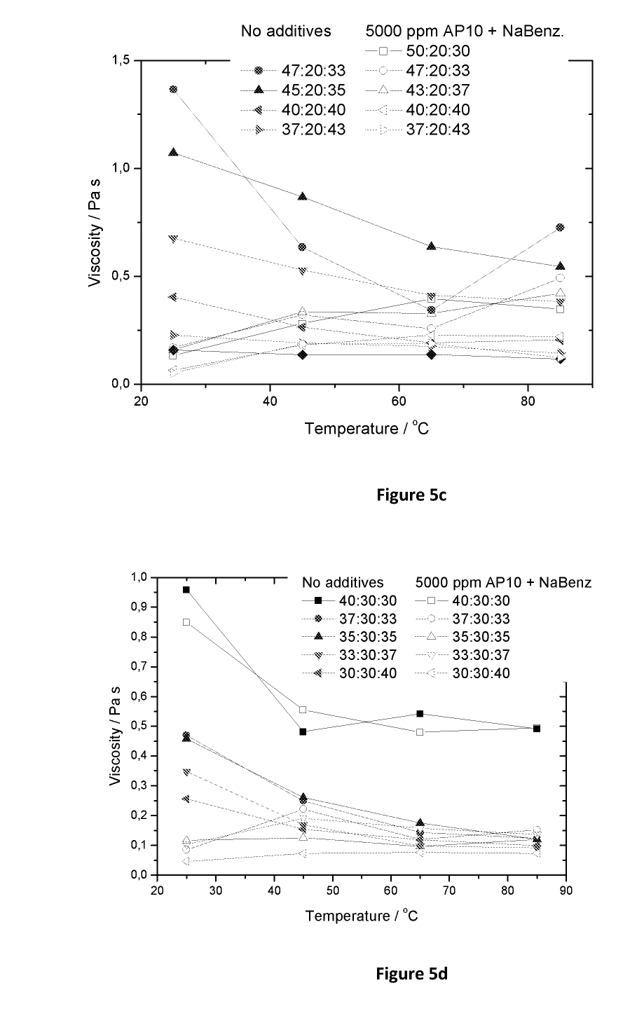

FIG. 5 shows viscosity of the formulations without additives measured at shear rate 100 s⁻¹ for four different temperatures. Different temperature effects are observed, depending on the formulation:

Formulations containing 20-30% oil show a decrease in viscosity with increasing temperature. This is the most common temperature behaviour in dispersions, suspensions and emulsion, as structuring elements are generally broken down and internal friction decreases at higher temperatures. However, for formulations with 0-10% oil viscosity increases with increasing temperature. When viscosity decrease, it could be an effect of intermolecular structures being built up in the dispersion, or it could be an effect of components of the liquid phase being absorbed by the dispersed phase. In this case, a possible explanation could be that water is taken up by the large structures of the lignin molecules. As lignin contains many hydrophobic groups, this process is facilitated by the presence of the additives, and it should also be enhanced by increasing temperature. The reason why this effect is only seen for small oil contents could be that oil can interfere with these lignin-water interactions, for example by physically or chemically “blocking” potential sites for hydrogen bonding.

In FIG. 5, the effect of additives is shown by comparing identical LOW formulations, either with or without sodium benzoate and Lutensol AP10. All measurements were performed at 100 s⁻¹.

For all emulsions with a low oil content (0-10%), the effect of additives was modest, as seen in FIGS. 5a and b. At room temperature, the additives had no significant effect on viscosity in the 10% oil case and even increased viscosity in the 0% oil case. These observations are also valid at shear rate 50, 150 and 200 s⁻¹ (data not shown). This is not surprising; the additives are aimed at optimizing the interactions between water and oil and obviously do not work in dispersions with no oil. In the dispersions with only 10% oil, it is likely that lignin—containing both hydrophobic and hydrophilic functional groups and therefore acting as a hydrotrope—was able to act as an adequate emulsifier.

One effect of additives in the low oil formulations is, however, that while increases in viscosity as a function of temperature were observed in formulations without additives, even higher increases are observed for formulations with additives. This observation is in full correspondence with hypothesis described above, since at high temperature lignin restructuring will expose more sites for hydrogen bonding, and the additives facilitates interactions between lignin and water.

For higher oil contents, the additives efficiently reduces viscosity, in some cases with more than 80% (for example LOWs 47:20:43 and 45:20:45 at room temperature). On the other hand, the slight increases in viscosities are observed with increasing temperature. This is contrary to the behaviour observed in emulsions with no additives, and it means that in many cases the viscosity lowering effect of the additives is matched by this temperature effect at 65-85° C. For emulsions with the lowest lignin content, temperature effects do not seem significant, but in the emulsions with the highest lignin contents (ie 40:30:30, 50:20:30 and 47:20:33) viscosity at high temperature is >0.3 Pa s, which would make pumping difficult.

Again, the explanation for the temperature effect is that water is transferred from the liquid phase to the lignin phase due to interactions with the hydrophilic groups of lignin. Higher temperature allows water to access more hydrophilic sites within the lignin structure, and while the high oil content may block or restrict some of these sites, the additives diminished this effect by increasing the favourable interactions between oil and water. Furthermore, the additives may even make it possible for oil to be bound to the hydrophilic groups of lignin—which further transfers mass from the liquid to the dispersed phase, also resulting a lowering of viscosity.

### Example 11

### Surfactants and Hydrotropes

In general, surfactants and hydrotropes facilitates interactions between lignin, water and oil making the emulsions less viscous. The favoured additives of lignomulsion include one hydrotrope and one surfactant. The initial choices were sodium benzoate and Lutensol AP10 (note that sodium benzoate was initially chosen because of its anti-microbial properties, with it hydrotropic properties as an added benefit), but several other additives were also tested.

First, the effects of adding AP10 and sodium benzoate in various amounts were evaluated by preparing formulations as shown in Table 11-1.

TABLE 11-1
Emulsions for studying the effect of additive concentration

| | | | Viscosity |
|---|---|---|---|
| | [AP 10] | [NaBenz] | (25° C.; 100 s⁻¹) |
| LOW | (w/w) % | (w/w) % | Pa s |
| 40-20-40 | 0 | 0 | 0.404 |
| 40-20-40 | 0.5 | 0 | 0.177 |
| 40-20-40 | 0.5 | 0.1 | 0.105 |
| 40-20-40 | 0.5 | 0.5 | 0.0650 |
| 40-20-40 | 0.5 | 1 | 0.0617 |
| 40-20-40 | 0 | 0.5 | 0.335 |
| 40-20-40 | 0.1 | 0.5 | 0.322 |
| 40-20-40 | 0.4 | 0.5 | 0.188 |
| 40-20-40 | 1 | 0.5 | 0.0575 |

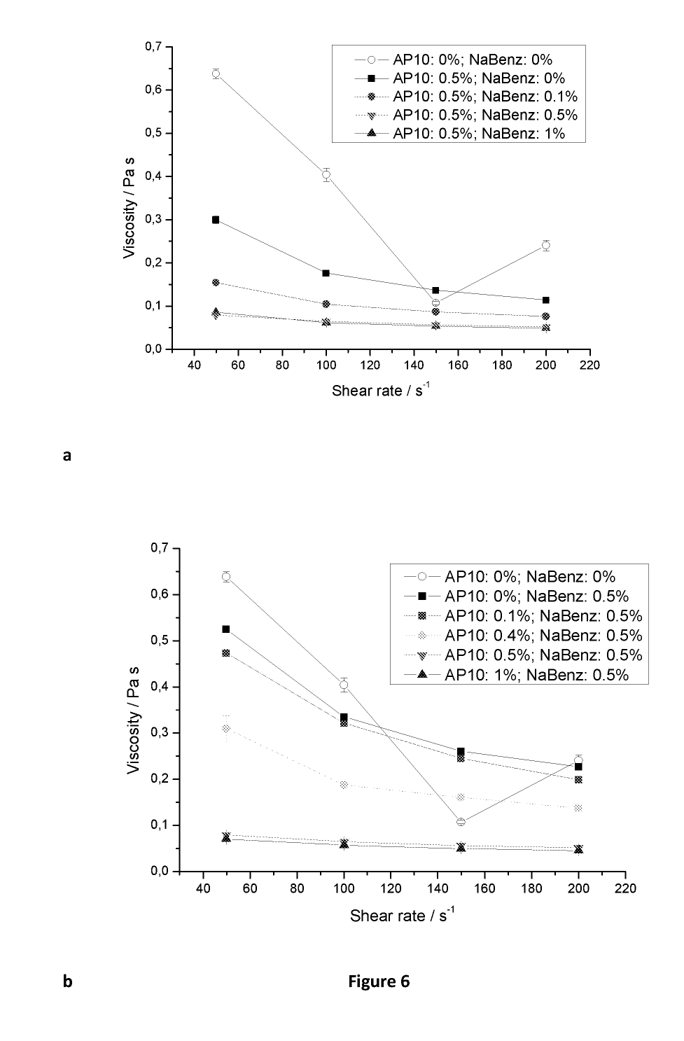

FIG. 6 shows how viscosity at room temperature and shear rate 50-200 s⁻¹ depends on the concentrations of sodium benzoate and Lutensol AP10, respectively, in a LOW 40-20-40 mixture. This formulation was chosen because additives were found to have large effect on formulations with at least 20% oil, and the formulation with 40% lignin resulted a thin, usable, pumpable liquid when additives were present while still having measurable viscosity without the additives.

Both additives were found to independently lower viscosity. At shear rate 100 s⁻¹, in the case of 5000 ppm sodium benzoate, a reduction of 17% (from 0.40 to 0.34 Pa s), and for 5000 ppm Lutensol AP10 a reduction of 56% (from 0.40 to 0.18 Pa s) were observed. However, in combination (ie 5000 ppm of each) sodium benzoate and Lutensol AP10 lower viscosity by 84% (from 0.4 to 0.065 Pa s), indicative of a synergistic effect between the two additives. There is a clear connection between the concentration of each additive and the decrease in viscosity, at least up to a concentration of 5000 ppm for the studied formulation of LOW 40-20-40.

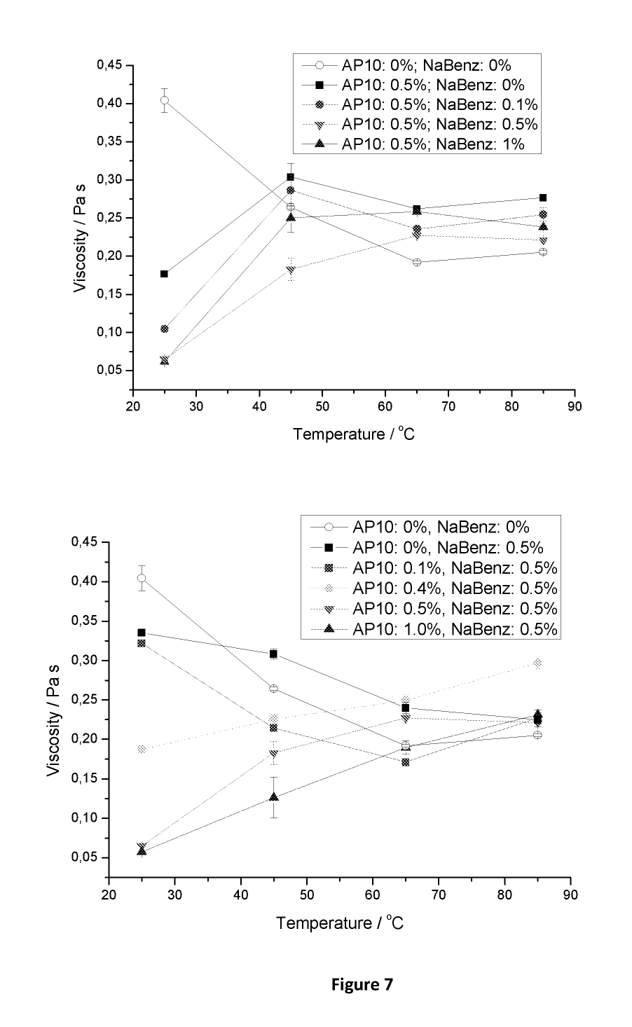

FIG. 7 shows how viscosity at shear rate 100 s⁻¹ and temperature 25-85° C. depends on the concentrations of sodium benzoate and Lutensol AP10, respectively, in a LOW 40-20-40 mixture. Adding 5000 ppm sodium benzoate and only 0 or 1000 ppm AP10 does not change the temperature effect observed in Example 10. For these formulations, viscosity decreases as a function of temperature—as was also observed for formulations with no additives present. It is therefore clearly Lutensol AP10 that is responsible for the increase in viscosity as a function of temperature observed for formulations with an oil content of 20-30%

It is also interesting to observe that while viscosity decreases with temperature when no additive is added, the opposite effect is observed when either one or both additives are present, and that this temperature become more pronounced when the amount of additive is increased.

A theory is that the 3D polymeric structure of lignin unfolds and expands when heated. This exposes more sites for H-bonding with water; this process is facilitated and strengthened by the hydrotrope or surfactant additives. Further experiments indicates that this process is to some extent irreversible, as the water partially remains trapped within the lignin structure, even after cooling to room temperature.

Since the viscosity increasing as a function of temperature is not a desired property, several other surfactants and hydrotropes were investigated. A long range of hydrotropes were obtained either from commercial sources (sodium xylanesulphonate, lignosulphonate, sokalan, sodium benzoate, sodium p-toluenesulfonate, BDG and glucose) or from other sources.

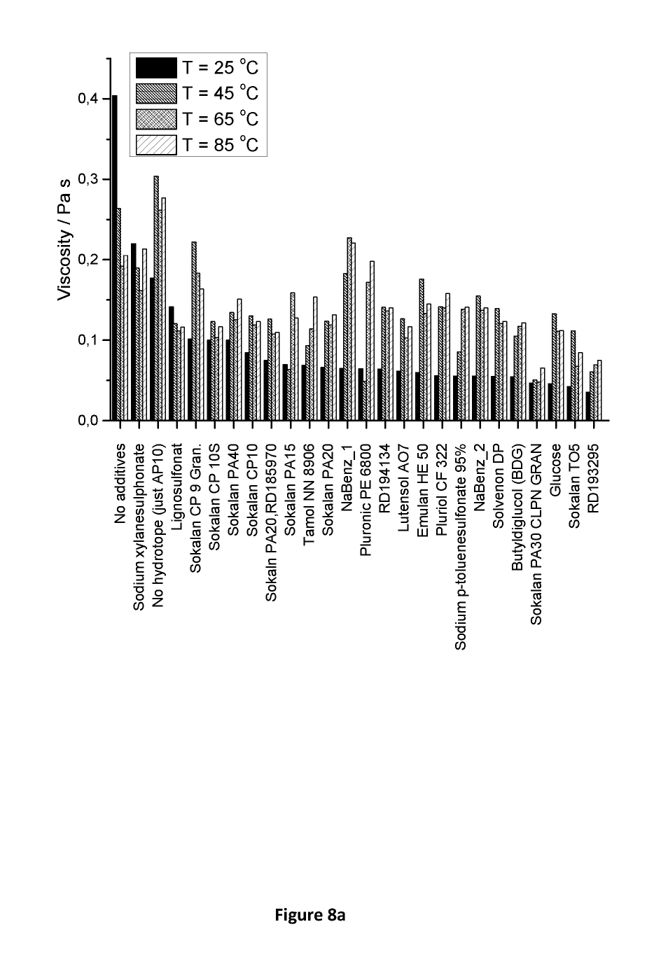

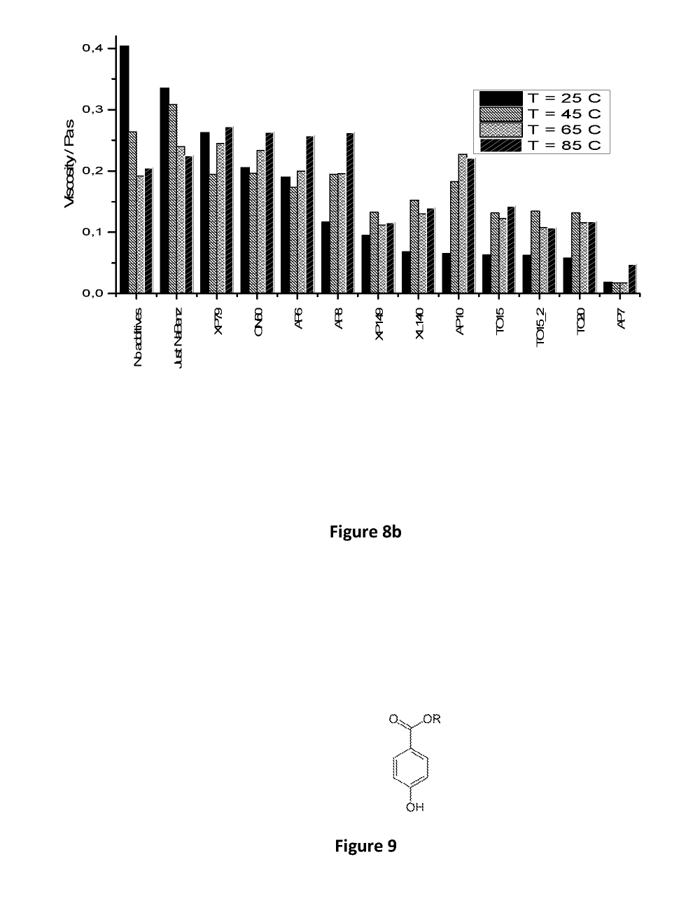

As seen in FIG. 8a, several hydrotropes effectively lower viscosity, but sodium benzoate has added advantage of being a preserving agent. FIG. 8b shows that several surfactant are as efficient at lowering viscosity as Lutensol AP10 and therefore, other choices could be made. Also, notice that for all efficient hydrotropes and surfactants, viscosity increases as a function of temperature.

### Example 12

### Parabens

Sodium benzoate is the preferred preserving agent in Lignomulsion, but parabens were studied as alternatives. A generic structure of a paraben is shown in FIG. 9; in this study-R was either methyl- or propyl.

The potential disadvantage with parabens (ie esters of parahydroxybenzoic acid) is that they are only antimicrobial in the acid form. Above pH 6 the acid is converted to a salt, which is totally inactive. As none of the components in Lignomulsion have acidic properties, this is problematic. Also, parabens function only in the water phase—if they are extracted to the oil phase of lignomulsion, they will become inactive.

In all formulations presented in this section, it was necessary to heat water to more than 60° C. and stir for several minutes before the entire amount had been dissolved. Hopefully, it did not precipitate when the other ingredients were added, but due to the dark colour of Lignomulsion there was no way to actually ascertain this.

The advantage is that parabens are active when used in much smaller quantities than sodium benzoate.

Two parabens, methyl- and propylparabene, were used to prepare six different formulations, as specified below in Table 12-1.

TABLE 12-1

| | Masses added/g | | | | | | | | | |
|---|---|---|---|---|---|---|---|---|---|---|
| | Parabene | | | | | Parabene | | | | |
| Parabene ID | amount | Lignin | Oil | W | AP10 | amount | Lignin | Oil | W | AP10 |
| Methylparabene | 0.05 | 40 | 20 | 40 | 0.5 | 0.1535 | 185.6 | 60 | 55 | 1.5 |
| Propylparabene | 0.05 | 40 | 20 | 40 | 0.5 | 0.1499 | 185 | 60 | 55 | 1.5 |
| Methylparabene | 0.01 | 40 | 20 | 40 | 0.5 | 0.0323 | 185.1 | 60 | 55 | 1.4951 |
| Propylparabene | 0.01 | 40 | 20 | 40 | 0.5 | 0.0319 | 185.5 | 59.9 | 55.8 | 1.4927 |
| Methylparabene | 0.005 | 40 | 20 | 40 | 0.5 | 0.0157 | 185.7 | 60.1 | 55.2 | 1.4988 |
| Propylparabene | 0.005 | 40 | 20 | 40 | 0.5 | 0.0149 | 185.2 | 60 | 55 | 1.4932 |

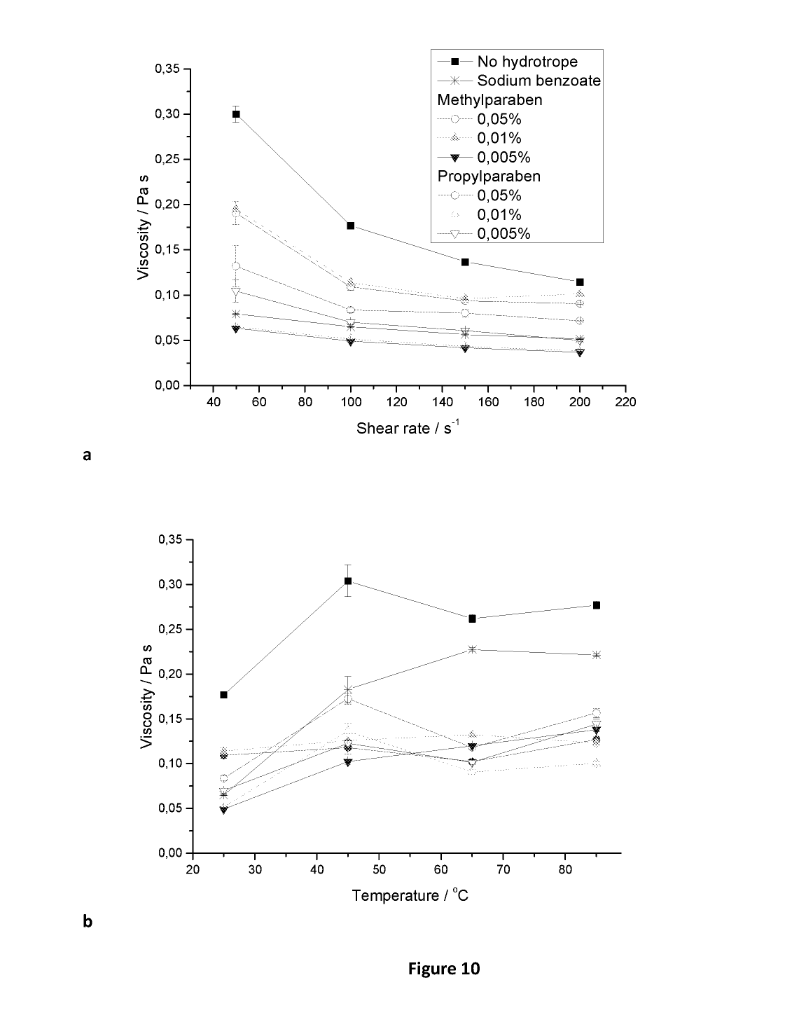

FIGS. 10 a-b shows that the presence of a paraben lowers viscosity; i.e. it acts as a hydrotrope, and at temperatures above room temperature, it is even more efficient than sodium benzoate (which, as was shown above, is among the most efficient hydrotropes). However, there does not seem to be a straightforward relation between the added paraben quantity and viscosity suppression. This could be due to the difficulty with dissolving paraben in water—possibly in some formulations, the entire amount of added parabens was not dissolved.

As a preserving agent, parabens also seem efficient in suppressing microbial activity.

### Example 13

### Different Oils

As a general case, diesel oil is used in Lignomulsion. However, other oils have been tested, including biodiesel (from either fish products or rapeseed) and vegetable oils (sunflower oil, unrefined palm or rapeseed oil and oil from wood pyrolysis), see Table 13-1. Other oils are believed to be suitable, too.

TABLE 13-1
Oils used in Lignomulsion formulations

| | Source/ | Sample | LOW | Viscosity** |
|---|---|---|---|---|
| Type | producer | ID | formulations* | Pa s |
| Fish diesel | Pronova | 131107_001 | 40-20-20 | 0.055 |
| (Fish) | biopharma | 131108_001 | 40-30-30 | 0.079 |
| Heavy bio diesel, | Daka | NA | 40-20-20 | NA |
| FAM (Daka) | | NA | 40-30-30 | NA |
| Rapeseed methyl | Emmelev | 131204_001 | 40-20-20 | 0.171 |
| ether (RME1) | | 131220_003 | 40-30-30 | 0.136 |
| Rapeseed methyl | Emmelev | 131205_001 | 40-20-20 | 0.231 |
| ether, secunda | | 131217_001 | 40-30-30 | 0.153 |
| (RME2) | | | | |
| Sunflower oil | | | 40-20-20 | |
| (Sunflower) | | 131104_001 | 40-30-30 | 0.648 |
| | | 130320_001 | 30-30-40 | 0.062 |
| Pyrolysis oil | B21st, WP4 | 131115_001 | 40-20-20 | 0.217 |
| (Pyro) | | 131115_002 | 40-30-30 | NA |
| Unrefined palm oil | Neste Oil | 140502_001 | 40-20-40 | |
| (Palm) | | 140505_001 | 40-30-30 | |
| | | 140505_002 | 45-15-40 | |
| Rapeseed oil | Neste Oil | NA | 40-20-40 | 0.046 |
| (RSO) | | NA | 40-30-30 | 0.158 |
| | | NA | 45-15-40 | NA |

*Formulations also contained 5000 ppm Lutensol AP10 and 5000 sodium benzoate
**Measured at room temperature and shear rate 100 s⁻¹

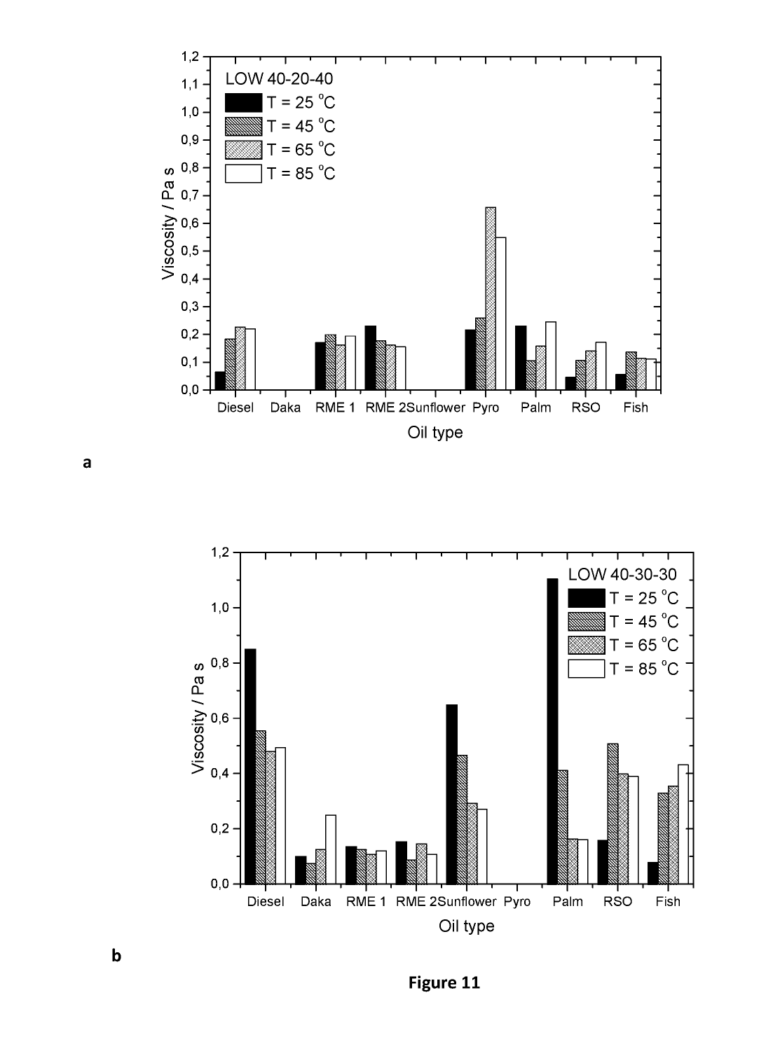

FIG. 11 shows viscosity measured at 100 s⁻¹ and four different temperature for the two LOW formulations 40-20-40 and 40-30-30 (with 5000 ppm sodium benzoate and 5000 ppm Lutensol AP10) using different oils. The viscosity of LOW 40-30-30 with pyroysis oil was too high to be measured by the Viscotester, so results for this formulation are not available. Pyrolysis oil has a low pH which can result in corrosion of tanks and equipment (as well as high cost), and using this oil was never preferable for large-scale lignomulsion production.

Unrefined palm oil has a melting point close to (or slightly above) room temperature, and is semi-solid at room temperature. It produces formulations which are quite viscous at room temperature. The decrease in viscosity from 25° C. and 45° C. observed in both of the displayed palm oil formulations is therefore partially due to a softening/melting of the oil.

The viscosities of formulations prepared with DAKA FAME and Emmelev RME1 and -2 do not change significantly with temperature, whereas viscosities of formulations prepared with unrefined rapeseed oil and the fish diesel increase with increasing temperature; similar to what was observed for conventional diesel oil.

Except for the pyrolysis oil and palm oil at room temperature, replacing conventional diesel with biodiesel or bio-oil seems perfectly possible from a viscosity point of view.

As unrefined palm oil or rapeseed oil has been suggested as the component in a “non-fossil” version of Lignomulsion, such formulations are studied further in 12, including a third formulation, LOW 45-15-40. The formulations with 40-45% lignin and 15-20% palm oil have viscosity ^{˜}0.3 Pa s at shear rate 100 s⁻¹ and room temperature, with viscosity decreasing slightly when temperature is increased to 45° C. (as mentioned above). When temperature increases further to 65 and 85° C., viscosity increases. This is most likely related to expansion of the lignin structure and subsequent water uptake, as also mentioned above.

In formulations with 40% lignin, increasing palm oil content from 20% to 30% results in a massive viscosity increase. However, viscosity decreases as a function of temperature to an extent that viscosity of the LOW 40-30-30 formulation is actually lower than the LOW 40-20-40 formulation at 65 and 85° C.

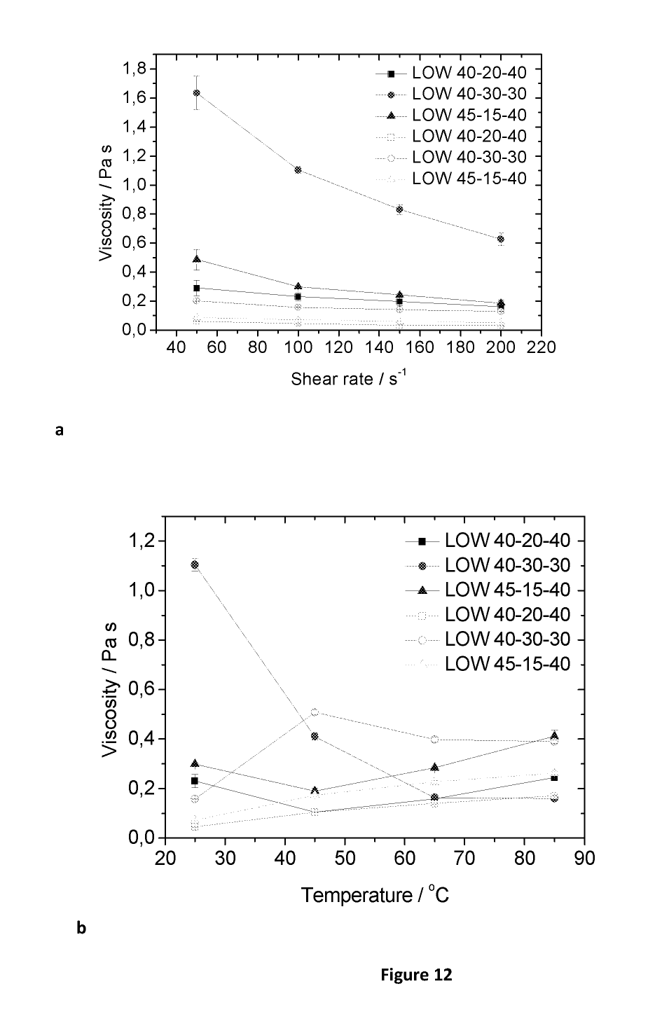

Similar quantitative behaviours are seen in FIG. 12 for the RSO formulations, except that RSO results in a much lower viscosities. This is probably because the RSO sample has a lower melting point than palm oil and is liquid at room temperature.

### Example 14

### Fuel Oil

In a series of experiments, Lignomulsion formulations with a mixture of diesel oil (DO) and heavy fuel oil (FO) were studied, see Table 13.

TABLE 14-1
Formulations of lignomulsion prepared with diesel and fuel oil

| | | | | Sodium | | |
|---|---|---|---|---|---|---|
| | Lignin | OD:FO | Water | benzoate | AP10 | Viscosity* |
| ID | % | % | % | Ppm | Ppm | Pa s |
| 130617_002 | 45 | 5:5 | 45 | 5000 | 5000 | 0.0549 |
| 130610_002 | 43 | 5:5 | 47 | 5000 | 5000 | 0.0578 |
| 130612_001 | 45 | 5.5:5.5 | 44 | 5000 | 5000 | 0.0728 |
| 130611_001 | 39 | 9:9 | 44 | 5000 | 5000 | 0.0391 |
| 130610_003 | 35 | 9:9 | 47 | 5000 | 5000 | 0.0420 |
| 130619_001 | 40 | 10:10 | 40 | 5000 | 5000 | 0.0575 |
| 130617_003 | 33 | 15:15 | 37 | 5000 | 5000 | 0.0537 |
| 130617_001 | 30 | 15:15 | 40 | 5000 | 5000 | 0.0338 |
| 130619_002 | 40 | 8:12 | 40 | 5000 | 5000 | 0.1143 |
| 130620_001 | 40 | 4:16 | 40 | 5000 | 5000 | 0.1642 |
| 130619_001 | 40 | 10:10 | 40 | 5000 | 5000 | 0.0575 |

*measured at room temperature and shear rate 100 s⁻¹

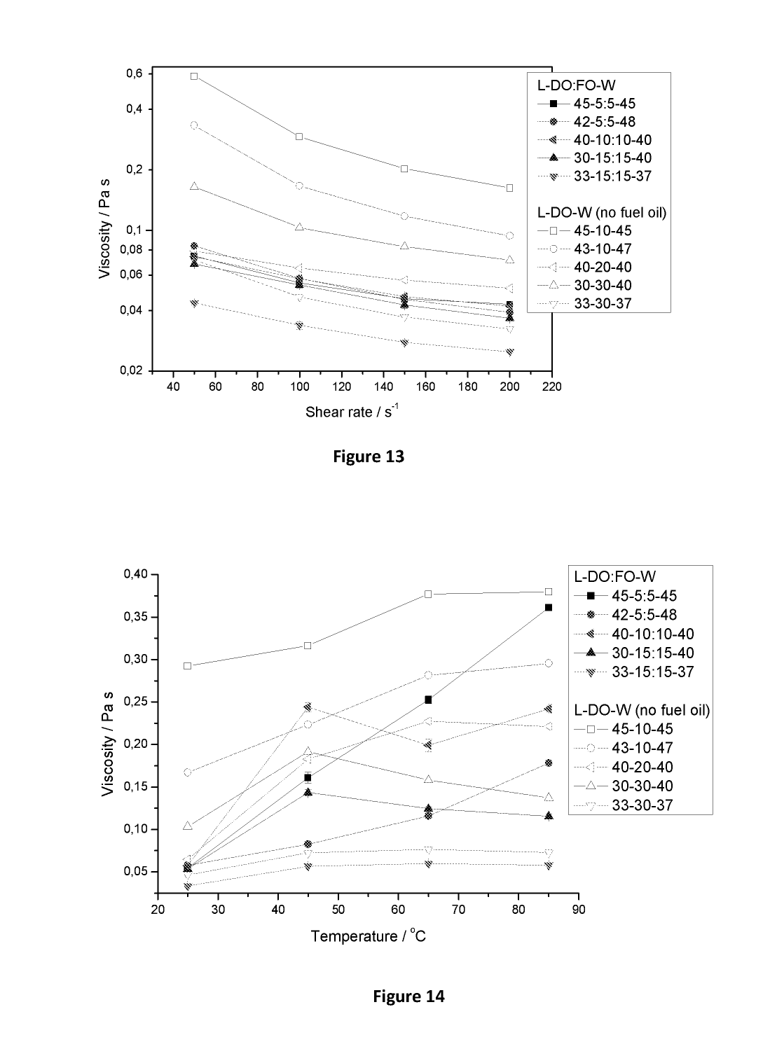

In FIGS. 13 and 14, viscosity as a function of shear rate and temperature, respectively are shown for Lignomulsion formulations prepared with a 1:1 mixture of diesel and fuel oil; these were compared to formulations containing only diesel oil. Note, that in FIG. 13, the y-axis is logarithmic to better distinguish between the different data sets.

Replacing half of the diesel oil with heavy fuel oil (which is a highly viscous substance) significantly lowers viscosity of the formulation at all shear rates and all temperatures. As the most extreme example, in LOW 45-10-45 at shear rate 100 s⁻¹ and room temperature, replacing half of the diesel with heavy fuel oil, results in a 80% reduction in viscosity. However, the viscosity reduction is smaller for the other formulations.

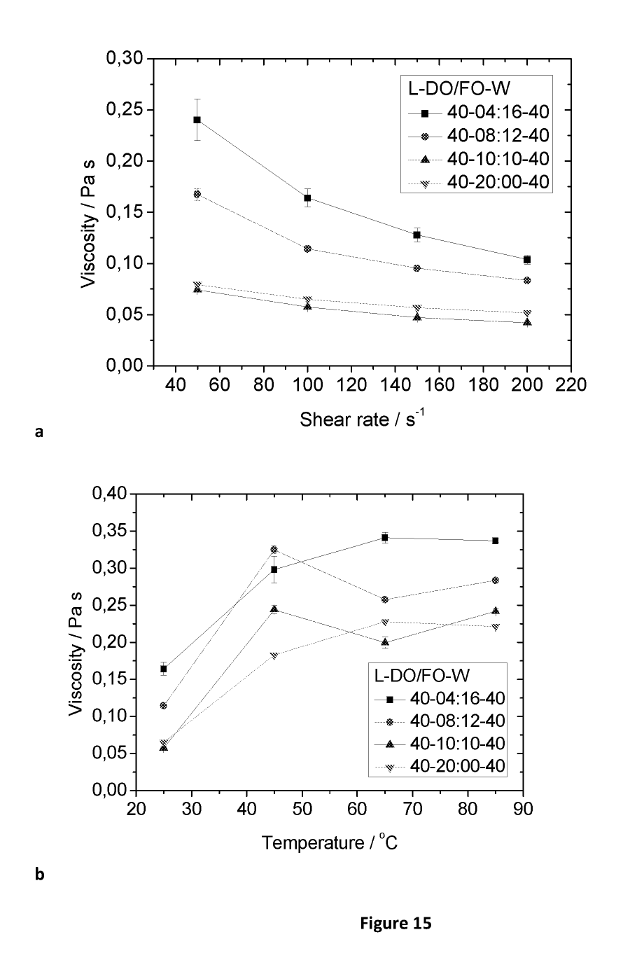

In FIG. 15, viscosity measurements are shown as a function of shear rate and temperature. The formulations all have 40% lignin, 40% water and 20% oil, with the ratio between diesel oil and heavy fuel oil varying between 4:16 and 10:10. For comparison, a formulation with no fuel oil (only diesel oil) is also shown. For the 40-20-40 formulation, replacing half of the diesel with fuel oil causes a small decrease in viscosity (of 17% at room temperature and shear rate 100 s⁻¹), as also described above. However, increasing the amount of heavy fuel oil causes a significant increase in viscosity.

In conclusion; for a variety of different LOW formulations, it is possible to replace a large fraction of diesel oil with heavy fuel oil and still have thin, pumpable liquids. However, there appears to be some limit to how much of diesel it is possible to replace.

### Example 15

### Filter Cake

Lignin filter cake has a water content of 40-50%. It is perfectly possible to prepare a thin, pumpable formulation of lignomulsion with a water content of less than 40% and a lignin content of more than 50%, when using grinded pellets. The fact that lignin filter cake is a hard and solid material gives the first indication that there may be some differences between filter cake and pellets/granulate.

Lignin filter cake is a hard material and is prepared for lignomulsion by cutting it into smaller pieces with the cutting function of the Kenwood machine. This results in a material with texture similar to earth, which can potentially be dried and milled to a fine powder.

Formulations were prepared using lignin filter cake from wheat straw treated at Inbicon in Kalundborg, Denmark, see Table 15-1. In some cases, lignin was dried at 50° C. to DM 82%.

| | | | | | | Lignin | Viscosity*/ |
|---|---|---|---|---|---|---|---|
| Sample name | L | O | W | AP10 | NaBenz. | DM/% | Pa s |
| 130517_004 | 20 | 0 | 80 | 0 | 0 | 59.07 ± 0.04 | 0.099 |
| 130530_001 | 25 | 0 | 75 | 0 | 0 | 59.07 ± 0.04 | 0.33 |
| 130522_001 | 30 | 0 | 70 | 0 | 0 | 59.07 ± 0.04 | 0.91 |
| 130522_002 | 30 | 10 | 60 | 0 | 0 | 59.07 ± 0.04 | 0.76 |
| 130523_001 | 30 | 10 | 60 | 0.5 | 0.5 | 59.07 ± 0.04 | 0.91 |
| 130529_001 | 35 | 20 | 45 | 0 | 0 | 59.07 ± 0.04 | NA |
| 130611_003 | 16 | 0 | 84 | 0 | 0 | Dried (82%) | 0.011 |
| 130613_001 | 20 | 0 | 80 | 0 | 0 | Dried (82%) | 0.022 |
| 130604_002 | 25 | 0 | 75 | 0 | 0 | Dried (82%) | 0.068 |
| 130610_001 | 25 | 10 | 65 | 0 | 0 | Dried (82%) | 0.057 |
| 130611_002 | 25 | 10 | 65 | 0.5 | 0.5 | Dried (82%) | 0.095 |
| 130612_002 | 30 | 10 | 60 | 0.5 | 0.5 | Dried (82%) | 0.18 |
| 130614_001 | 30 | 10 | 60 | 0 | 0 | Dried (82%) | 0.55 |
| 130621_002 | 25 | 20 | 55 | 0.0 | 0.0 | Dried (82%) | 0.47 |
| 130628_001 | 25 | 20 | 55 | 0.5 | 0.5 | Dried (82%) | 0.13 |
| 130628_002 | 29 | 20 | 51 | 0.5 | 0.5 | Dried (82%) | 0.41 |

*viscosity at 25° C. and shear rate 100 s⁻¹

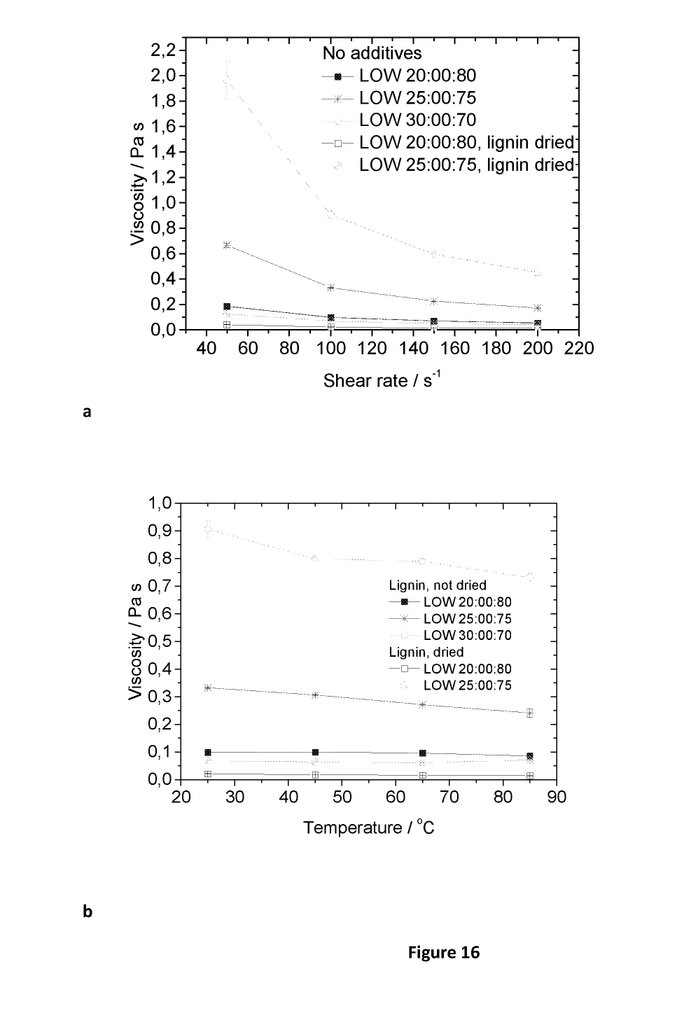

Working with not-dried lignin filter cake was difficult, as it resulted in highly viscous liquids, as is seen in FIG. 16. For example, at shear rate 100 s⁻¹, LOW 30-00-70 has a viscosity of 0.91 Pa s—for comparison a LOW formulation of 55-00-45 (ie almost twice as much lignin!) made from grinded pellets has a viscosity of 0.44 Pa s.

When, instead, lignin filter cake was pre-dried at 50° C. to a dry matter content of 82%, the result was a completely different material, very suitable for preparing low-viscosity emulsions—also shown in FIG. 16 (the difference between wet and dry lignin filtercake is further studied below). For example, the viscosity of LOW 25-00-75 formulation decreased from 0.33 Pa s when “not-dried” lignin filter cake was used-to 0.068 Pa s when instead using dried lignin filter cake, ie by almost 80%.

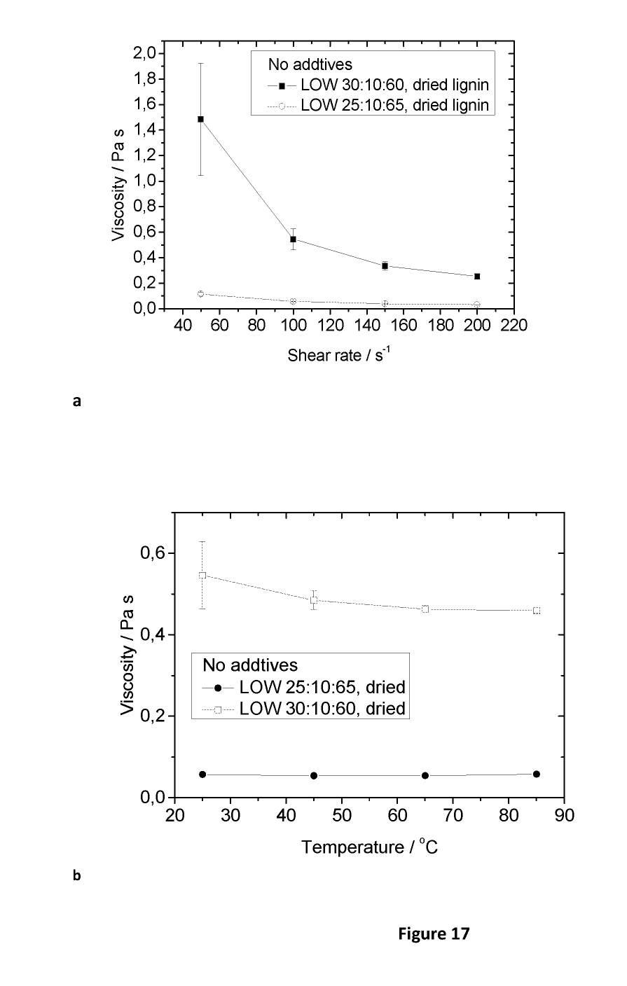

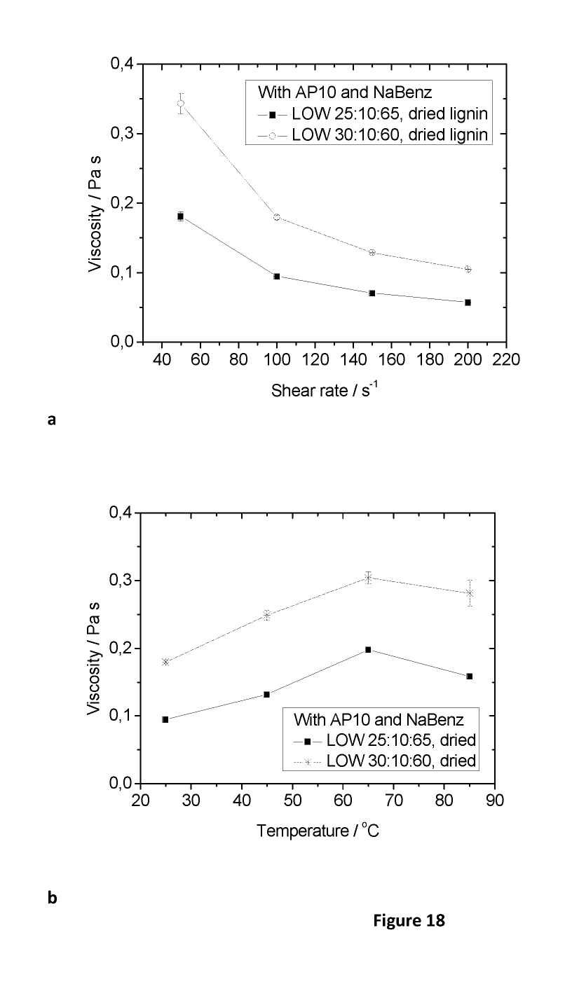

In FIGS. 17 and 18, the effect of lignin content is studied in the presence of 10% diesel oil when using dried filter cake, either with or without additives present. Again, an increase in lignin content results in an increase in viscosity. Also notice that in the absence of oil and/or additives, viscosity decreases with temperature (FIGS. 16 and 17), whereas in the presence of oil and additives, viscosities increases with temperature (FIG. 18). This is similar to what was observed for grinded pellets.

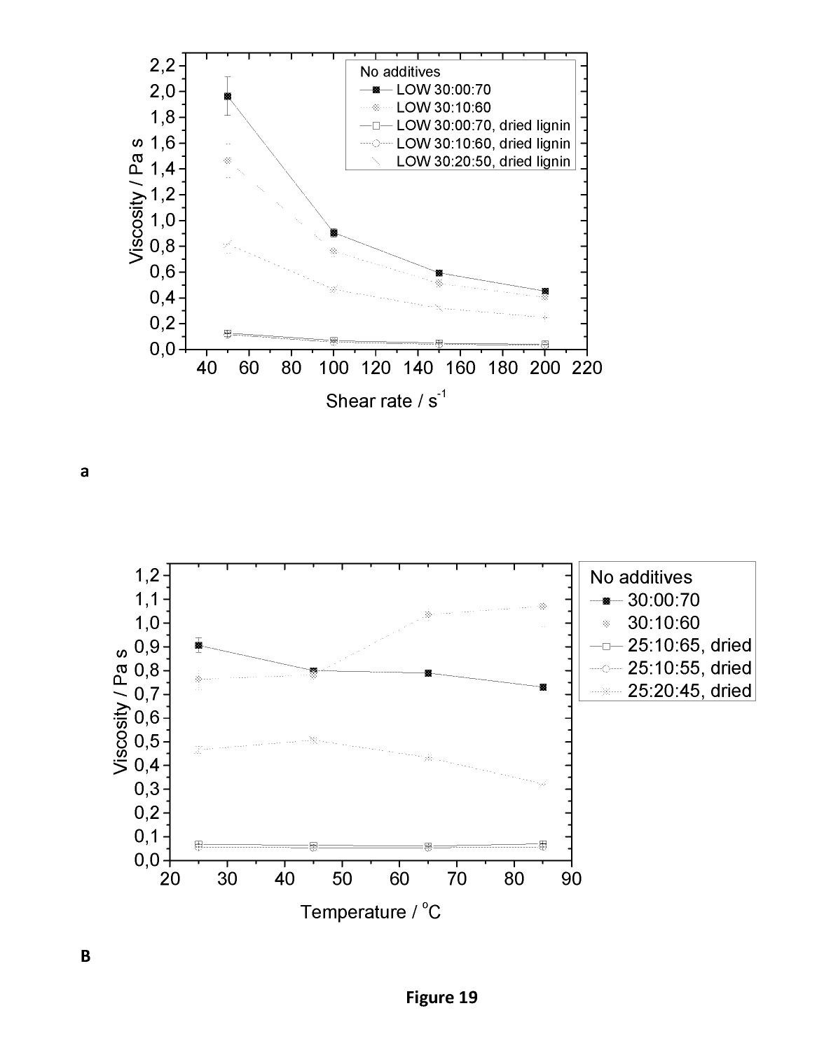

The effect of increasing the oil content was studied for both wet and dried (ie DM 82%) filtercake (FIG. 19), and an increase in oil content results in an increase in viscosity, similar to what was observed for grinded pellets.

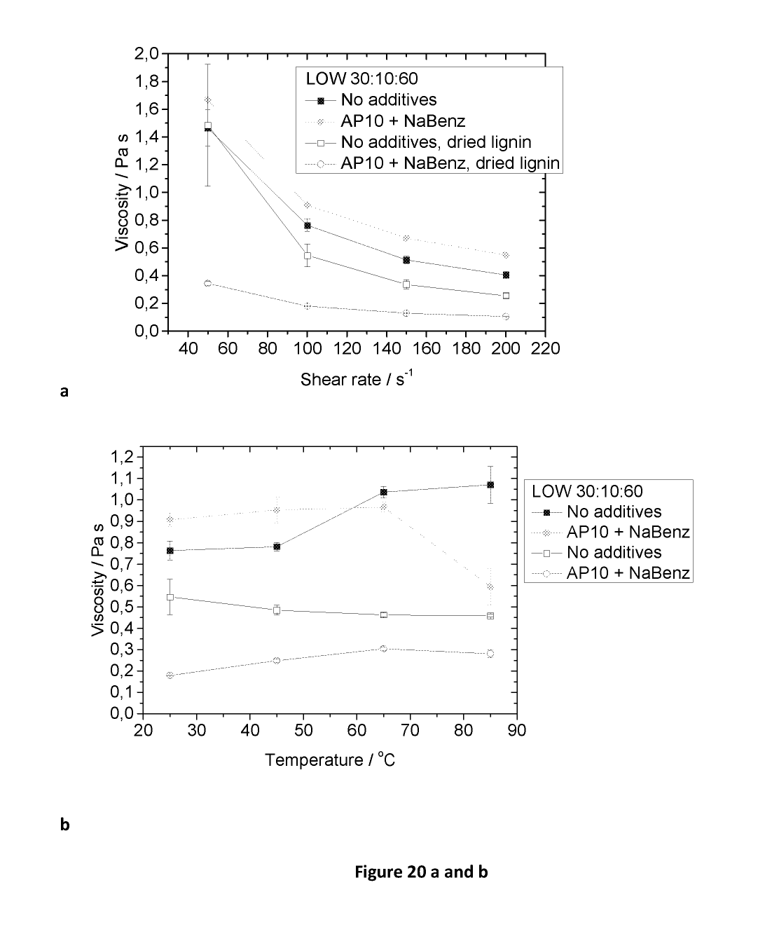

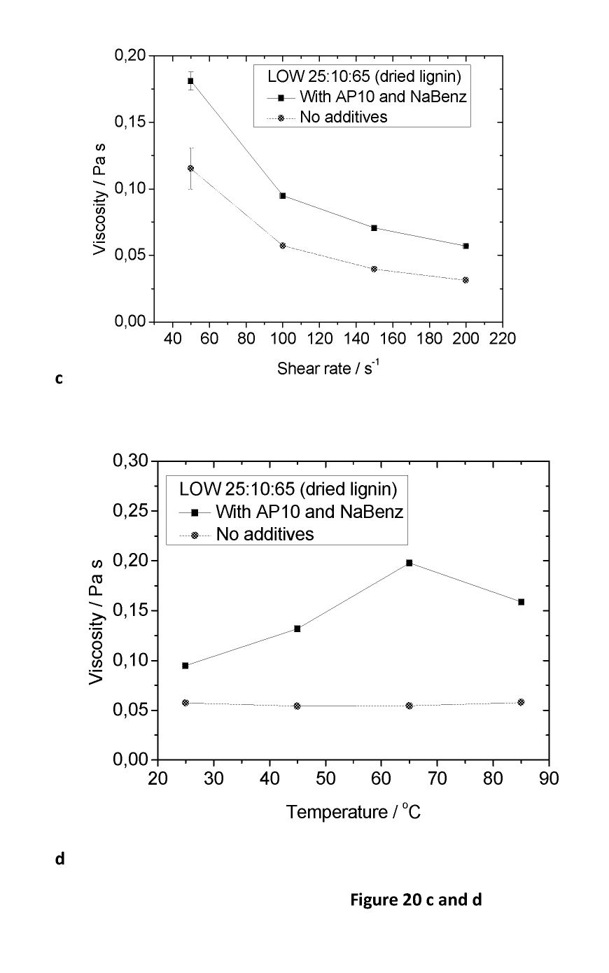

The effect of adding sodium benzoate and Lutensol AP10 was studied in two cases with different LOW composition (30:10:60 and 25:10:65, respectively); see FIG. 20. For the first composition (LOW 30:10:60; 20a and b), additives effectively decreased viscosity, whereas for the second composition (LOW 25:10:55; 20c and d) a slightly increase was observed when additives was added. Previously it was observed that for grinded pellets, an oil content of more than 10% was necessary to fully see the effects of additives.

Thus, using wet (and dried) filter cake is very similar to using grinded pellets in Lignomulsion, except that wet filter cake results in significantly higher viscosity formulations, and that lower lignin levels (and thereby lower fuel value) are needed to produce low viscosity liquids.

### Example 16

### Optimizing the Use of Filter Cake

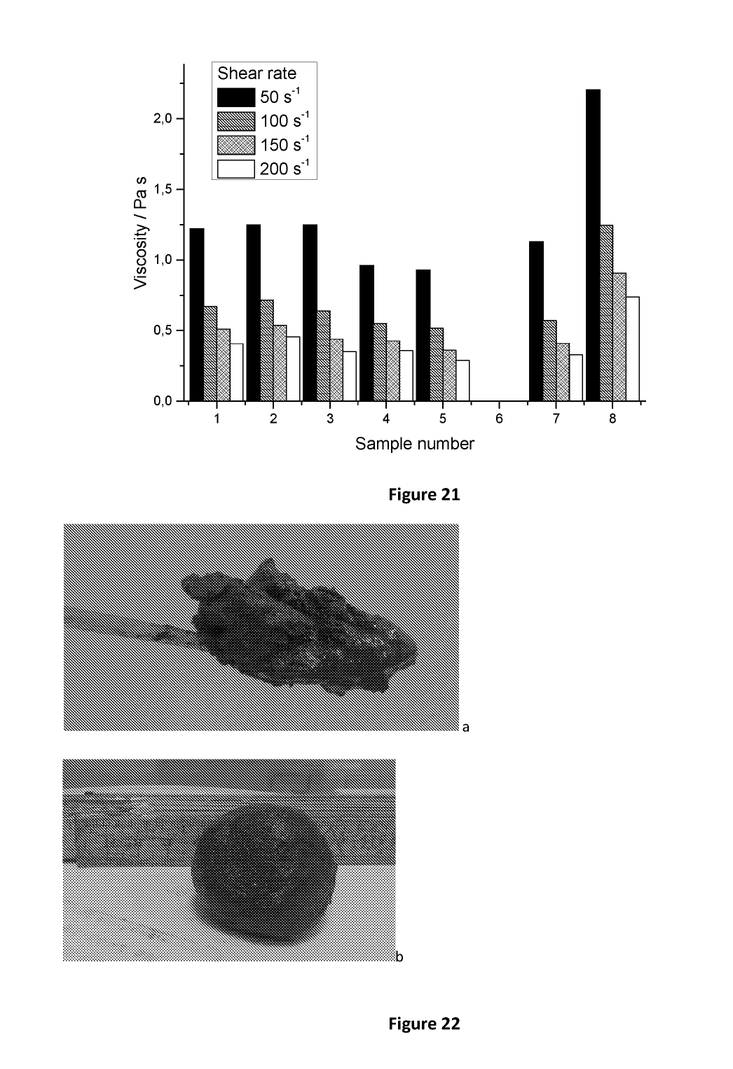

Since the use of wet filter cake (or decanter cake) are very relevant with regard to the overall economy of the process, there has been focus on how much “not-dried” lignin it is possible to suspend in an oil/water emulsion and still have low viscosity, and a different series of experiments were carried out, this time using a different stock of lignin filter cake (Filterkage fiber stillage IKA; 2013-09-02) with DM 54%. The details are given below in Table 16-1 and FIG. 21. First, a “standard” case was made with a LOW composition of 30:10:60 and with 5000 ppm of each of the additives Lutensol AP10 and sodium benzoate (additives 1A and 1B), mixed by using the ultraturrax at 10.000 rpm for 5 minutes.

The second sample had the same formulation, but the ultraturrax was only used for 1 minute. This did not change viscosity, and for the remaining samples, the ultraturrax was used for 5 minutes.

In the third sample, the concentrations of the additives were increased 5 fold, but this did not lower viscosity either.

In the fourth sample, different additives; RD193295 and Lutensol TO15 (called 2A and 2B) were used. From the results in Example 11, this was expected to lower viscosity. This was also the case.

In the fifth sample, concentrations of additives 2A and 2B were increased 5 fold, but with no decrease in viscosity compared to the fourth sample.

In the sixth sample, oil content was increased to 30% (by lowering water content), but this resulted in a fluid too viscous for measuring viscosity.

In the seventh sample, half of the (diesel) oil content was replaced by heavy fuel oil, which did not affect viscosity.

Finally, in the 8^{th} eights sample a different lignin stock with a lower dry matter (DM 36.5%) was used, resulting in a much higher viscosity. This was an example of decanter cake from Inbicon.

TABLE 16-1
List of formulations for optimisation the use of wet lignin filter cake in Lignomulsion

| | Sample | L | O | W | Add. A | Add. B | Viscosity* | Comment |
|---|---|---|---|---|---|---|---|---|
| 1 | 140326_001 | 30 | 10 | 59 | 0.5 | 0.5 | 0.67 | Ultraturrax 5 min additive 1A. 1B |
| 2 | 140326_002 | 30 | 10 | 60 | 0.5 | 0.5 | 0.72 | Ultraturrax 1 min; additive 1A. 1B |
| 3 | 140326_003 | 29 | 10 | 59 | 1.2 | 1.2 | 0.64 | Extra additive 1A. 1B |
| 4 | 140326_004 | 29 | 10 | 60 | 0.5 | 0.5 | 0.55 | Additive 2A. 2B |
| 5 | 140328_001 | 29 | 10 | 58 | 1.3 | 1.2 | 0.52 | Extra additive 2A. 2B |
| 6 | 140328_002 | 29 | 29 | 40 | 1.2 | 1.2 | NA | Different LOW; additive 2A. 2B |
| 7 | 140328_003 | 29 | 10 | 58 | 1.2 | 1.2 | 0.57 | 1:1 diesel-fuel oil mixture. |
| | | | | | | | | additive 2A. 2B |
| 8 | 140411_002 | 29 | 10 | 59 | 1.2 | 1.2 | 1.25 | Different lignin; additive 2A. 2B |

*viscosity at 25° C. and shear rate 100 s⁻¹

In conclusion; wet filter cake is quite difficult to work with, but a formulation of LOW 30-10-60 seems to result in a thick, pourable liquid. Drying filter cake completely changes the material, making it possible to add more lignin and still obtain a thin fluid. The effect of drying is studied below.

### Example 17

Comparison with Kraft Lignin

Lignomulsion was prepared from Indulin (supplied by MeadwestVaco), according to Table 17-1.1

TABLE 17-1
Formulation of Lignomulsion prepared with Indulin (DM 98%), LOW 40-15-45

| | | Lignin | Oil | Water | AP8 | AP10 | Glucose | TO15 | RD193295 | Stability |
|---|---|---|---|---|---|---|---|---|---|---|
| Sample ID | Lignin type | % | % | % | % | % | % | % | % | index* |
| 140528_001 | Indulin | 40 | 15 | 45 | 0.2 | 0.2 | 0 | 0 | 0 | 29.4 |
| 140604_001 | Indulin | 40 | 15 | 45 | 0 | 0 | 0 | 1 | 1 | 7.9 |
| 140603_001 | Indulin | 40 | 15 | 45 | 0.2 | 0.2 | 1 | 0 | 0 | 75.4 |
| 140617_001 | Indulin | 40 | 15 | 45 | 0.2 | 0.2 | 5 | 0 | 0 | 75.0 |
| 140616_001 | Indulin | 40 | 15 | 45 | 0.2 | 0.2 | 10 | 0 | 0 | 86.3 |
| 140617_002 | Grinded | 40 | 15 | 45 | 0.2 | 0.2 | 0 | 0 | 0 | <3000 |
| | pellets | | | | | | | | | |

*Defined as the number of minutes at shear rate 100 s⁻¹ necessary for the viscosity to reach 2.1 Pa s.

Sample 140528_001 is a reproduction of an example from Patent WO96/10067 (Title: Lignin water oil slurry fuel); see the extract and link below:

Example 4
160 g of Terigtol ™ NP-9 together with 35.2 kg of water and 12 kg of
fuel oil No. 2 were homogenised in a tank to form an oil; -in-water
emulsion. 32.65 kg of Indulin AT ™ lignin (sold by Westvaco.)
containing 2% by weight moisture was added gradually (in portions) to
the oil-in-water emulsion and mixed under high shear conditions at a
temperature between 18 and 25° C. The resultant stable slurry contained
40% lignin; 15% fuel oil no. 2; 44.8% water; and 0.2% (i.e. 2,000 ppm)
surfactant. This produced an 80 kg sample for testing in a lime kiln.

Note that the “Terigtol NP” series are Nonylphenol ethoxylate of various molecular size, similar to the “Lutensol AP” series. Lutensol AP8 and AP10 were therefore mixed to obtain an additive with the same hydrophilic-lipophilic balance.

In samples 140603_001, 140616_001 and 140617_001, 1-10% glucose was added in an attempt to make Indulin more comparable to Inbicon lignin, which has a ^{˜}10% carbohydrate content composed mainly of glucan. Indulin has a glucan content of 0.11% and a xylan content of 0.37%. In sample 140604_001, different additives were used. These are among those tested in the above examples and found to be among the most optimal for lowering viscosity. Finally, a LOW formulation with grinded lignin pellets from Inbicon was prepared for comparison.

Oil, water and additive was mixed and homogenized with an ultraturrax to prepare an emulsion to which Indulin was added gradually in portions of 50-100 g. This was homogenized with an ultraturrax for 10 minutes at 20,000 rpm.

Immediately after homogenizing sample 140528_001 (the reproduction of the patent example), it could be poured into another container, although it was quite viscous. However, 10 minutes after, a spoonful of matter was scooped out of the container. As seen in FIG. 22a, the liquid character of the slurry is much reduced.

FIG. 22b shows how a handful of the slurry could be formed to a small ball (diameter of 5 cm). The ball was left for several hours, and during this time, no change in physical appearance was observed. There is every reason to assume that the ball could have kept its shape for any duration.

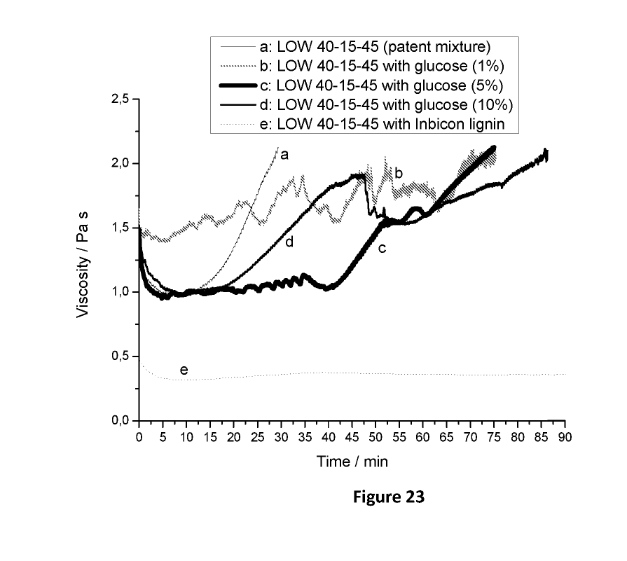

Viscosity was measured of the slurries shown in Table 17-1; 40 ml slurry was poured into the cup of the Viscotester VT550 to measure viscosity at shear rate 100 s⁻¹. The results are shown in FIG. 23.

Initially, viscosity of the formulations prepared with Indulin decreases over time until reaching a minimum. Then viscosity increases until the slurry is so viscous (viscosity=2.1 Pa s) that the instrument stopped. This duration (in minutes) is defined as the “Stability index”.

Analysis of Sample 140528_001 stopped after 30 minutes. Sample 140604_001 showed the “optimal” additives were not very efficient for lowering viscosity of Indulin Lignomulsion, and a viscosity of 2.1 Pa s beyond the capacity of the instrument was reached in less than 10 minutes. Using glucose as an additive was slightly more efficient. Viscosity decreased slowly, and the of viscosity 2.1 Pa s, at which the instrument could no longer measure was reached, after more than 75 minutes.

However, in all cases the formulation was far from liquid and very difficult to pour or pump. As Lignomulsion prepared from Kraft lignin went from liquid and pourable to highly viscous and semi-solid within minutes, Kraft lignin is not suitable for preparing a lignin based lignin fuel.

### Example 18

### Effects of Drying

The difference between lignin filter cake and pellets, is that the pellets have been dried (to 95% DM), pelletized and grinded. Observations indicate that drying could be conceived as an important step, altering the physical and chemical properties of lignin. Therefore, different samples of lignin filter cake and decanter cake have been dried under different conditions, and their properties in Lignomulsion have been assessed.

### Temperature Effect

Lignin filtercake was dried to DM>95% at four different temperatures (30-100° C.) according to Table 18-1. Drying at low temperature took several days.

Formulations were prepared with the composition LOW 30-20-50 and the addition of 5000 ppm sodium benzoate and 5000 ppm Lutensol AP10, see Table 18-1. Viscosity was measured at the following shear rates: 50, 100, 150 and 200 s⁻¹, at the following temperatures: 25, 45, 65 and 85° C.

TABLE 18-1

| ID | L-O-W | Drying T/° C. | DM/% | Viscosity*/Pa s |
|---|---|---|---|---|
| 130705_001 | 30-20-50 | 30 | 97.4 ± 0.4 | 0.232 |
| 130705_002 | 30-20-50 | 50 | 99.6 ± 0.2 | 0.234 |
| 130704_001 | 30-20-50 | 80 | 100.5 ± 0.2 | 0.228 |
| 130704_002 | 30-20-50 | 100 | 101.3 ± 0.1 | 0.141 |

*Measured at room temperature and shear rate 100 s⁻¹.

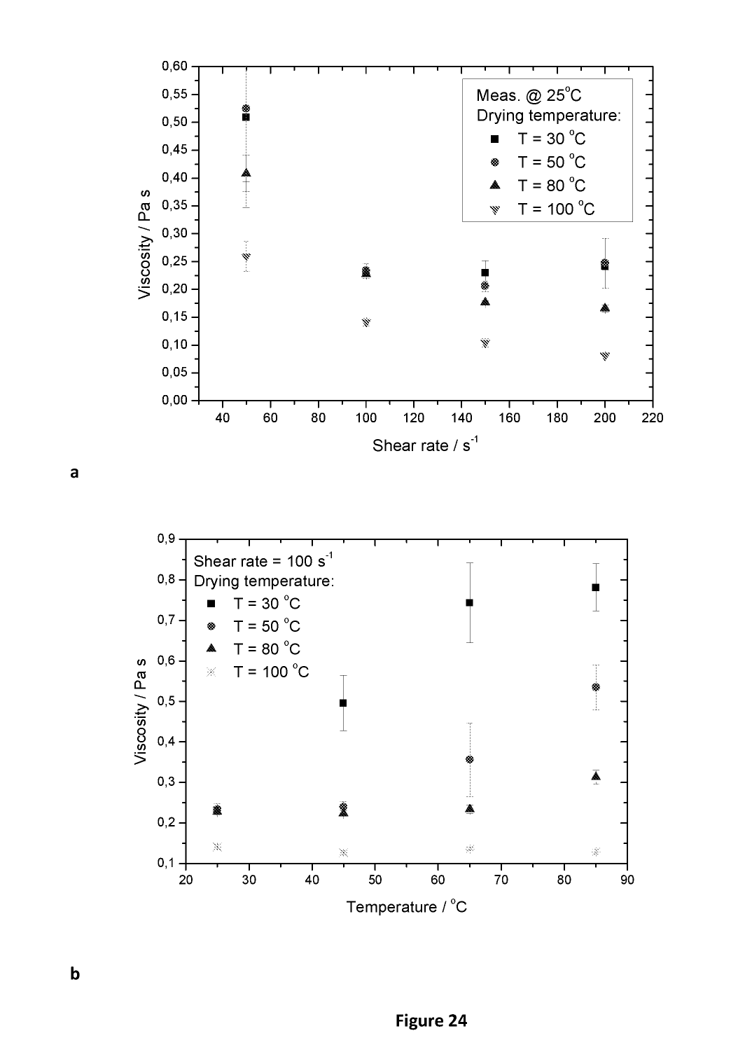

FIG. 24 shows viscosity as a function of shear rate (a) and of temperature (b). At room temperature, here appears to be a connection between viscosity and drying temperature, as the emulsions prepared with lignin dried at the lowest temperatures (30-50° C.) are more viscous than emulsions prepared from lignin dried at the highest temperatures (80-100° C.).

This is also correlated to the temperature dependency of the viscosity measurements: Viscosity of LOW formulations from filter cake dried at 80-100° C. does not change as a function of temperature. This indicates that the transformations in the emulsions causing viscosity to change is some sort of heat initiated change of lignin.

### DM Effect

Lignin filter cake was dried in various degrees (DM=59-100%) at 50° C., according to table 18-2.

Formulations were prepared with the composition LOW 30-20-50 with the addition of 5000 ppm sodium benzoate and 5000 ppm Lutensol AP10, see table 18-2. Viscosity was measured at the following shear rates: 50, 100, 150 and 200 s⁻¹, at the following temperatures: 25, 45, 65 and 85° C.

TABLE 18-2
Formulations

| ID | L-O-W | Drying T/° C. | DM/% | Viscosity*/Pa s |
|---|---|---|---|---|
| 130710_002 | 30-20-50 | 50 | 59.07 ± 0.04 | 0.327 |
| 130709_001 | 30-20-50 | 50 | 65.4 ± 0.2 | 0.307 |
| 130710_001 | 30-20-50 | 50 | 73 ± 1 | 0.271 |
| 130711_001 | 30-20-50 | 50 | 82 ± 2 | 0.139 |
| 130709_002 | 30-20-50 | 50 | 99.6 ± 0.2 | 0.238 |

*Measured at room temperature and shear rate 100 s⁻¹.

The object of these measurements was to find out if the change in properties between wet and dry material, as noted above, appears gradually or suddenly. Lignin was therefore dried for different durations resulting in different dry matter contents, all at the same temperature. It is not possible to completely characterize the drying conditions through temperature and duration, as it also depends on for example the exposed surface properties of the wet material (eg particle size and the shape of the container of the wet lignin). Some uncertainty is therefore expected.

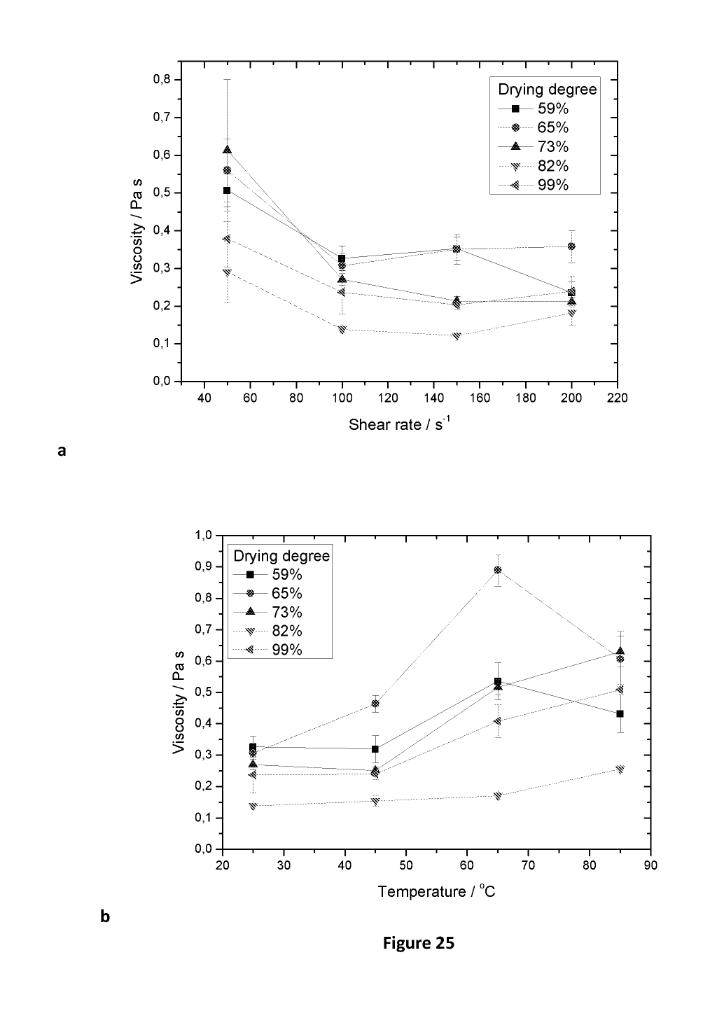

This is also reflected in FIG. 25, showing viscosity as a function of shear rate (a) and of temperature (b). No clear connection between dry matter content and viscosity can be observed, although the three wettest materials (DM 59%, 65% and 73%) result in more viscous emulsions than the two driest materials (82% and 99%). However, this effect is not significant enough to draw any conclusions.

### Example 19

### Heated Lignin Experiment

As lignin dried at high temperatures resulted in emulsions with low viscosities, a study was made to determine if preparing lignomulsion at high temperature resulted in different properties. 85° C. was selected as the maximum temperature that could be safely handled in the lab.

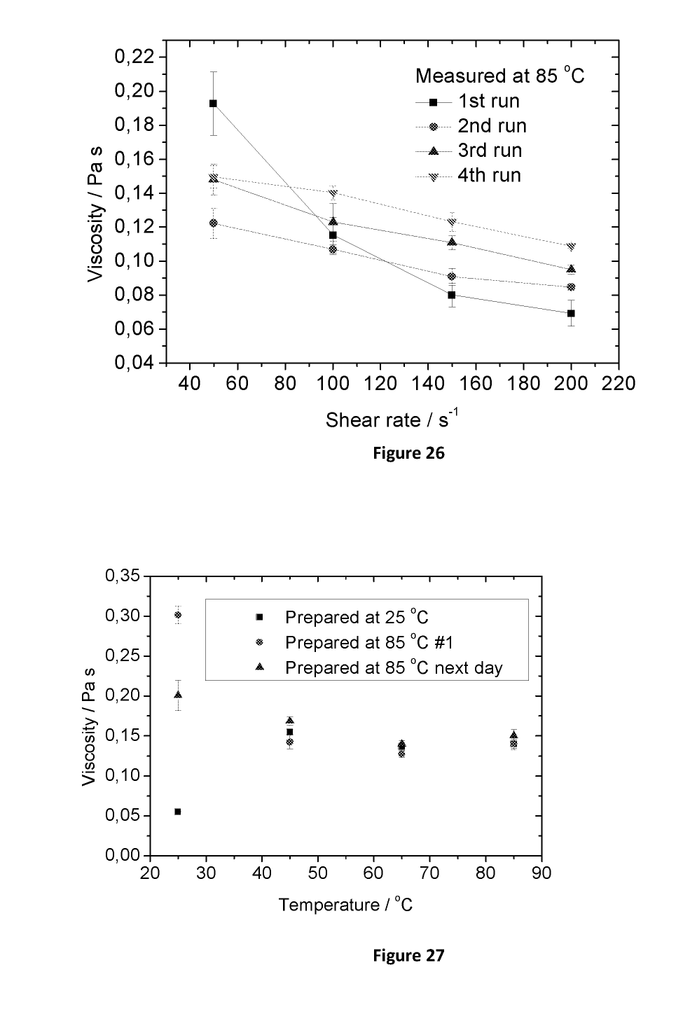

A formulation with LOW 40-20-40 (with grinded pellets as the lignin source) and 5000 ppm sodium benzoate and 5000 ppm Lutensol AP10. All ingredients were heated to 85° C. and mixed (using the ultraturrax for 5 minutes and 10.000 rpm) while in a 85° C. water bath. Viscosity was measured, first at 85° C. at shear rates 50, 100, 150 and 200 s⁻¹; this was repeated a total of four times (see FIG. 26). Then the formulation was cooled to 25° C. and viscosity was re-measured. This measurement was repeated at 25° C. the next day.

These sets of measurements were compared to Lignomulsion with the same formulation (40-20-40 and 5000 ppm Sodium benzoate and Lutensol AP10), see FIG. 27. No variation in viscosity is observed above room temperature, but the viscosity at 25° C. is higher in the Lignomulsion prepared at 85° C. It seemed to decrease after one day of storage.

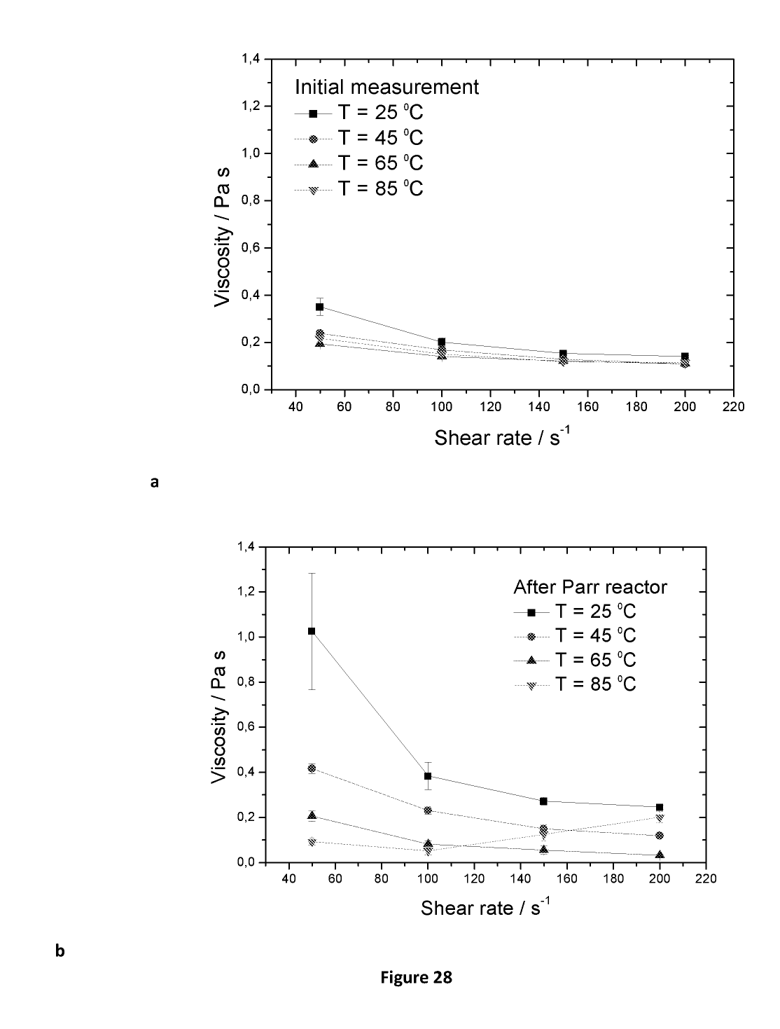

Finally, a LOW 30-00-70 formulation using lignin filter cake was treated in the Parr reactor at 10 min at 120° C. and then cooled to room temperature. As drying lignin at elevated temperature was found to reduce viscosity, it was tested if it was the drying or the high temperature that caused the change. Viscosity was therefore measured before and after this treatment, see FIG. 28. Viscosity decreases with increasing shear rate and increasing temperature, which is also expected for a formulation with no oils and no additives. At low temperature (25-45° C.), the treatment of the Parr reactor caused viscosity to increase significantly, whereas at high temperature (65-85° C.) viscosity decreased slightly. In conclusion, from the Parr reactor experiments and the “85° C. preparation” experiments there is no advantage to exposing lignin or lignomulsion to high temperatures under wet conditions.

### Example 20

### Lignin, Dried Types

As drying conditions and dry matter content was found to be highly important for Lignomulsion prepared from lignin filter cake, we tried to find out if the same effects applied to pellets. Lignin pellets from the same campaign (spring, 2012) and different dry matter contents (84%, 89% and 95%) were tested, as well as pellets and granulate with the same dry matter contents (^{˜}95%). In the following, these materials are referred to as “84% pellets”, “89% pellets”, “95% pellets” and “95% granulate”.

Dry matter content was re-analyzed, and the results are given below in Table 20-1.

TABLE 20-1
Properties of raw material

| | Type | DM(1)/% | DM(2)/% |
|---|---|---|---|
| A | Pellets | 84 | 91.84 ± 0.04 |
| B | Pellets | 89 | 94.22 ± 0.03 |
| C | Pellets | 95 | 96.08 ± 0.05 |
| D | Granulate | 95 | 93.92 ± 0.06 |

DM(1) is the dry matter content determined first. DM(2) lists the results after re-analysis. The pellets were drier than expected, whereas the granulate had taken up a little water. The discrepancies could be due to water uptake/evaporation during storage or grinding, and it gives a first indication that pellets and granulate are two fundamentally different materials.

“Stock emulsions” of grinded lignin pellets/granulate and water (65:35 by mass) were prepared and used for preparing emulsions of three different formulation, see Table 20-2. 5000 ppm sodium benzoate and 5000 ppm Lutensol AP10 were added.

TABLE 20-2
Formulations with different lignin types

| | L | O | W | AP10 | NaBenz | | Viscosity* |
|---|---|---|---|---|---|---|---|
| Sample ID | % | % | % | % | % | Lignin type | Pa s |
| 131202_001 | 40 | 20 | 40 | 0.5 | 0.5 | Pellets, 84% | 0.174 |
| 131202_002 | 40 | 30 | 30 | 0.5 | 0.5 | Pellets, 84% | 0.111 |
| 131202_003 | 30 | 30 | 40 | 0.5 | 0.5 | Pellets, 84% | 0.090 |
| 131203_001 | 40 | 20 | 40 | 0.5 | 0.5 | Pellets, 89% | 0.097 |
| 131203_002 | 40 | 30 | 30 | 0.5 | 0.5 | Pellets, 89% | 0.096 |
| 130312_001 | 40 | 20 | 40 | 0.5 | 0.5 | Pellets, 95% | 0.065 |
| 131216_001 | 40 | 30 | 30 | 0.5 | 0.5 | Pellets, 95% | 0.849 |
| 130308_001 | 30 | 30 | 40 | 0.5 | 0.5 | Pellets, 95% | 0.047 |
| 131127_001 | 40 | 20 | 40 | 0.5 | 0.5 | Granulate, 95% | 0.059 |
| 131127_002 | 40 | 30 | 30 | 0.5 | 0.5 | Granulate, 95% | 0.123 |
| 131127_003 | 30 | 30 | 40 | 0.5 | 0.5 | Granulate, 95% | 0.023 |

Viscosity was measured with a Haake ViscoTester VT550 at shear rates 50, 100, 150 and 200 s⁻¹ and the following temperatures: 298, 318, 338 and 358 K (25-85° C.).

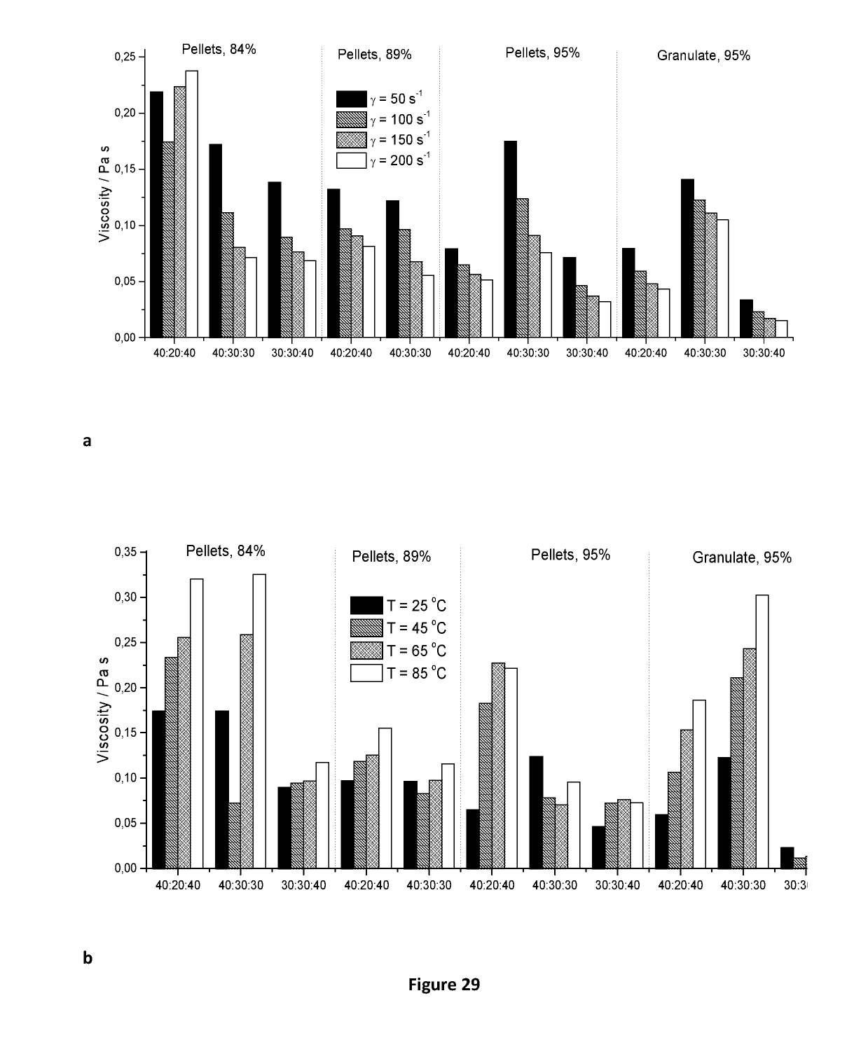

First, the four emulsions with LOW composition 40-20-40 (see also Table 20-2) were compared, see FIG. 29a-b: Overall, there is not much difference between pellets and granulate. At room temperature, viscosity of emulsions from granulate and pellets dried to RH 95% are very similar, at all four measured shear rates and all four temperatures. The pellets dried to RH 84 and 89% result in slightly more viscous formulations at room temperature. It has previously been shown for filter cake that drying prior to making Lignomulsion results in a significant reduction in viscosity. It is therefore not surprising that the level of dryness is correlated to viscosity. When temperature is increased, in all cases, viscosity increases with increasing temperature (probably due to additive facilitated lignin-water interaction, resulting in water uptake), and the temperature gradient is slightly greater for 95% pellets than for granulate, whereas the temperature gradient is minimal for the 84% pellets. There is no clear relation between viscosity and original lignin dry matter content at temperatures above room temperature.

Then, these results were compared to the four formulations with LOW composition 40-30-30 to evaluate the effect of replacing 10% of water with 10% oil. This has a different effect on viscosity for the four different lignin types: For the pellet types with the lowest DM (ie 84% and 89%) at room temperature, the extra oil causes viscosity to decrease, but for the driest material (95% pellets and granulate) it causes increases viscosity. At higher temperatures, increasing oil content causes viscosity to decrease in the formulations prepared from the three pellet types, but viscosity increases in formulations prepared from granulate.

Finally, the four formulations with LOW composition 30-30-40 were evaluated. Please note, that there is no data for LOW 30-30-40 containing lignin dried to RH 89%. By comparing this set of measurements with the LOW 40-20-40 formulations, we evaluate the effect of replacing 10% of lignin with 10% oil at a constant water content. The conclusion is that when lignin content goes down, so does viscosity. This is seen for all lignin types, temperatures and shear rates.

By comparing LOW 30-30-40 with LOW 40-30-30, we can evaluate the effect of replacing 10% of lignin with 10% water at a constant oil content. Again, it is seen that when lignin content goes down, so does viscosity (for all lignin types, temperatures and shear rates).

Evaluating viscosity of the four formulations with the same LOW composition, it is seen that at room temperature the different lignin materials result in different viscosities, with the 84% pellets resulting in the highest viscosity, but also with a big difference between the 95% pellets and granulate.

However, it is noted that on an absolute scale, the viscosities of the studied emulsions appear quite low.

In terms of viscosity, it is apparently an advantage that the lignin is as dry as possible, since the 95% material generally results in emulsions of viscosities lower than those prepared from the 84% or 89% material.

2) Using granulate in Lignomulsion result in low viscosity, comparable to formulations prepared from pellets. Increasing the amount of oil in Lignomulsion may have different effects on lignomulsion viscosity.

### Example 21

### Sugar Content

It was postulated that the presence of sugars and other residues from the Inbicon 2G process has beneficial properties for Lignomulsion, especially with regard to viscosity and stability. This should be seen in contrast to for example Kraft lignin.

In an initial, three different types of lignin were studied: One was Indulin AT from Westvaco. The other two were from ISK (13-R4-23-8, 8/5 11:20 Fiber mash—designated “Inbicon lignin”)—they had undergone same pretreatment, hydrolysis and fermentation (A), but one of them was then given an extra hydrolysis (with approximately 300 g enzyme per kg sugar) and fermentation (B). The resulting material was dried at 50° C. to a dry matter content (DM) of >99.5%. The composition was then analysed (according to the NREL procedure) with the following results:

TABLE 21-1
Chemical compostion of 13-R4-23-8 with single and double hydrolysis

| | Glucan | Xylan | Arabinose | Klason lignin | Ash | Total |
|---|---|---|---|---|---|---|
| 13-R4-23-8 (A) | 6.89% | 2.94% | <0.5% | 61.8% | 10.5% | 82.10% |
| 13-R4-23-8 (B) | 2.52% | 2.77% | <0.5% | 72.7% | 9.9% | 87.88% |
| ISK 2012-03-28 (C) | 27.4% | 9.9% | 0.8% | 50.9 | 6.5 | 95.5% |

Also included in the table above is a third type of filtercake which for some reason has a very high content of sugar (C). To increase the range of sugar concentrations in the prepared formulations, the different lignin types were mixed, according to Table 21-2:

| | | L (%) | L (%) | | | | |
|---|---|---|---|---|---|---|---|
| | L (%) | l × | 2 × | | | NaBenz | AP10 |
| ID | Indulin | hydro. | hydro. | O (%) | W (%) | Ppm | Ppm |
| 130702_001 | 30 | 0 | 0 | 10 | 60 | 5000 | 5000 |
| 130702_002 | 15 | 15 | 0 | 10 | 60 | 5000 | 5000 |
| 130702_003 | 15 | 0 | 15 | 10 | 60 | 5000 | 5000 |
| 130702_004 | 7.5 | 22.5 | 0 | 10 | 60 | 5000 | 5000 |
| 130702_005 | 7.5 | 0 | 22.5 | 10 | 60 | 5000 | 5000 |
| 130701_002 | 0 | 30 | 0 | 10 | 60 | 5000 | 5000 |
| 130701_003 | 0 | 0 | 30 | 10 | 60 | 5000 | 5000 |

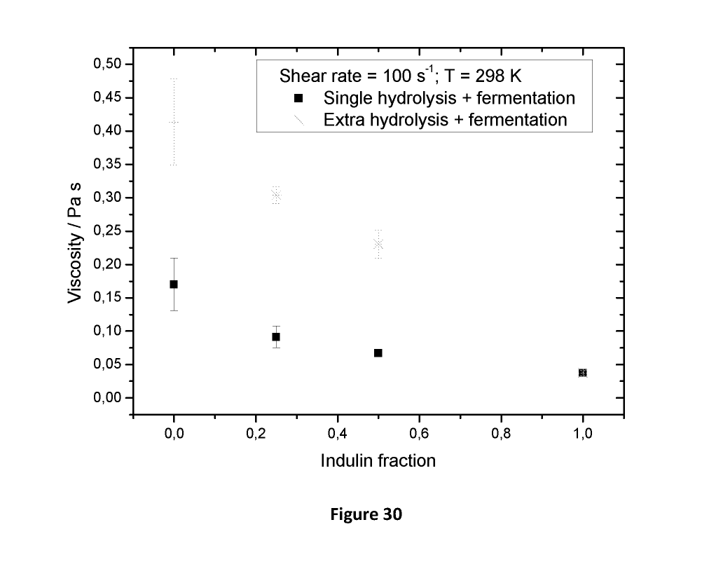

FIG. 30 shows that viscosity of a LOW 30-20-50 emulsion where the lignin content is solely made up of Indulin is much lower than emulsions were the lignin content is either partially or completely composed of Inbicon lignin. This definitely demonstrates that there is some difference between Inbicon lignin and Kraft lignin.

Some additional formulations were prepared, see table 21-2:

| | Lignin (%) | | ISK | | | | | | | |
|---|---|---|---|---|---|---|---|---|---|---|
| | 13-R4-23-8 | | 2012-03-28 | | | Contribution to dry matter | | | | Viscos [symbol] |
| Name | A | B | | Oil | Water | Klason lignin | Glucan | Xylan | Glucan + xylan | s⁻¹ [symbol] |
| 130701_002 | 30 | 0 | 0 | 10 | 60 | 18.54 | 2.07 | 0.88 | 2.95 | 0.1 [symbol] |
| 130701_003 | 0 | 30 | 0 | 10 | 60 | 21.81 | 0.76 | 0.83 | 1.59 | 0.4 [symbol] |
| 130719_001 | 15 | 15 | 0 | 10 | 60 | 20.18 | 1.41 | 0.86 | 2.27 | 0.2 [symbol] |
| 130719_002 | 7.5 | 22.5 | 0 | 10 | 60 | 20.99 | 1.08 | 0.84 | 1.93 | 0.2 [symbol] |
| 130722_002 | 0 | 22.5 | 7.5 | 10 | 60 | 20.18 | 2.62 | 1.37 | 3.99 | 0.2 [symbol] |
| 130723_002 | 22.5 | 0 | 7.5 | 10 | 60 | 17.72 | 3.61 | 1.40 | 5.01 | 0.2 [symbol] |
| 130723_003 | 15 | 0 | 15 | 10 | 60 | 16.91 | 5.14 | 1.93 | 7.07 | 0.1 [symbol] |
| 130724_001 | 0 | 7.5 | 22.5 | 10 | 60 | 16.91 | 6.35 | 2.44 | 8.79 | 0.2 [symbol] |
| 130724_002 | 7.5 | 0 | 22.5 | 10 | 60 | 16.09 | 6.68 | 2.45 | 9.13 | 0.1 [symbol] |
| 130724_003 | 0 | 15 | 15 | 10 | 60 | 18.54 | 4.49 | 1.90 | 6.39 | 0.3 [symbol] |
| 130723_001 | 0 | 0 | 30 | 10 | 60 | 15.27 | 8.22 | 2.97 | 11.19 | 0.1 [symbol] |

[symbol]

indicates data missing or illegible when filed

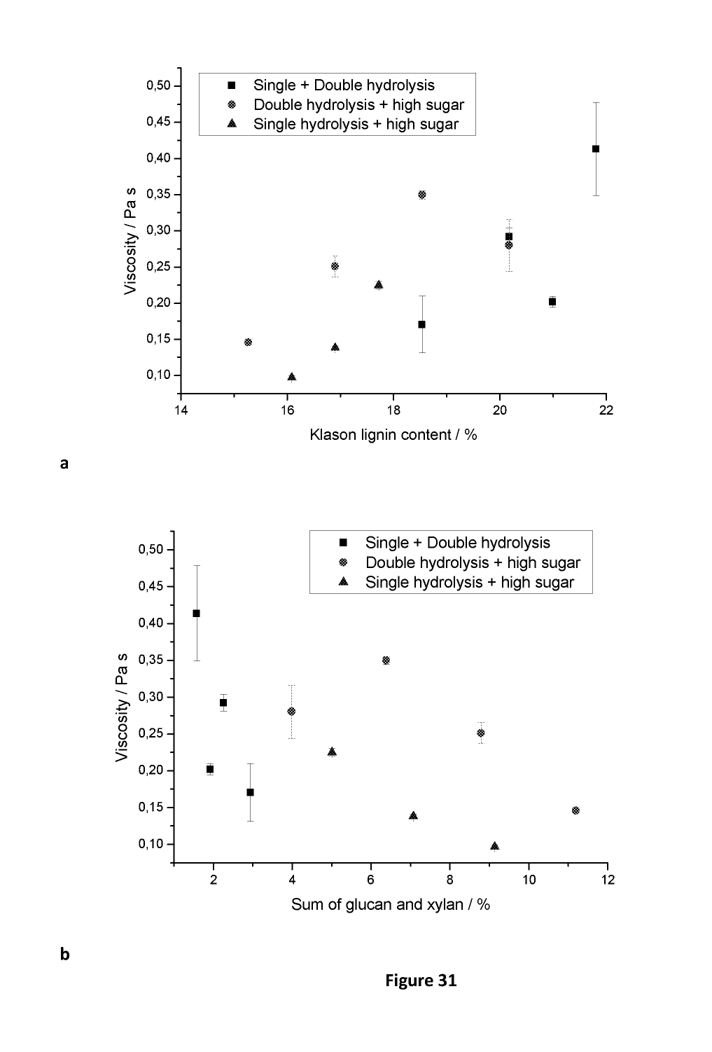

Since the lignin and sugar content is different in each sample, viscosity is displayed in FIG. 31 as a function of content of klason lignin and sugar (determined as the sum of xylan and glucan). Viscosity is measured at room temperature and shear rate 100 s⁻¹.

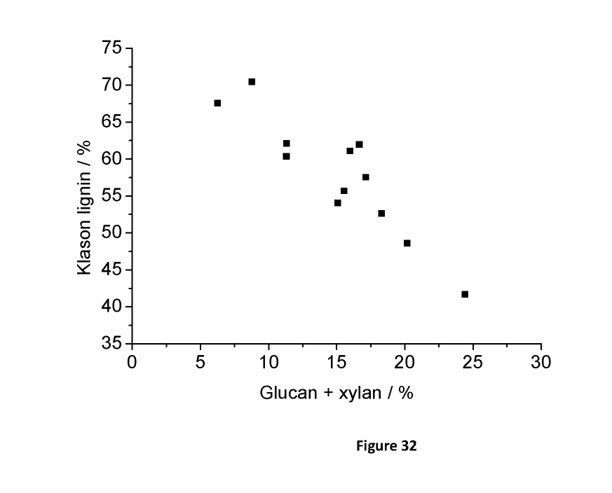

As a rule, viscosity increases with klason lignin content and decreases with sugar content. However, since the trends in FIG. 31 are mainly observed for the formulations containing the same lignin samples, a new series of lignin samples were prepared from three different materials pretreated at three different severities; these either underwent a “normal” hydrolysis (60 g/kg glucan) or hydrolysis with a five times extra enzyme dosis, and in some cases fermented. The effect of pretreatment is clearly seen from the measured xylan, but not in the measured glucan. On the other hand; the effect of different enzyme dosages and fermentation is mainly reflected in the measured glucan content. Both of these effects are reflected in the lignin content, which is correlated to the combined sugar content; see FIG. 32.

The samples were dried at 50° C. and milled to ensure a homogeneous particle size distribution below 2 mm. This material was used to make Lignomulsion with LOW formulations 30-20-50 and 40-10-50, both with 5000 ppm Lutensol AP10 and sodium benzoate, respectively. In all cases, viscosity decreased with shear rate and only the measurements at shear rate 100 s⁻¹ will be shown in the following (see also Table 21-3).

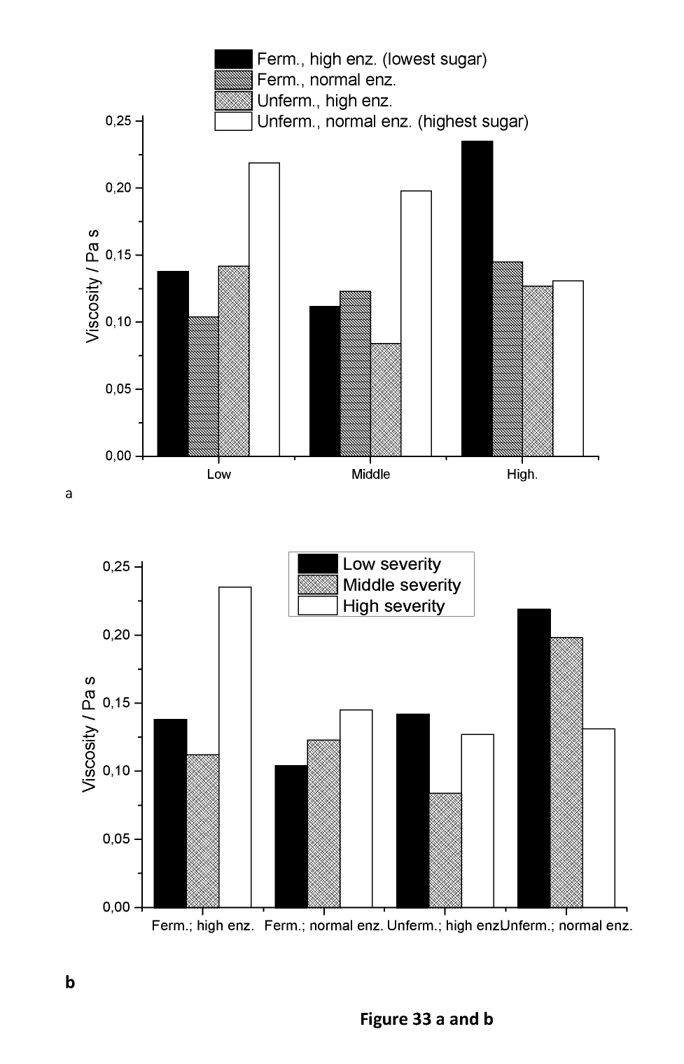

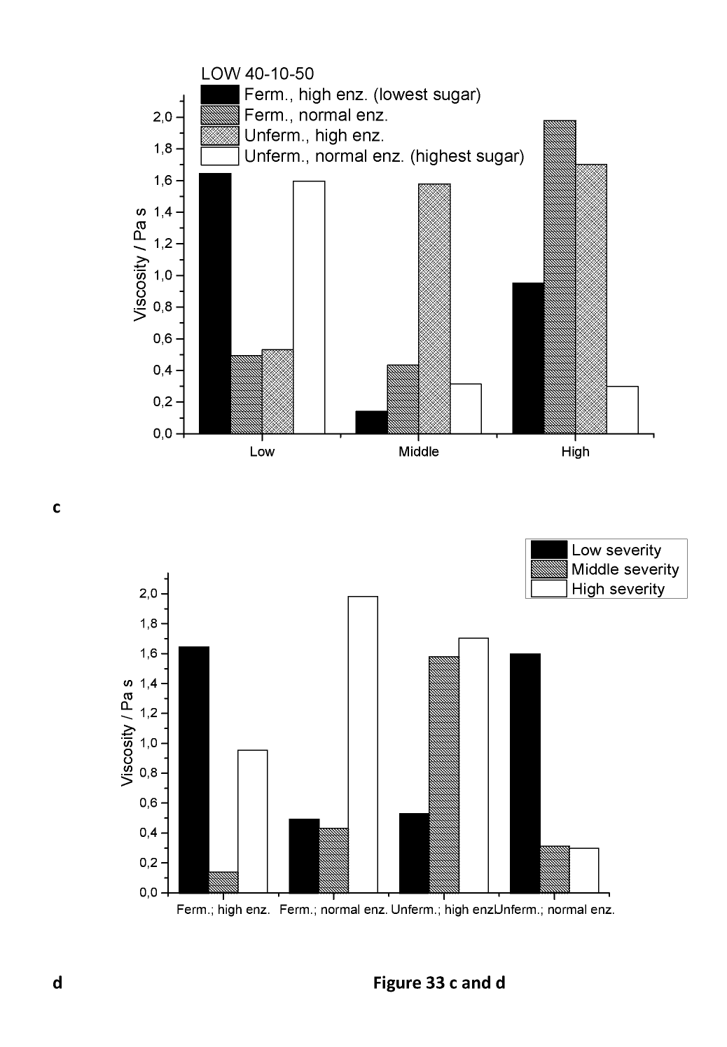

Based on the assumption that sugar decreases viscosity, it would be expected that formulations with high severity, high enzyme dosage and/or with fermented lignin samples should be the most viscous. In FIG. 33 viscosity measurements are shown for the twelve samples; divided into groups based on pretreatment severity (21-4a) and hydrolysis fermentation conditions (21-4b). No clear trends are observed. For the LOW 30-20-50 formulations prepared from “high severity” lignin, viscosity decreases with increasing sugar content (as predicted), but the opposite is observed for the “low” and “middle” severity conditions. For the unfermented samples produced with normal enzyme dosage (ie with the highest sugar content) viscosity increases with increasing xylan number (ie decreasing pretreatment severity), contrary to the prediction. On the other hand, for the fermented samples with normal enzyme dosage (ie the “standard” Inbicon conditions), viscosity increases with decreasing xylan number (ie increasing pretreatment severity).

A similar chaotic behaviour is observed for the LOW 40-10-50 formulations.

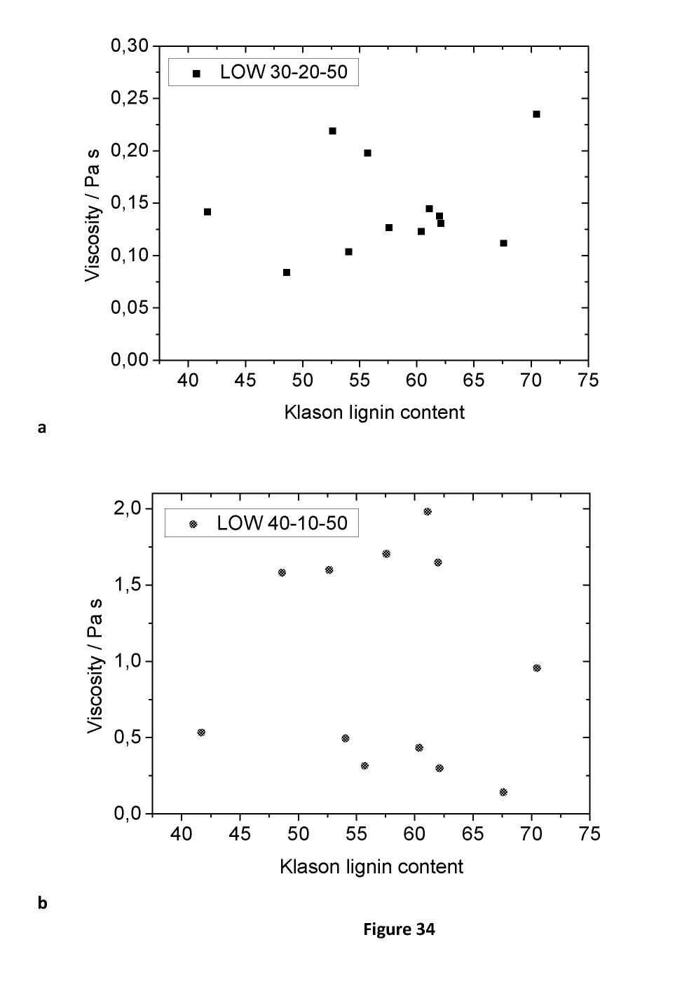

Finally, in FIG. 34, viscosity is depicted as a function of klason lignin for both sets of formulations, without any clear connection being observed.

TABLE 21-3
Details of the lignin samples

| Name | Severity | Hydrolysis | Fermentation | Glucan | Xylan | Klason lignin | Viscosity* |
|---|---|---|---|---|---|---|---|
| 13-62-R6 F1 | Low (13.5%) | Normal | Yes | 8.45% | 6.64% | 54.07% | 0.104 |
| 13-62-R6 F3 | High (4.6%) | Normal | Yes | 14.80% | 1.20% | 61.11% | 0.145 |
| 13-62-R6 F2 | Low (13.5%) | High | Yes | 11,.12% | 5,.56% | 61,.99% | 0,.138 |
| 13-62-R6 F4 | High (4.6%) | High | Yes | 7.64% | 1.17% | 70.48% | 0.235 |
| 13-62-R6 F5 | Middle (9.1%) | Normal | Yes | 8.32% | 3.01% | 60.40% | 0.123 |
| 13-62-R6 F6 | Middle (9.1%) | High | Yes | 3.42% | 2.87% | 67.61% | 0.112 |
| 13-62-R6 1 | Low (13.5%) | Normal | No | 20.25% | 4.19% | 41.72% | 0.142 |
| 13-62-R6 2 | Low (13.5%) | High | No | 14.16% | 4.16% | 52.67% | 0.219 |
| 13-62-R6 3 | High (4.6%) | Normal | No | 15.42% | 1.74% | 57.57% | 0.127 |
| 13-62-R6 4 | High (4.6%) | High | No | 10.48% | 0.87% | 62.12% | 0.131 |
| 13-62-R6 5 | Middle (9.1%) | Normal | No | 17.35% | 2.82% | 48.64% | 0.084 |
| 13-62-R6 6 | Middle (9.1%) | High | No | 12.71% | 2.84% | 55.71% | 0.198 |

*Viscosity (in Pa s) of a LOW 30-10-50 with 5000 ppm Lutensol AP10 and sodium benzoate; measured at room temperature at shear rate 100 s⁻¹.

In conclusion; although a relation between viscosity and sugar content was initially suspected, based on experiments with a specific lignin material being diluted with an alternative low-sugar material, designing different lignin materials with different sugar content did not reveal any clear relation between sugar content and viscosity.

### Example 22

### Acid Wash

Grinded lignin pellets were suspended in a ^{˜}1 M solution of HCl (pH=1). The pellets were washed repeatedly with water until pH ^{˜}5. Lignin was then separated through filtration; DM of the filtered material was 79%. The material was then dried to DM 100%.

Simultaneously, grinded lignin pellets were suspended in concentrated NH4OH (pH=10), washed until pH=8, and filtered. The filtration was quite difficult, with the filter getting clogged easily. This resulted in material with DM 56%, which had a very unpleasant, ammonia-like smell. The material was dried to DM 100%.

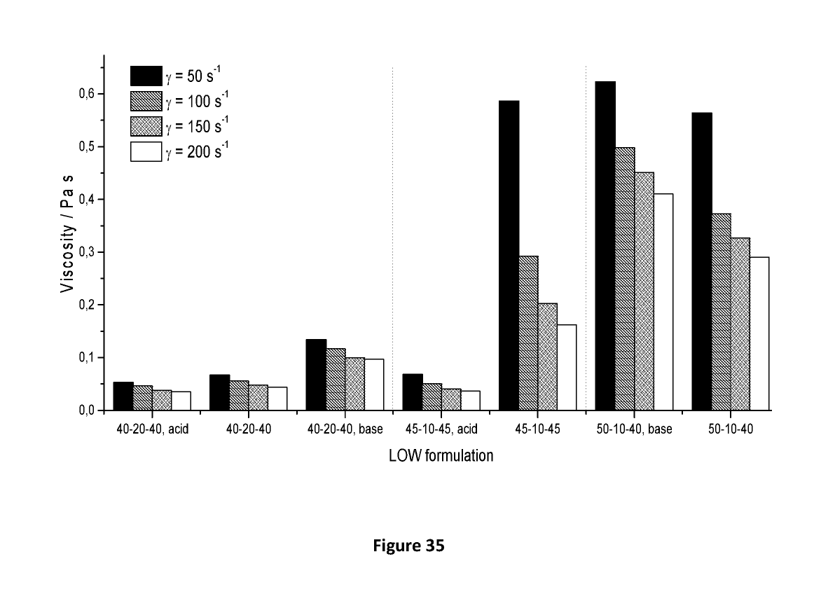

Different LOW formulations were prepared with the acid treated lignin, which was compared to formulations prepared with untreated lignin, shown in FIG. 35 and Table 22-1. In the 40-20-40 formulation. Whereas the base treated lignin did not result in lower viscosity Lignomulsion, acid treated lignin resulted in significant decreases in viscosity of the two tested formulations.

TABLE 22-1
Formulations of acid-, base- and untreated lignin material

| Name | Lignin type | L | O | W | AP10 | SoBenz |
|---|---|---|---|---|---|---|
| 140331_001 | Acid | 40 | 20 | 40 | 0.5 | 0.5 |
| 130312_001 | Untreated | 40 | 20 | 40 | 0.5 | 0.5 |
| 140331_003 | Base | 40 | 20 | 40 | 0.5 | 0.5 |
| 140331_002 | Acid | 45 | 10 | 45 | 0.5 | 0.5 |
| 130307_002 | Untreated | 45 | 10 | 45 | 0.5 | 0.5 |
| 140331_004 | Base | 50 | 10 | 40 | 0.5 | 0.5 |
| 130627_001 | Untreated | 50 | 10 | 40 | 0.5 | 0.5 |

### Example 23

### Ultraturrax Power Input Study

Based on visual inspection, there was an expectation that the duration of mixing LOW formulations with an ultra turrax (UT) affected emulsion viscosity, and experiments were designed to quantify this effect.

LOW 40-20-40 formulations (with 5000 ppm sodium benzoate and 5000 ppm Lutensol AP10) were prepared and mixed with the Ultra Turrax (UT). The lignin came from dried, grinded pellets and the oil was diesel oil. UT speed and duration was varied. Energy consumption of the UT was estimated with an “energy-meter”. This number was compared to viscosity, measured at four different shear rates (50 s⁻¹, 100 s⁻¹, 150 s⁻¹, 200 s⁻¹), all at room temperature. Details are shown in Table 23-1:

TABLE 23-1
Energy input and viscosity of LOW 40-20-40 formulations for first run of experiments

| | | | | Viscosity | | | |
|---|---|---|---|---|---|---|---|
| | UT time | UT speed | Energy | Pa s | | | |
| ID | min | rpm | J | 50 s⁻¹ | 100 s⁻¹ | 150 s⁻¹ | 200 s⁻¹ |
| 130624_002 | 0.5 | 10.000 | 1704 | 0.107 | 0.086 | 0.079 | 0.075 |
| 130624_003 | 1 | 10.000 | 3331 | 0.175 | 0.124 | 0.107 | 0.099 |
| 130624_004 | 10 | 10.000 | 28760 | 0.318 | 0.204 | 0.166 | 0.150 |
| 130624_005 | 0.5 | 3.500 | 895 | 0.208 | 0.148 | 0.123 | 0.111 |
| 130624_006 | 0.5 | 5.000 | 1200 | 0.246 | 0.193 | 0.161 | 0.153 |
| 130624_007 | 0.5 | 20.000 | 3522 | 0.208 | 0.117 | 0.100 | 0.095 |
| 130709_004 | 10 | 3.500 | 17295 | 0.154 | 0.121 | 0.121 | 0.126 |
| 130709_005 | 10 | 5.000 | 19603 | 0.194 | 0.146 | 0.136 | 0.134 |
| 130710_003 | 10 | 10.000 | 33410 | 0.207 | 0.145 | 0.123 | 0.107 |
| 130710_004 | 10 | 20.000 | 56583 | 0.799 | 0.454 | 0.316 | 0.274 |

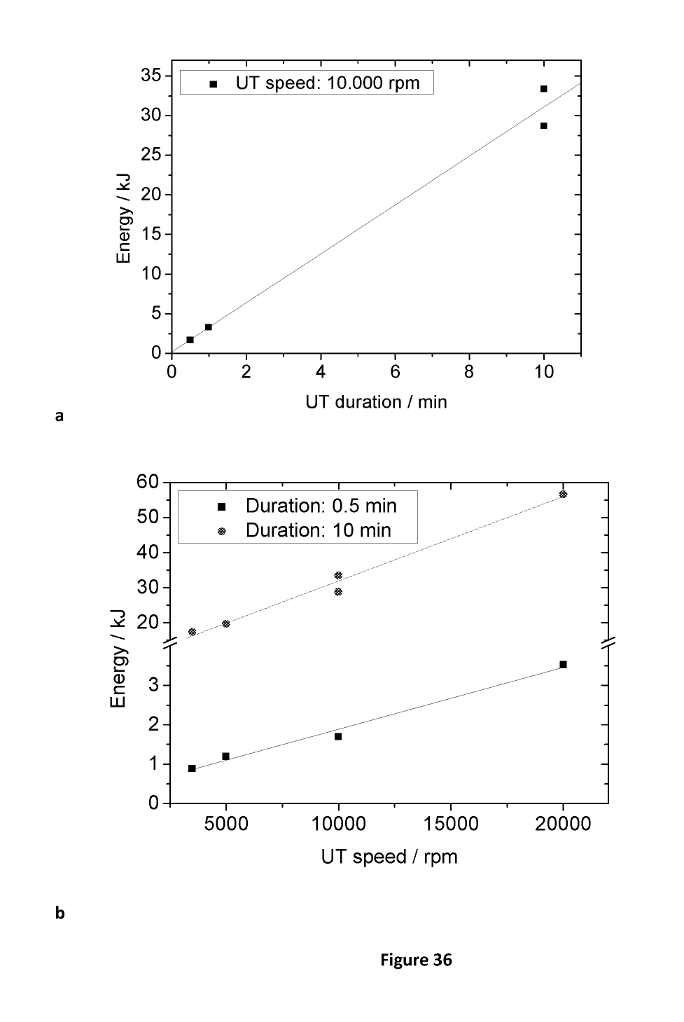

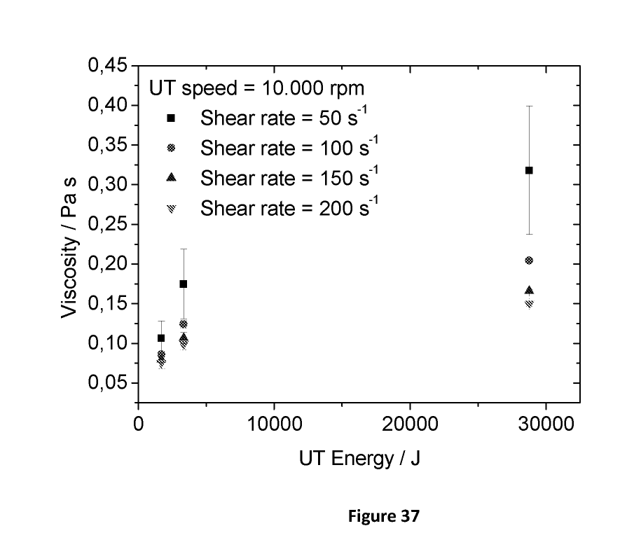

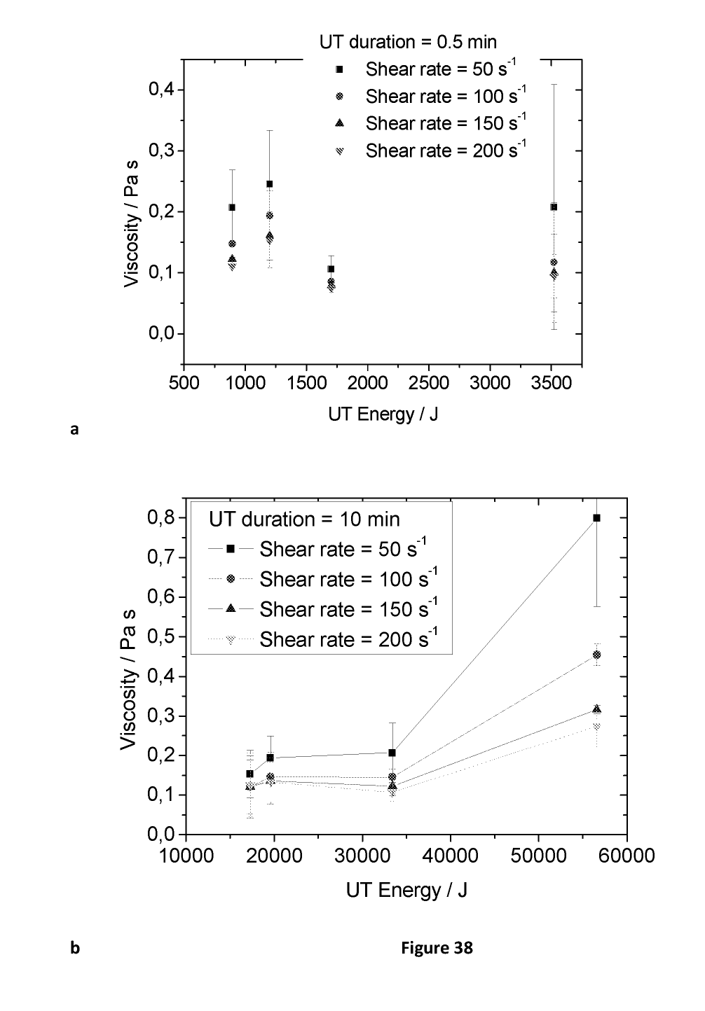

FIG. 36 shows how the energy input is related linearly to ultra turrax duration and speed, respectively. Viscosity is shown as a function of the UT energy input in FIGS. 37 and 38.

FIG. 37 shows a clear dependence between emulsion viscosity and UT duration at the four shear rates, ie that when UT duration is increased, the emulsion becomes more viscous. The effect does not seem linear; it could be for low durations and then reaching a constant level.

FIG. 38 shows viscosity as a function of UT speed at two different durations, 0.5 min and 10 min. At 0.5 min, no dependency between emulsion viscosity and UT speed is observed, but this is probably due to the short time scale and low energy input (resulting in very little variation). At a duration of 10 min., a clear correlation between viscosity and the ultra turrax effect is seen.

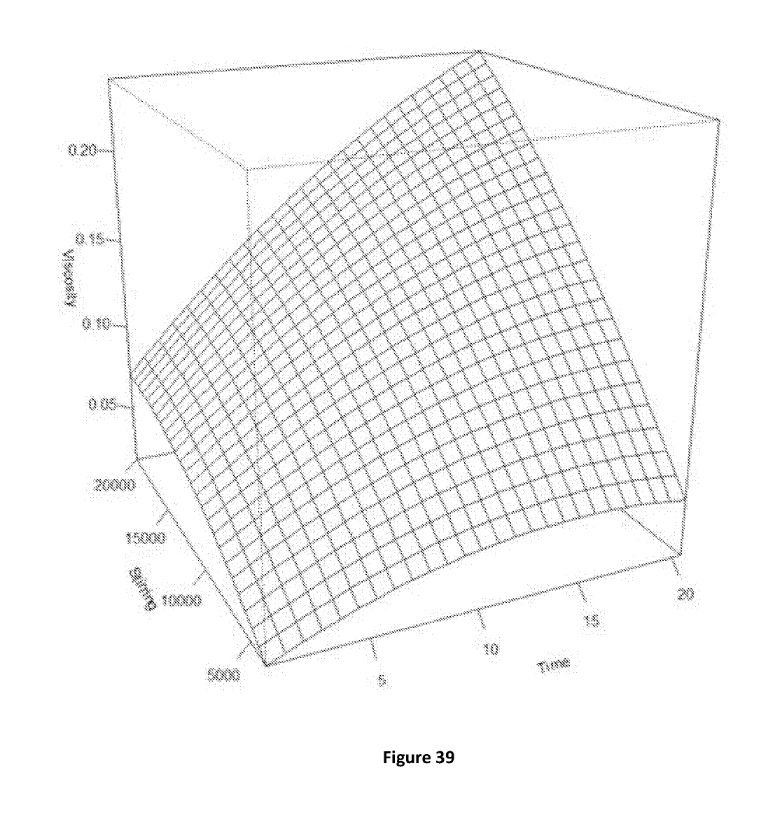

In conclusion, increasing UT duration from 0.5 min to 10 min (ie a factor of 20) increases viscosity at shear rate 100 s⁻¹ with a factor of 2.4. Since low viscosity and low UT energy consumption are desired, the UT duration should be as short as possible, only long enough to ensure homogeneity of the emulsion. An effect of UT speed was also observed; increasing the UT speed from 3500 to 20000 rpm (a factor of 5.7) increases viscosity with a factor of 3.8. To test which of the factors—duration or speed—was the more significant, a new set of experiments was designed, see Table 23-2. On the basis of these results, FIG. 39 is constructed, showing viscosity as a function of the combined effects of speed (“stirring”) and duration (“time”). Within the tested conditions (duration 0.5-20.5 min; speed 3500-20000 rpm), the minimum is seen at the lowest possible energy input (ie at 3500 rpm for 0.5 min). This may, however, not be enough to ensure a homogenous liquid—especially when working with wet lignin filter cake, which has not been pre-grinded, and has to be milled under wet conditions.

Within the tested conditions, the two factors—duration and speed—seem to be of equal importance.

TABLE 23-2
Energy input and viscosity of LOW 40-20-40 formulations for second
run of experiments

| | | | | Viscosity |
|---|---|---|---|---|
| | UT time | UT speed | Energy | Pa s |
| ID | min | rpm | J | 100 s⁻¹ |
| 20140306_001 | 0.5 | 3500 | 1070.6 | 0.030 |
| 20140306_002 | 15.5 | 15875 | 75338.9 | 0.153 |
| 20140306_003 | 0.5 | 3500 | 1334.7 | 0.029 |
| 20140310_002 | 20.5 | 20000 | 133173.9 | 0.227 |
| 20140310_003 | 20.5 | 20000 | 123947.1 | 0.233 |
| 20140310_004 | 0.5 | 20000 | 9156.6 | 0.078 |
| 20140317_001 | 5.5 | 15875 | 35883.3 | 0.134 |
| 20140317_002 | 5.5 | 7625 | 15096.1 | 0.044 |
| 20140317_003 | 10.5 | 11750 | 41072.4 | 0.132 |
| 20140317_004 | 20.5 | 3500 | 27701.4 | 0.044 |
| 20140317_005 | 20.5 | 11750 | 71361.6 | 0.190 |
| 20140317_006 | 10.5 | 20000 | 77471.7 | 0.198 |
| 20140317_007 | 10.5 | 3500 | 16099.1 | 0.037 |
| 20140317_008 | 20.5 | 3500 | 28428.5 | 0.048 |
| 20140317_009 | 15.5 | 7625 | 35326.6 | 0.101 |
| 20140317_010 | 0.5 | 20000 | 6964.2 | 0.055 |
| 20140317_011 | 0.5 | 11750 | 3378.7 | 0.037 |
| 20140317_012 | 10.5 | 11750 | 39174.1 | 0.127 |

### Example 24

### Stability

Stability was studied in two different ways. In the first case, different lignomulsion formulations were exposed to high G-forces in a centrifuge, and the degree of phase separation was quantified. In the second case, lignomulsion was stored at room temperature or at 5° C. for several months, and viscosity was measured.

### Storage Stability

Most of the formulations presented in the sections above were stored for some time; then viscosity was measured again. If the formulations contained a preserving agent (sodium benzoate or paraben), they were stored at room temperature. Otherwise, they were stored at 5° C. In all emulsions, phase separation was observed after some days of storage. But even after several months of storage, the formulations containing Inbicon lignin (in contrast to Indulin) were easily re-homogenized by manual shaking. It was not necessary to use the ultra turrax or other forms of high-shear mixing.

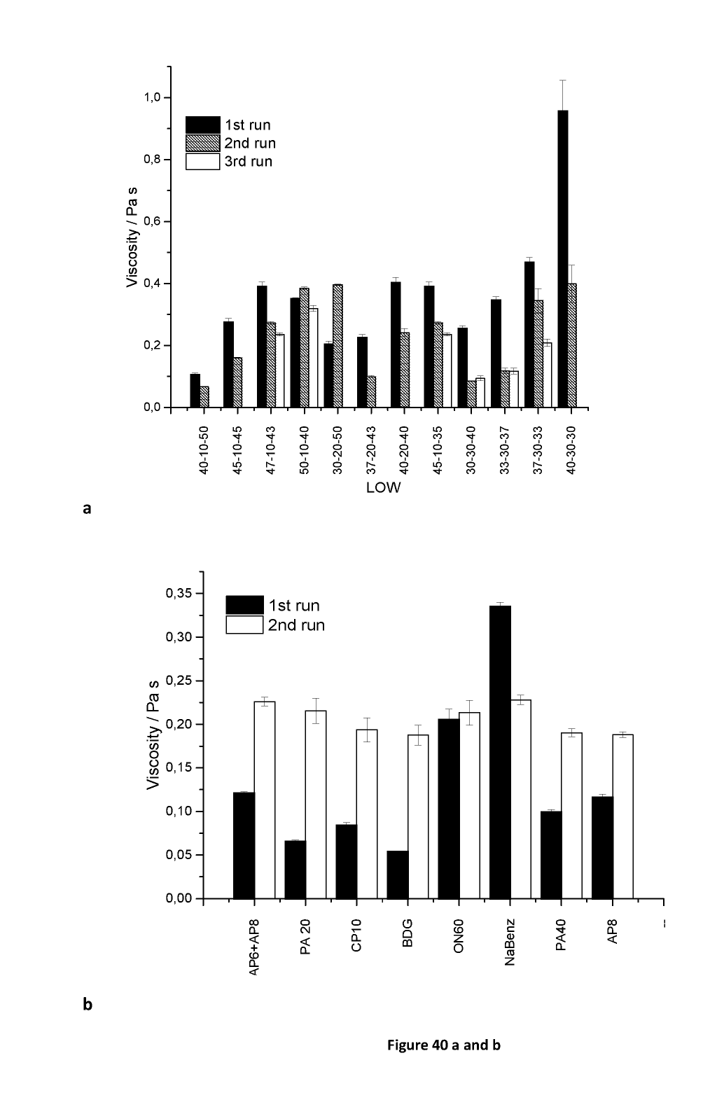

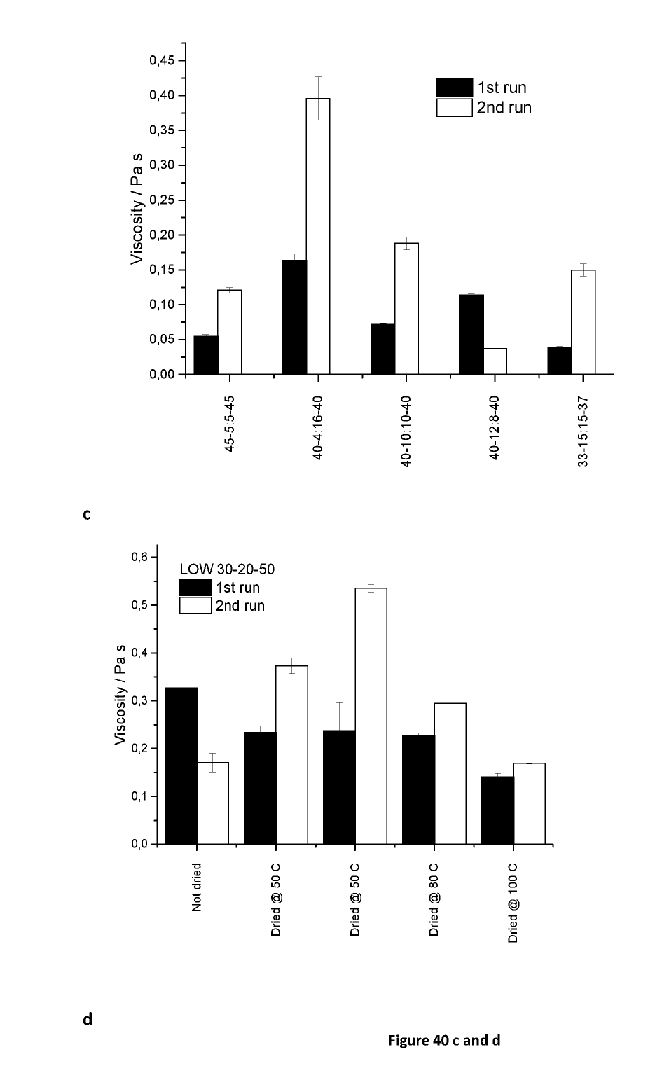

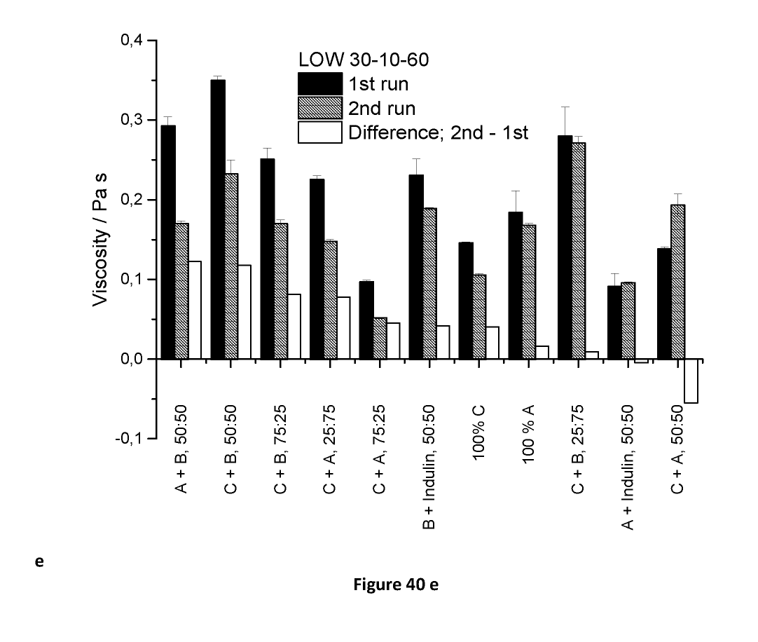

Formulations with grinded lignin pellets, diesel oil and water (in the absence of any additives) were prepared. In general, there is good correspondence between the viscosity measurements before and after the storage period, see FIG. 40a. In most cases, storage causes viscosity to decrease. This would therefore not be a problem with the “pumpability” of Lignomulsion—however, as no surfactants were added in these experiments, they are not representative for the final Lignomulsion formulation.

Formulations with grinded pellets, diesel oil, water, and different hydrotropes and surfactants were prepared. These were stored for approximately 3 months at room temperature. In all studied cases, storage increases viscosity measured at 25° C., see FIG. 40b. However, at temperatures of 45-65° C. the viscosity is the not changed by the storage.

Formulations were prepared using grinded pellets, diesel oil, fuel oil and water and Lutensol AP10 and sodium benzoate. Emulsions were stored for approximately 3 months at room temperature. In some studied cases, storage increases viscosity measured at 25° C., and in others it increases viscosity. There does not seem to be any relation between the viscosity changes and oil content.

Formulations prepared with different types of filter cake, diesel oil, water, Lutensol AP10 and sodium benzoate were prepared and stored at room temperature. For example, the filter cake samples which were 2 dried at different temperatures are shown in FIG. 40d. The “not dried” sample (LOW 30-20-50) shows decrease in viscosity due to the storage period, whereas it increases for the dried samples. On the other hand, the LOW 30-10-60 formulations (see FIG. 40e) all show decreases in viscosity over time, although it is unclear why this is the case. The lignin samples had all been dried to >99% dry matter, and based on the results from 24-1d a decrease would have been expected. The difference could be related to the difference in oil content (although a relation between oil content and viscosity changes were not detected in FIG. 40a and -c).

### Example 25

### Determining Particle Size Distribution

Size distributions of particles in emulsions made from lignin filter cakes and water, prepared by “milling” the lignin while wet with an ultraturrax, were characterized, to determine any correlation between particle size and ultraturrax effect (ie time).

Approximately 350 g lignin filter cake was taken from freezer (dry matter content ^{˜}50%). The cake was manually broken into smaller pieces and placed in the vegetable chopper of the Kenwood. The cut filter cake was then mixed with water and milled with an ultra turrax, operated at 10.000 rpm. Four different samples were prepared with different ultra turrax time: Sample 001 with 1 min, 002 with 5 min, 003 with 10 min and 004 with 30 min.

Particle sizes were measured with a Malvern Mastersizer at Asnæsværket. In the instrument, particles (administered either as a dry powder or an emulsion) were suspended in either water or ethanol. Particle size and concentration were determined from laser diffraction.

For comparison, particle size distributions were also determined from samples of grinded lignin pellets (dry matter content ^{˜}5%) collected with different mesh sizes.

An overview of the measurements is given in table 25-1.

TABLE 25-1

| | | Ultraturrax time/ | | | | |
|---|---|---|---|---|---|---|
| Nr | Lignin type | min | Mesh size | Liquid | Phase | Ultra sound |
| 1 | ISK 12-R6-44 | 1 min | — | Water | Dispersion | No |
| 2 | ISK 12-R6-44 | 5 min | — | Water | Dispersion | No |
| 3 | ISK 12-R6-44 | 10 min | — | Water | Dispersion | No |
| 4 | ISK 12-R6-44 | 30 min | — | Water | Dispersion | No |
| 5 | ISK 12-R6-44 | 1 min | — | Ethanol | Dispersion | No |
| 6 | ISK 12-R6-44 | 30 min | — | Ethanol | Dispersion | No |
| 7 | Grinded pellets | — | <150 | Water | Solid powder | 60 s |
| 8 | Grinded pellets | — | 150 | Water | Solid powder | 60 s |
| 9 | Grinded pellets | — | 250 | Water | Solid powder | 60 s |
| 10 | Grinded pellets | — | 710 | Water | Solid powder | 60 s |
| 11 | Grinded pellets | — | <150 | Ethanol | Solid powder | 60 s |
| 12 | Grinded pellets | — | 250 | Ethanol | Solid powder | 60 s |
| 13 | Grinded pellets | — | 150 | Water | Solid powder | 60 s |
| 14 | Grinded pellets | — | 150 | Water | Solid powder | 60 s |

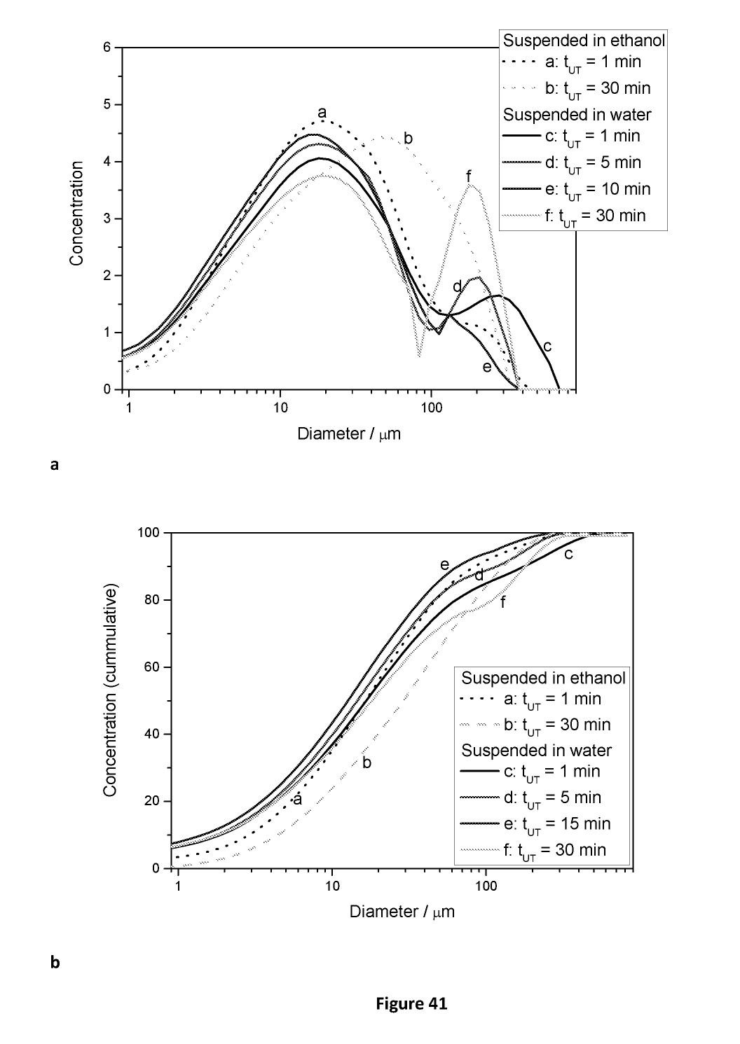

FIG. 41 shows number distributions of wet milled filter cake. The maximal concentration is found below 100 μm, but the shoulder found above 100 μm means that larger particles makes the major contribution to the mass (data not shown). No direct correspondence between particle size and ultra turrax time is observed.

In some cases, ethanol was used as the suspension medium in the instrument. As seen in FIG. 41, there is a quite large difference in the resulting particle size. This is partially explained by the fact that the particles can take up liquid and swell to a bigger size, and partially by the fact that material can be extracted in the liquid phase. This was especially relevant for ethanol, which clearly changed to a darker colour after the addition of lignin.

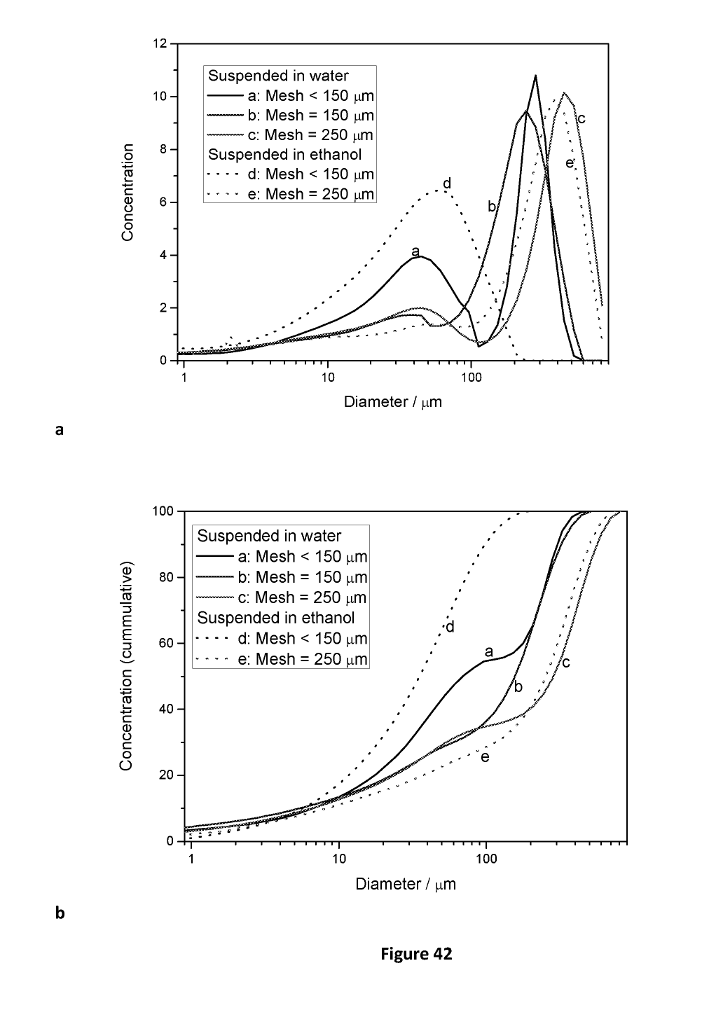

FIG. 42 shows grinded pellets separated into different sizes by sieving, also showing the effect of suspension in water contra ethanol. More importantly, the figure does not show any clear connection between the dry particle size and the particle size measured by the instrument. This is mainly due to swelling of the particles, clouding the difference in dry size. Comparison with FIG. 41 demonstrates the efficiency of wet milling, as a higher proportion of the particles are found below 100 μm in FIGS. 5.3-1b than in FIG. 42.

### Example 26

### Burner Tests

In a very exiting set of experiments, it was tested if lignomulsion could actually ignite and burn. Three different emulsions containing lignin (L), diesel oil (O) and water W were prepared with the following contents: 38:30:32-48:20:32 and 50:30:20, also containing 5000 ppm of sodium benzoate and 5000 ppm of Lutensol AP10. Due to lack of time, only the first two emulsions were tested.

The first emulsion (38:30:32) was sieved through mesh 0.5 mm. However, due to some unfortunate, experimental circumstances the sieving process was slow, and a fraction lignin particles agglomerated to some rather large clumps (diameter of ^{˜}5 cm). The actual composition of this emulsion is therefore unknown. The second and third emulsions (48:20:32 and 50:30:20) was sieved through mesh 1 mm. This sieving went much faster, and lignin agglomeration was negligible.

In the first few burning experiments, the emulsions were heated to 80° C. However, this was actually unnecessary, and it was not done in the last (and most successful, see below) burner experiments.

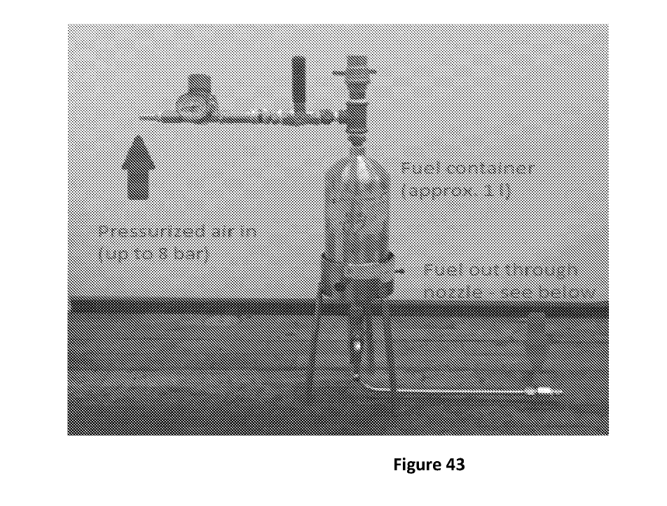

FIG. 43 shows the system for injecting the fuel: Lignomulsion was placed in an approximately 1 l container and using pressurized air (up to 8 bar) it was ejected through a nozzle into a combustion chamber. Initially, the lignomusion was ignited using a weed burner.

Different nozzles were used with varying degrees of success: The first nozzle used was a hollow cone nozzle (RTX 0250 B1) which had too small inner dimensions and was quickly clogged. A homemade nozzle (no name) could not atomize the fuel, and also clogged easily. The three last nozzles (flat and full jet nozzles, respectively) were, however, all capable of administering the fuel well enough for ignition and combustion.

The first emulsion tested was the 38:30:32 formulation using a RTX 0250 nozzle with a nozzle diameter of 1 mm. However, the inner dimensions of the nozzle were significantly smaller and the nozzle clogged within seconds. No real flames were seen in the combustion chamber.

A larger nozzle (2 mm) of the same type was also used, but with the same negative result. Three homemade “nozzles” (basically just tiny holes with diameters of 1-2 mm) were then used. They did not clog as easily, but did not atomize the fuel either. Instead it was “shot” out of the combustion chamber—like water through a hose.

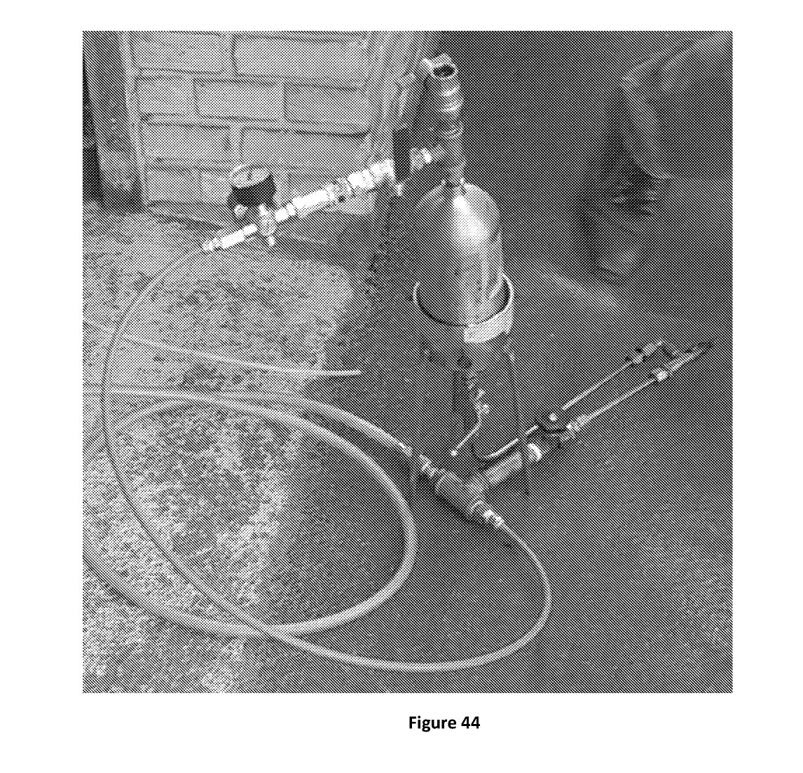

Then, flat jet nozzles were used. First, KGW 1120 and 1190 was used. Initially, it atomized the fuel nicely and flames were seen, but the nozzle also had a tendency to clog. Instead the full cone nozzle (FEEBQ1550B3-“pigtail nozzle”), resulting in the first truly successful burn. The remaining experiments were conducted on the emulsion with the composition 48-20-32. Finally, a modified injection system was designed, using the full cone nozzle, and adding a secondary stream of air, see FIG. 44. This resulted in the best atomization and the most stable combustion.

While experimenting with several different nozzles, the inside of the combustion chamber was repeatedly coated with lignomulsion. This was on several occasions ignited and burned independently for some time.

Using this setup, different Lignomulsion formulations were tested, see Table 26-1 below:

| Nr | Lignin type | Oil type | Lignin | Oil | Water | NaBenz | AP10 | AP8 | Name |
|---|---|---|---|---|---|---|---|---|---|
| 1 | Pellets | Diesel. 10% | 50 | 10 | 40 | 0.5 | 0.5 | 0 | 130702_002 |
| 2 | Pellets | Diesel. 15% | 50 | 15 | 35 | 0.5 | 0.5 | 0 | 130702_003 |
| 3 | Pellets | 1:1 Diesel:Fuel oil | 40 | 20 | 40 | 0.5 | 0.5 | 0 | 130702_004 |
| 4 | Filter cake | Diesel. 20% | 30 | 20 | 50 | 0.5 | 0.5 | 0 | 130701_001 |
| 5 | Indulin | Diesel | 40 | 15 | 44.8 | 0 | 0.1 | 0.1 | 130701_005 |

None of the formulations were neither sieved nor heated prior to combustion.

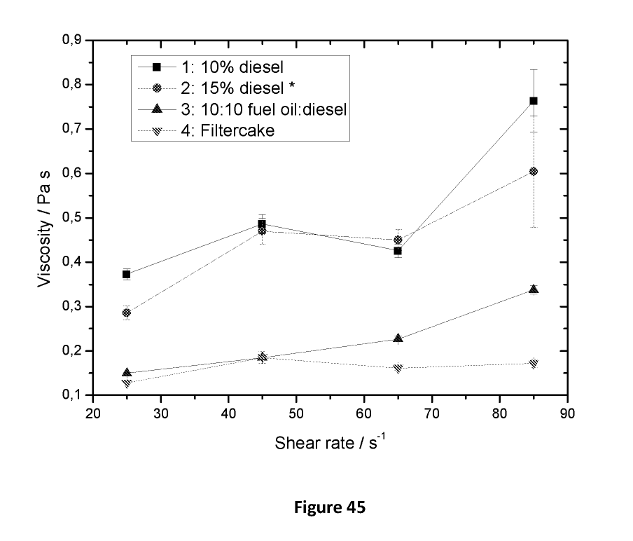

Since a low viscosity is essential for successfully pumping the fuel, an initial study of the sample viscosities was performed and presented below. FIG. 45 shows viscosity at shear rate 100 s−1 as a function of temperature for the first four formulations from Table 26-1 (note, however that the formulation 2 used in the viscosity test had a slightly higher water content—LOW 48-15-37—than the emulsion used in the burner test—LOW 50-15-35). Formulation 5 (with Indulin) was strongly thixotropic. Under static conditions, it was almost solid, but with a lot of stress from shaking, stirring and ultra turrax'ing the viscosity decreased to a degree where it could actually be poured.

Testing of the five formulations is described below.

### 10% Diesel

The formulation was pumped and atomized in the setup, but could not burn even with auxiliary firing—10% diesel is apparently below the lower limit.

Since the formulation could be pumped, a viscosity of ^{˜}0.4 Pa s (slightly) below the upper limit for pourability.

### 15% Diesel

This emulsion was quite viscous and was not very well atomized, however it did seem to burn well. In a future experiment, a little lignin should be replaced with water—or the viscosity should be lowered with viscosity modifying additives. Alternatively, the formulation may atomize better on a more optimized setup.

### Fuel Oil

It has previously been demonstrated that a formulation with 20% diesel burns well. We therefore replaced half of the diesel with heavy fuel oil, but the emulsion did not burn without auxiliary firing. It should probably have been preheated.

### Filter Cake

The formulation was prepared using filter cake, which had been dried to >90% DM and wet milled with an ultra turrax. However, the formulation contained some large particles which clogged the nozzle. The emulsion was therefore not successfully pumped and atomized—and consequently did not burn.

It is believed that such problems could be overcome, e.g. by a more careful wet milling, and avoiding larger particles, i.e. particles of a size close to the nozzle size, or somewhat smaller, could be avoided, e.g. by including a size separation step such as sieving. Alternatively, or in combination, larger nozzles should be used.

Emulsion with Indulin

It has been demonstrated and documented that lignomulsion burns very well. However, choosing the injection system is not trivial. Pumping the fuel with pressured air works very well, and full cone and flat jet nozzles work well. Ensuring sufficient air during combustion appears also important.

### REFERENCES

Posarac, D. and Watkinson, A. “Mixing of a lignin-based slurry fuel,” The Canadian Journal of Chemical Engineering (2000) 78:265
Thammachote, N. “Combustion of lignin mixtures in a rotary lime kiln,” Pulp & Paper Canada (1996) 97(9):51

1. A fluid composition comprising a lignin component, an organic fraction in liquid state at 25° C., and optionally water and/or a further agent.

2. A fluid composition according to claim 1, wherein said lignin component is not lignin from paper and pulp production, such as Kraft lignin, wherein said Kraft lignin being provided from biomass by a process known in the art as Kraft process/method.

3. A fluid composition according to claim 1 or 2, wherein said lignin component has not been provided by a Kraft method or another method comprising an alkaline treatment, such as by addition of NaOH or another base to provide a pH of around 10 or higher, at or around pH 11 or higher, or at or around pH 12 or higher.

4. A fluid composition according to any one of the preceding claims, wherein said lignin component has not been esterified and/or subjected to an esterification step, such as disclosed in WO2015/094098.

5. A fluid composition according to any one of the preceding claims, wherein said lignin component has a Lignin Ion Exchange Capacity (LIEC) of 0.3 mol/kg dry matter (DM) or less, such as 0.25 mol/kg DM or less, such as 0.20 mol/kg DM or less, such as 0.15 mol/kg DM or less, or such as 0.10 mol/kg DM or less.

6. A fluid composition according to any one of the preceding claims, wherein said lignin component has a LIEC in the range of 0.05-0.30, 0.10-0.25, or 0.10-0.15 mol/kg DM.

7. A fluid composition according to any one of the preceding claims, wherein said lignin component is significantly less polar than Kraft lignin, such as assessed by LIEC measurement, such as having a LIEC at least 0.10, 0.11, 0.12, 0.13, 0.14, 0.15, 0.16, or 0.17 mol/kg DM lower than the LIEC of Kraft lignin.

8. A fluid composition according to any one of the preceding claims, wherein said lignin component is significantly less hygroscopic, such as binding at least 20, 25, 30, 35, 40, 45, 50, 55, 60, 65, 70, 75, 80, 85, 90, 95 or 100% (w/w) less water when compared to Kraft lignin.

9. A fluid composition according to any one of the preceding claims, wherein said lignin component is swelling significantly less than Kraft lignin, such as swelling at least 20, 25, 30, 35, 40, 45, 50, 55, 60, 65, 70, 75, 80, 85, 90, 95 or 100% less, and optionally wherein said swelling is determined as change in particle size upon suspension in water or another suitable medium after 60 min.

10. A fluid composition according to any one of the preceding claims, wherein said fluid composition is significantly more stable and/or pumpable when compared to a similar composition prepared with Kraft lignin.

11. A fluid composition according to claim 10, wherein the pumpability is defined as having a viscosity of 1 Pa·s or less, such as 0.9 Pa·s or less, such as 0.8 Pa·s or less, such as 0.7 Pa·s or less, such as 0.6 Pa·s or less, such as 0.5 Pa·s, such as 0.4 Pa·s or less, such as 0.3 Pa·s or less, such as 0.2 Pa·s or less, or such as 0.1·Pa·s or less at a shear rate of 100 s⁻¹, wherein said viscosity is being measured as average over a time period of 10 min.

12. A fluid composition according to any one of the preceding claims, wherein said fluid composition possesses a significant short term, medium term, or long term stability and/or pumpability, wherein said short, medium, and long term are periods of time in the range of 1-60 min, >1-24 h, or >24 h, respectively.

13. A fluid composition according to any one of the preceding claims, wherein said fluid composition has an increased short term, medium term and/or long-term stability and/or pumpability, when compared to a similar composition prepared with Kraft lignin.

14. A fluid composition according to claim 12 or 13, wherein said short term time period is 1, 2, 5, 10, 15, 20, 30, 45, or 60 min.

15. A fluid composition according to claim 12 or 13, wherein said medium term time period is 90 min, 2 h, 4 h, 6 h, 8 h, 10 h, 12 h, 18 h, 24 h.

16. A fluid composition according to claim 12 or 13, wherein said long term time period is 25 h, 30 h, 40 h, 2 d, 3 d, 4 d, 5 d, 6 d, 1 week, 2 weeks, 3 weeks, 1 month, 2, months, 3 month, 4 months, 5 months, 6 months, or more than 6 month.

17. A fluid composition according to any one of claims 10-16, wherein said stability is defined in the sense that no more than 5.0, 4.0, 3.0, 2.0, 1.0 or 0.5% (w/w) of any one of the fractions (e.g. water, liquid organic fraction, and/or lignin component) of said fluid composition will separate after said specified period of time.

18. A fluid composition according to any one of the preceding claims, wherein occasional or constant gentle stirring, agitation, and/or re-circulation but no high shear mixing is be required for maintaining said stability and/or pumpability.

19. A fluid composition according to any one of the preceding claims comprising two or more fractions, wherein (a) the first fraction is an organic fraction in liquid state at room temperature, said organic fraction comprising one or more organic compounds such as one or more fat, and/or one or more oil; and (b) the second fraction comprises one or more lignin component.

20. A fluid composition according to any one of the preceding claims comprising 5-60% (w/w) lignin component, 0-40% (w/w) organic fraction, 0-60% (w/w) water, and 0-1.0% (w/w) further agent.

21. A fluid composition according to any one of the preceding claims, wherein the water is comprised (i) in the first fraction, such as an oil- and/or fat-water-emulsion, as a homogenous solution of oil and/or fat and water; (ii) as a third, aqueous fraction; or (iii) as a combination of (i) and (ii).

22. A fluid composition according to any one of the preceding claims, wherein the Lignin Ion Exchange Capacity is around 0.40, 0.35, 0.30, 0.25, 0.20, 0.15, 0.10, mol/kg dry matter or less; in the range of 0.10-0.20, 0.20-0.30, 0.30-0.40 mol/kg dry matter; and/or in the range of 0.05-0.40, 0.10-0.30, or 0.10-0.20 mol/kg DM.

23. A fluid composition according to any one of the preceding claims, further comprising one or more further agent, such as an agent is selected from the group comprising or consisting of one or more dispersing agent(s), surfactant(s), hydrotropic agent(s), emulsifier(s), preserving agent(s), and any combination thereof.

24. A fluid composition according to any one of the preceding claims, wherein said one or more further agent is present in the range of 0.001% to 5% (w/w).

25. A fluid composition according to any one of the preceding claims, wherein the one or more fraction, liquid organic fraction, oil, lignin component, water, further agent, dispersing agent, surfactant, hydrotropic agent, emulsifier, preserving agent, and any combination thereof are in a state of being intermixed.

26. A fluid composition according to claim 25, wherein the state of being intermixed is selected from the group comprising or consisting of being intermixed as a solution; being intermixed as a suspension; being intermixed as an emulsion; being intermixed as a dispersion; being intermixed as a slurry; and any combination thereof.

27. A fluid composition according to any one of the preceding claims, wherein said lignin component comprises cellulose in an amount of 2,000-300,000 ppm, such as 3,000-180,000 ppm, e.g. 4,000-160,000 ppm, for example 5,000-140,000 ppm, such as 6,000-120,000 ppm, 7,000-100,000 ppm, for example 8,000-80,000 ppm, such as 9,000-70,000 ppm, e.g. 10,000-60,000 ppm, 12,000-50,000 ppm, such as 14,000-50,000 ppm, e.g. 16,000-40,000 ppm, 18,000-30,000 ppm, such as 20,000-28,000 ppm, for example 22,000-26,000 ppm (w/w) in relation to said fluid composition.

28. A fluid composition according to any one of the preceding claims, wherein said lignin component comprises hemicellulose in an amount of 2,000-200,000 ppm, such as 3,000-180,000 ppm, e.g. 4,000-160,000 ppm, for example 5,000-140,000 ppm, such as 6,000-120,000 ppm, 7,000-100,000 ppm, for example 8,000-80,000 ppm, such as 9,000-70,000 ppm, e.g. 10,000-60,000 ppm, 12,000-50,000 ppm, such as 14,000-50,000 ppm, e.g. 16,000-40,000 ppm, 18,000-30,000 ppm, such as 20,000-28,000 ppm, for example 22,000-26,000 ppm (w/w) in relation to said fluid composition.

29. A fluid composition according to any one of the preceding claims, wherein said lignin component comprises ash in an amount of 2,000-200,000 ppm, such as 3,000-180,000 ppm, e.g. 4,000-160,000 ppm, for example 5,000-140,000 ppm, such as 6,000-120,000 ppm, 7,000-100,000 ppm, for example 8,000-80,000 ppm, such as 9,000-70,000 ppm, e.g. 10,000-60,000 ppm, 12,000-50,000 ppm, such as 14,000-50,000 ppm, e.g. 16,000-40,000 ppm, 18,000-30,000 ppm, such as 20,000-28,000 ppm, for example 22,000-26,000 ppm (w/w) in relation to said fluid composition.

30. A fluid composition according to any one of claims 23 to 29, wherein said one or more dispersing agent is selected from the group comprising or consisting of non-ionic, anionic, cationic and amphoteric dispersing agent(s) and any combination and/or compatible mixture thereof.

31. A fluid composition according to any one of claims 23 to 30, wherein said dispersing agent is present in said fluid composition in an amount of 10-50,000 ppm or 200-20,000 ppm, such as 300-18,000 ppm, e.g. 400-16,000 ppm, for example 500-14,000 ppm, such as 600-12,000 ppm, 700-10,000 ppm, for example 800-8,000 ppm, such as 900-7,000 ppm, e.g. 1,000-6,000 ppm, 1,200-5,000 ppm, such as 1,400-5,000 ppm, e.g. 1,600-4,000 ppm, 1,800-3,000 ppm, such as 2,000-2,800 ppm, for example 2,200-2,600 ppm (w/w) in relation to said fluid composition.

32. A fluid composition according to any one of claims 23 to 31, wherein said one or more surfactant is selected from the group comprising or consisting of anionic, cationic, zwitterionic and nonionic surfactants, and any combination and/or compatible mixture thereof.

33. A fluid composition according to any one of claim 23 or 32, wherein said surfactant is present in said fluid composition in an amount of 10-50,000 ppm or 200-20,000 ppm, such as 300-18,000 ppm, e.g. 400-16,000 ppm, for example 500-14,000 ppm, such as 600-12,000 ppm, 700-10,000 ppm, for example 800-8,000 ppm, such as 900-7,000 ppm, e.g. 1,000-6,000 ppm, 1,200-5,000 ppm, such as 1,400-5,000 ppm, e.g. 1,600-4,000 ppm, 1,800-3,000 ppm, such as 2,000-2,800 ppm, for example 2,200-2,600 ppm (w/w) in relation to said fluid composition.

34. A fluid composition according to any one of claims 23 to 33, wherein said one or more hydrotrope is selected from the group comprising or consisting of: non-ionic, anionic, cationic and amphoteric hydrotropes and any combination and/or compatible mixtures thereof.

35. A fluid composition according to any one of claims 23 to 34, wherein said hydrotrope is present in said fluid composition in an amount of 10-50,000 ppm or 200-40,000 ppm, such as 300-30,000 ppm, e.g. 400-20,000 ppm, for example 500-15,000 ppm, such as 600-12,000 ppm, 700-10,000 ppm, for example 800-8,000 ppm, such as 900-7,000 ppm, e.g. 1,000-6,000 ppm, 1,200-5,000 ppm, such as 1,400-5,000 ppm, e.g. 1,600-4,000 ppm, 1,800-3,000 ppm, such as 2,000-2,800 ppm, for example 2,200-2,600 ppm (w/w) in relation to said fluid composition.

36. A fluid composition according to any one of claims 23 to 35, wherein said one or more emulsifier is selected from the group comprising or consisting of sodium phosphate(s), sodium stearoyl lactylate cationic, lecithin, DATEM (diacetyl tartaric acid ester of monoglyceride), and any combination and/or compatible mixture thereof.

37. A fluid composition according to any one of claims 23 to 36, wherein said surfactant is present in said fluid composition in an amount of 10-50,000 ppm or 200-20,000 ppm, such as 300-18,000 ppm, e.g. 400-16,000 ppm, for example 500-14,000 ppm, such as 600-12,000 ppm, 700-10,000 ppm, for example 800-8,000 ppm, such as 900-7,000 ppm, e.g. 1,000-6,000 ppm, 1,200-5,000 ppm, such as 1,400-5,000 ppm, e.g. 1,600-4,000 ppm, 1,800-3,000 ppm, such as 2,000-2,800 ppm, for example 2,200-2,600 ppm (w/w) in relation to said fluid composition.

38. A fluid composition according to any one of claims 23 to 37, wherein said preserving agent is selected from the group comprising or consisting of one or more carboxylate, benzoate, benzoic acid derivative such as parabene(s), aldehyde(s), thiazine(s), organic acid(s) and the like, and any combination thereof.

39. A fluid composition according to any one of claims 23 to 38, wherein said preserving agent is present in said fluid composition in an amount of 10-50,000 ppm or 20-10,000 ppm, such as 30-8,000 ppm, e.g. 40-6,000 ppm, for example 50-5,000 ppm, such as 60-4,000 ppm, 70-3,000 ppm, for example 80-2,000 ppm, such as 90-1,500 ppm, e.g. 100-1,200 ppm, 120-1,000 ppm, such as 140-800 ppm, e.g. 160-600 ppm, 180-400 ppm, such as 200-300 ppm, for example 2,200-250 ppm (w/w) in relation to said fluid composition.

40. A fluid composition according to any one of the preceding claims, wherein the dry matter content of said lignin component in said fluid composition is 1.0-99% (w/w), 10-99% (w/w) or 20-95% (w/w), such as 21-94% (w/w), e.g. 22-93% (w/w), such as 23-92% (w/w), such as 24-91% (w/w), for example 25-90% (w/w), such as 26-89% (w/w), such as 27-88% (w/w), for example 28-87% (w/w), e.g. 29-86% (w/w), such as 30-85% (w/w), such as 31-84% (w/w), such as 32-83% (w/w), such as 33-82% (w/w), for example 34-81% (w/w), such as 35-80% (w/w), e.g. 36-79% (w/w), such as 37-78% (w/w), e.g. 38-77% (w/w), e.g. 39-76% (w/w), such as 40-75% (w/w), such as 41-74% (w/w), such as 42-73% (w/w), such as 43-72% (w/w), for example 44-71% (w/w), such as 45-70% (w/w), e.g. 46-69% (w/w), such as 47-68% (w/w), e.g. 48-67% (w/w), e.g. 49-66% (w/w), such as 50-65% (w/w), such as 51-64% (w/w), such as 52-63% (w/w), such as 53-62% (w/w), for example 54-61% (w/w), such as 55-60% (w/w), e.g. 56-59% (w/w), such as 57-58% (w/w).

41. A fluid composition according to any one of the preceding claims, wherein lignin component comprises sulfur and the sulfur content of the dry matter content of said lignin component is 2.0% (w/w) or less, such as 1.4% (w/w) or less, such as 1.3% (w/w) or less, for example 1.2% (w/w) or less, such as 1.1% (w/w) or less, e.g. 1.0% (w/w) or less, such as 0.9% (w/w) or less, for example 0.8% (w/w) or less, such as 0.7% (w/w) or less, e.g. 0.6% (w/w) or less, e.g. 0.5% (w/w) or less, such as 0.4% (w/w) or less, for example 0.3% (w/w) or less, such as 0.2% (w/w) or less, or 0.1% (w/w) or less, such as 0.09% (w/w) or less, such as 0.08% (w/w) or less, e.g. 0.07% (w/w) or less, e.g. 0.06% (w/w) or less, such as 0.05% (w/w) or less, for example 0.04% (w/w) or less, such as 0.03% (w/w) or less, e.g. 0.02% (w/w) or less, such as 0.01% (w/w) or less.

42. A fluid composition according to any one of the preceding claims, wherein said lignin component in said fluid composition is having an average grain size of 1-2000 μm, 1-1500 μm, 1-1200 μm, 1-1000 μm, 1-800 μm, 1-600 μm, 1-500 μm, 1-450 μm, such as 1.5-430 μm, e.g. 2-420 μm, such as 3-410 μm, for example 4-400 μm, e.g. 5-390 μm, such as 6-380 μm, e.g. 7-370 μm, such a 8-360 μm, 9-350 μm, for example 10-340 μm, e.g. 12-330 μm, such as 14-320 μm, such as 16-310 μm, for example 18-300 μm, e.g. 20-290 μm, such as 22-280 μm, e.g. 25-270 μm, such a 30-260 μm, 35-250 μm, for example 40-240 μm, e.g. 45-230 μm, such as 50-220 μm, for example 60-210 μm, for example 70-200 μm, e.g. 80-190, for example 90-180 μm, e.g. 100-170 μm, such a 110-160 μm, 120-150 μm, for example 130-140 μm.

43. A fluid composition according to claim 42, wherein said average grain or particle size is determined before or after providing said fluid composition, and optionally, wherein said grain or particle size being measured by laser diffraction spectroscopy, or e.g. by a Malvern Mastersizer.

44. A fluid composition according to any one of the preceding claims, wherein said lignin component originates from a lignocellulosic biomass having been subjected to a hydrothermal pretreatment followed by a hydrolysis of at least part of the cellulose and at least part of the hemicellulose present in said lignocellulosic biomass.

45. A fluid composition according to any one of the preceding claims, wherein said lignin component originates from a lignocellulosic biomass having been subject to a hydrothermal pretreatment followed by a hydrolysis of at least part of the cellulose and at least part of the hemicellulose present in said lignocellulosic biomass; and optionally followed by a fermentation, such as an alcohol fermentation.

46. A fluid composition according to claim 44 or 45, wherein said hydrolysis is an acid catalyzed hydrolysis, an enzymatic hydrolysis or a combination of acid/enzyme-catalyzed hydrolysis.

47. A fluid composition according to any one of the preceding claims, wherein said lignin component is having an average molecular weight (Da) of 1,000 or above, 1,500 or above, 2,000 or above, 2,500 or above, 3,000 or above, such as 3,500 or above, e.g. 4,000 or above, such as 5,000 or above, for example 5,500 or above, such as 6,000 or above, e.g. 7,000 or above, for example 8,000 or above, such as 9,000 or above, for example 10,000 or above, such as 12,000 or above, e.g. 14,000 or above, for example 16,000 or above, e.g. 18,000 or above, e.g. 20,000 or above, such as 25,000 or above, e.g. 30,000 or above, such as 35,000 or above, for example 40,000 or above, such as 45,000 or above, e.g. 50,000 or above, such as 55,000 or above, e.g. 60,000 or above, such as 65,000 or above, e.g. 70,000 or above, such as 75,000 or above, for example 80,000 or above, such as 85,000 or above, e.g. 90,000 or above, such as 95,000 or above, or 100,000 or above.

48. A fluid composition according to any one of the preceding claims, wherein said lignin component originates from a lignocellulosic biomass obtained from an annual or a perennial plant.

49. A fluid composition according to any one of the preceding claims, wherein said lignin component originates from a lignocellulosic biomass obtained, obtainable or derived from the group comprising or consisting of one or more of: cereal, wheat, wheat straw, rice, rice straw, corn, corn fiber, corn cobs, corn stover, hardwood bulk, softwood bulk, sugar cane, sweat sorghum, bagasse, nut shells, empty fruit bunches, grass, cotton seed hairs, barley, rye, oats, sorghum, brewer's spent grains, palm waste material, wood, soft lignocellulosic biomass, and any combination thereof.

50. A fluid composition according to any one of the preceding claims, wherein said lignin component comprises one or more impurities originating from its mode of production, such as enzyme residues, yeast residues, foam depressant(s), clean in place (CIP) compounds, salts and the like.

51. A fluid composition according to any one of the preceding claims, wherein said lignin component comprises impurity/impurities originating from compounds native for the lignocellulosic material, such as cellulose residues, hemicellulose residues, monomeric sugar compounds, dimeric sugar compounds, oligomeric sugar compounds, carbohydrate residues, wax residues, minerals, ash, silica (SiO₂), silica comprising compositions, salts, organic acids, and the like, and any combination thereof.

52. A fluid composition according to any one of the preceding claims, wherein the purity of said lignin component is 50% (w/w) or more, such as 52% (w/w) or more, for example 54% (w/w) or more, such as 56% (w/w) or more, e.g. 58% (w/w) or more, such as 60% (w/w) or more, such as 62% (w/w) or more, for example 64% (w/w) or more, such as 66% (w/w) or more, e.g. 68% (w/w) or more, such as 70% (w/w) or more, such as 72% (w/w) or more, for example 74% (w/w) or more, such as 76% (w/w) or more, e.g. 78% (w/w) or more, such as 80% (w/w) or more, such as 82% (w/w) or more, for example 84% (w/w) or more, such as 86% (w/w) or more, e.g. 88% (w/w) or more, such as 90% (w/w) or more, such as 92% (w/w) or more, for example 94% (w/w) or more, such as 96% (w/w) or more, e.g. 98% (w/w) or more.

53. A fluid composition according to claim 48, wherein said purity is determined based on content of Klason lignin or acid insoluble lignin, and optionally, wherein the corresponding percentage constituting impurities may be any one or more impurity as defined in claim 50 or 51.

54. A fluid composition according to any one of the preceding claims, wherein the content of said organic fraction in said fluid composition is at least 2, 5, 10, 15, 20, 25, 30, 35, 40, 45, 50, 55, 60, 65, 70, 75, 80, 85, 90, or 95% (w/w) or more, such as 2-95% (w/w), such as 4-78% (w/w), e.g. 6-76% (w/w), such as 8-74% (w/w), e.g. 10-72% (w/w), such as 12-70% (w/w), e.g. 14-68% (w/w), such as 16-66% (w/w), for example 18-64% (w/w), such as 20-62% (w/w), e.g. 22-60% (w/w), for example 24-58% (w/w), such as 26-56% (w/w), such as 28-54% (w/w), such as 30-52% (w/w), 32-50% (w/w), e.g. 34-48% (w/w), such as 36-46% (w/w), such as 38-44% (w/w), for example 40-42% (w/w).

55. A fluid composition according to any one of the preceding claims, wherein said organic fraction comprises or consists essentially of an organic solvent, a distillate, and/or a residue from a hydrocarbon distillation.

56. A fluid composition according to claim 55, wherein said distillate is selected from the group comprising or consisting of one or more mineral oil, kerosene, diesel, No. 2 fuel oil, No. 3 fuel oil, No. 4 fuel oil fuel oil, No. 5 fuel oil, No. 6 fuel oil, and No. 7 fuel oil, and any mixture(s) thereof.

57. A fluid composition according to any one of the preceding claims, wherein said one or more organic compound of said liquid organic fraction is of plant origin or animal origin.

58. A fluid composition according to claim 57, wherein said one or more organic compound of said liquid organic fraction is an oil of plant origin or a fat of animal origin.

59. A fluid composition according to any one of the preceding claims, wherein said one or more organic compound of said liquid organic fraction is an oil originating from pyrolysis of a biomass, such as a cellulosic or lignocellulosic material or wherein said oil is a pyrolysis oil originating from pyrolysis of a lignin component.

60. A fluid composition according to any one of the preceding claims, wherein said one or more organic compound of said liquid organic fraction is an oil originating from pyrolysis of a polymer, such as a synthetic plastic or synthetic elastomer.

61. A fluid composition according to any one of the preceding claims, wherein said one or more organic compound of said liquid organic fraction is selected from the group comprising or consisting of glycerol, biodiesel, synfuel, biomass to liquid (BTL) diesel, gas to liquid (GTL) diesel, coal to liquid (CTL) diesel, and any combination thereof.

62. A fluid composition according to any one of the preceding claims, wherein said one or more organic compound of said liquid organic fraction originates from treatment of a biomass with water and/or other polar liquid(s), such as ethanol or methanol.

63. A fluid composition according to any one of the preceding claims, wherein the said biomass treatment comprises treatment under supercritical conditions.

64. A fluid composition according to any one of the preceding claims, wherein said biomass which has been treated with water or other polar liquid(s) under supercritical conditions is selected from the group comprising or consisting of one or more lignocellulosic material, cellulose, lignin component, and any combination thereof.

65. A fluid composition according to any one of the preceding claims, wherein said liquid organic fraction or compound of said liquid organic fraction is in itself a mixture of two or more such organic substances, such as three or more such organic substances, e.g. four or more such organic substances, such as five or six or more of such organic substances.

66. A fluid composition according to any one of the preceding claims, wherein the sulfur content of said liquid organic fraction, and/or of the one or more organic compound and/or substance of said organic liquid fraction is 5.0% (w/w) or less, such as 4.5% (w/w) or less, for example 4.0% (w/w) or less, such as 3.8% (w/w) or less, e.g. 3.6% (w/w) or less, for example 3.4% (w/w) or less, e.g. 3.2% (w/w) or less, such as 3.0% (w/w) or less, for example 2.8% (w/w) or less, e.g. 2.6% (w/w) or less, for example 2.4% (w/w) or less, e.g. 2.2% (w/w) or less, such as 2.0% (w/w) or less, for example 1.8% (w/w) or less, such as 1.6% (w/w) or less, for example 1.4% (w/w) or less, e.g. 1.2% (w/w) or less, such as 1.0% (w/w) or less, for example 0.8% (w/w) or less, such as 0.4% (w/w) or less, such as 0.2% (w/w) or less, for example 0.1% (w/w) or less, such as 0.08% (w/w) or less, e.g. 0.06% (w/w) or less, such as 0.04% (w/w) or less, e.g. 0.02% (w/w) or less, for example 0.01% (w/w) or less, such as 0.008% (w/w) or less, e.g. 0.006% (w/w) or less, such as 0.004% (w/w) or less, e.g. 0.002% (w/w) or less, such as 0.001% (w/w), such as 800 ppm or less, e.g. 600 ppm or less, such as 400 ppm or less, e.g. 200 ppm or less, for example 100 ppm or less, such as 50 ppm (w/w) or less.

67. A fluid composition according to any one of the preceding claims, wherein said liquid organic fraction, organic compound or substance of said liquid organic fraction is immiscible with water.

68. A fluid composition according to any one of the preceding claims, wherein said fluid composition, organic fraction, one or more organic compound or substance at 25° C. is having a viscosity of 0.0005-10,000 CSt, such as 0.0010-9,000 CSt, e.g. 0.0050-8,000 CSt, for example 0.01-6,000 CSt, for example 0.05-4,000 CSt, such as 0.1-2,000 CSt, e.g. 0.5-1,000 CSt, such as 1.0-800 CSt, e.g. 5.0-600 CSt, such as 10-400 CSt, for example 50-300 CSt, such as 100-200 CSt.

69. A fluid composition according to any one of the preceding claims, wherein said fluid composition, organic fraction, one or more organic compound or substance, wherein said organic substance at 50° C. is having a viscosity of 0.0004-2,000 CSt, such as 0.0010-1,500 CSt, e.g. 0.0050-1,000 CSt, for example 0.01-800 CSt, for example 0.05-600 CSt, such as 0.1-400 CSt, e.g. 0.5-200 CSt, such as 1.0-100 CSt, e.g. 5.0-80 CSt, such as 10-70 CSt, for example 20-50 CSt, such as 30-40 CSt.

70. A fluid composition according to any one of the preceding claims, wherein said fluid composition, organic fraction, one or more organic compound or substance, wherein said organic substance at 75° C. is having a viscosity of 0.0002-200 CSt, such as 0.0001-150 CSt, e.g. 0.001-100 CSt, for example 0.005-80 CSt, such as 0.01-60 CSt, e.g. 0.05-40 CSt, such as 0.05-20 CSt, for example 0.1-10 CSt, such as 0.5-5 CSt, for example 1.0-3 CSt.

71. A fluid composition according to any one of the preceding claims, wherein the content of said water in said fluid composition is less than 90, 85, 80, 75, 70, 65, 60, 55, 50, 45, 40, 35, 30, 25, 20, 15, 12, 10, 8, 7, 6, 5, 4, 3, 2, 1, 0.5% (w/w) such as in the range of 2-80% (w/w), such as 4-78% (w/w), e.g. 6-76% (w/w), such as 8-74% (w/w), e.g. 10-72% (w/w), such as 12-70% (w/w), e.g. 14-68% (w/w), such as 16-66% (w/w), for example 18-64% (w/w), such as 20-62% (w/w), e.g. 22-60% (w/w), for example 24-58% (w/w), such as 26-56% (w/w), such as 28-54% (w/w), such as 30-52% (w/w), 32-50% (w/w), e.g. 34-48% (w/w), such as 36-46% (w/w), such as 38-44% (w/w), for example 40-42% (w/w).

72. A fluid composition according to any one of the preceding claims, wherein the ratio lignin component:water is selected from the range of 0.4-8.0, such as 0.5-7.9, e.g. 0.6-7.8, such as 0.7-7.6, for example 0.8-7.5, for example 0.9-7.4, such as 1.0-7.3, for example 1.1-7.2, e.g. 1.2-7.1, such as 1.3-7.0, for example 1.4-6.9, such as 1.5-6.8, such as 1.6-6.7, such as 1.7-6.6, for example 1.8-6.5, for example 1.9-6.4, such as 2.0-6.3, for example 2.1-6.2, e.g. 2.2-6.1, such as 2.3-6.0, for example 2.4-5.9, such as 2.5-5.8, such as 2.6-5.7, such as for example 2.8-5.5, for example 2.9-5.4, such as 3.0-5.3, for example 3.1-5.2, e.g. 3.2-5.1, such as 3.3-5.0, for example 3.4-4.9, such as 3.5-4.8, such as 3.6-4.7, such as 3.7-4.6, for example 3.8-4.5, for example 3.9-4.4, such as 4.0-4.3, for example 4.1-4.2, all ratios being based on dry matter content of said lignin component.

73. A fluid composition according to any one of the preceding claims, wherein said fluid composition at 25, 50 or 75° C. is having a viscosity of 20-10,000 CSt, such as 50-8,000 CSt, for example 100-6,000 CSt, such as 200-4,000 CSt, such as 400-2,000 CSt, e.g. 500-1,000 CSt, such as 600-800 CSt.

74. A fluid composition according to any one of the preceding claims, wherein said fluid composition at 25, 50 or 75° C. is having a viscosity of 5-2,000 CSt, such as 10-1,000 CSt, for example 20-800 CSt, such as 50-600 CSt, e.g. 100-400 CSt, such as 200-300 CSt.

75. A fluid composition according to any one of the preceding claims, wherein said fluid composition at 25, 50 or 75° C. is having a viscosity of 2-200 CSt, such as 5-150 CSt, e.g. 10-120 CSt, such as 20-100 CSt, for example 30-80 CSt, such as 40-60 CSt.

76. A fluid composition according to any one of the preceding claims, wherein said fluid composition is having a lower heating value of 4-37 MJ/kg, such as 5-36 MJ/kg, for example 6-35 MJ/kg, such as 7-34 MJ/kg, for example 8-33 MJ/kg, e.g. 9-32 MJ/kg, such as 10-31 MJ/kg, for example 11-30 MJ/kg, such as 12-29 MJ/kg, e.g. 13-28 MJ/kg, such as 14-27 MJ/kg, such as 15-26 MJ/kg, for example 16-25 MJ/kg, such as 17-24 MJ/kg, for example 18-23 MJ/kg, e.g. 19-22 MJ/kg, such as 20-21 MJ/kg.

77. A fluid composition according to any one of the preceding claims, wherein said fluid composition is stable and/or pumpable for 2 weeks or more, such as 3 weeks or more, e.g. 4 weeks or more, such as 6 weeks or more, for example 7 weeks or more, such as 8 weeks or more, such as 2 months or more, e.g. 3 months or more, for example 4 months or more, such as 5 months or more, or 6 months or more; in the sense that no more than 5.0, 4.0, 3.0, 2.0, 1.0 or 0.5% (w/w) of any one of the fractions (e.g. water and or lignin component fraction) of said fluid composition will separate after said specified period of time.

78. A fluid composition according to any one of the preceding claims, wherein gentle stirring, agitation, and/or re-circulation is required for maintaining said stability and/or pumpability.

79. A fluid composition according to any one of the preceding claims, wherein the sulfur content of said fluid composition is 3.0% (w/w) or less, for example 2.8% (w/w) or less, e.g. 2.6% (w/w) or less, for example 2.4% (w/w) or less, e.g. 2.2% (w/w) or less, such as 2.0% (w/w) or less, for example 1.8% (w/w) or less, such as 1.6% (w/w) or less, for example 1.4% (w/w) or less, e.g. 1.2% (w/w) or less, such as 1.0% (w/w) or less, for example 0.8% (w/w) or less, such as 0.4% (w/w) or less, such as 0.2% (w/w) or less, for example 0.1% (w/w) or less, such as 0.08% (w/w) or less, e.g. 0.06% (w/w) or less, such as 0.04% (w/w) or less.

80. A process for the manufacture of a fluid composition according to any one of the preceding claims, said process comprising the steps of:
i. providing a fraction, preferably a solid fraction comprising a lignin component;
ii. providing an organic compound to make up at least part of said liquid organic fraction;
iii. intermixing the fraction provided in step (i) with the organic compound and/or liquid organic fraction provided in step (ii).

81. A process according to claim 80, wherein said lignin component originates from a lignocellulosic biomass which has been subjected to a hydrothermal pretreatment followed by a hydrolysis of at least part of the cellulose and at least part of said hemicellulose present in said lignocellulosic biomass, optionally followed by fermentation and/or hydrolysis.

82. A process according to claim 80 or 81, wherein said lignin component is obtained by pressing a fibrous fraction obtained after subjecting said lignocellulosic biomass to said hydrothermal pretreatment followed by said hydrolysis.

83. A process according to claim 82, wherein said pressing of said fibrous fraction is preceded by rinsing and/or washing of said fibrous fraction.

84. A process according to claim 82 or 83, wherein said lignin component is obtained by mechanically comminuting said pressed fibrous fraction to a desired extent.

85. A process according to any one of the claims 80-84, wherein said lignin component is having characteristics as defined in any of the preceding claims.

86. A process according to any one of the claims 80-85, wherein said organic substance of said liquid fraction is having characteristics as defined in any of the preceding claims.

87. A process according to any one of the claims 80-86, further comprising admixing of an amount of water.

88. A process according to one of the claims 80-87, further comprising admixing of one or more further agent.

89. A process according to claim 88, wherein the further agent is selected from the group comprising or consisting of one or more dispersing agent(s), surfactant(s), hydrotropic agent(s), emulsifier(s), preserving agent(s), and any combination thereof.

90. A process according to any one of the claims 80-89, wherein said intermixing is performed using one or more mixing device(s), such as a mechanical stirrer, high shear mixer, and/or a pump.

91. A process according to any of the claims 80-90, comprising the step of separately intermixing an amount of water and optionally one or more further agent(s) such as a dispersing agent and with (a) said lignin component, (b) said organic compound of said liquid organic fraction, and/or (c) said liquid organic fraction, and optionally wherein said separately mixed mixtures are mixed and stirred.

92. A process for treatment of a lignocellulosic biomass, said process comprising:
a) subjecting said lignocellulosic biomass for hydrothermal pretreatment resulting in a hydrothermally pretreated lignocellulosic biomass;
b) subjecting at least part of said hydrothermally pretreated lignocellulosic biomass obtained in step (a) to a hydrolysis resulting in a liquid fraction comprising soluble carbohydrates, and a fiber fraction comprising a lignin component;
c) optionally subjecting at least part of the liquid fraction obtained in step (b) to a fermentation in order to ferment at least part of said soluble carbohydrates to a fermentation product, such as ethanol, methane or butanol, thereby obtaining a fermentation broth;
d) optionally isolating at least part of said fermentation product from the fermentation broth obtained in step (c) e.g. by distillation;
e) isolating at least part of the lignin component from one or more of: the fiber fraction obtained in step (b); the fermentation broth obtained in step (c); or after isolation of at least a part of the fermentation product in step (d);
f) converting at least part of the lignin component obtained in step (e) to a fluid composition by admixing said lignin component with a liquid organic fraction comprising an organic compound or substance.

93. A process according to claim 92, wherein the fluid composition obtained in step (f) is a fluid composition according to any one of the preceding claims.

94. A process according to claim 92 or 93, wherein said at least part of said lignin fraction is isolated from the fiber fraction obtained in step (b).

95. A process according to any one of the claims 92 to 94, wherein said at least part of said lignin fraction is isolated from said fermentation broth obtained in step (c).

96. A process according to any one of the claims 92 to 95, wherein said lignin component is obtained in step (e) by removing an associated liquid phase by using one or more separation device(s), such as a hydraulic press, a vacuum filtration unit, a belt filter, a rotary filter or a centrifuge decanter.

97. A process according to any one of the claims 92 to 96, wherein said lignin component obtained in step (e) is dried to a residual water content at 110° C. of 2-20% (w/w), such as 4-18% (w/w), for example 6-16% (w/w), such as 8-14% (w/w), e.g. 10-12% (w/w).

98. A process according to any one of the claims 92 to 97, wherein said hydrothermal pretreatment of said lignocellulosic biomass is performed at a temperature of 150-260° C., such as 160-250° C., e.g. 170-240° C., such as 180-230° C., for example 190-220° C., such as 200-210° C.

99. A process according to any of the claims 92 to 98, wherein said hydrothermal pretreatment of said lignocellulosic biomass is performed in a period of residence time of 2-120 min., such as 5-110 min., e.g. 10-100 min., for example 15-90 min., such as 20-80 min., such as 25-70 min., e.g. 30-60 min, such as 35-50 min, such as 40-45 min.

100. A process according to any one of the claims 92 to 99, wherein said hydrothermal pretreatment of said lignocellulosic biomass is performed by subjecting said lignocellulosic biomass to a log severity, log(Rₒ) of 2.5 or more, such as a log(Rₒ) of 2.6 or more, e.g. a log(Rₒ) of 2.7 or more, such as a log(Rₒ) of 2.8 or more, for example a log(Rₒ) of 2.9 or more, such as a log(Rₒ) of 3.0 or more, such as a log(Rₒ) of 3.1 or more, for example a log(Rₒ) of 3.2 or more, e.g. a log(Rₒ) of 3.3 or more, such as a log(Rₒ) of 3.4 or more, such as a log(Rₒ) of 3.5 or more; such as a log(Rₒ) of 3.6 or more; for example such as a log(Rₒ) of 3.7 or more, e.g. a log(Rₒ) of 3.8 or more, for example a log(Rₒ) of 3.9 or more, for example a log(Rₒ) of 4.0 or more, such as a log(Rₒ) of 4.1 or more, or a log(Rₒ) of 4.2 or more; wherein the log severity is defined as: log(Rₒ)=(residence time)×(exp[Temperature−100/14.75]).

101. A process according to any one of the claims 92 to 100, wherein said hydrolysis is an acid catalyzed hydrolysis and/or enzymatic hydrolysis.

102. A process according to claim 101, wherein said hydrolysis is performed by one or more cellulases.

103. A process according to claim 102, wherein said one or more cellulases are selected from the group comprising exo-glucanases, endo-glucanases, hemi-cellulases and beta-glucosidases.

104. A process according to any one of the claims 92 to 103 wherein said hydrolysis is performed for a period of time of 1-200 hours, such as 5-190 hours, such as 10-185 hours, e.g. 15-180 hours, for example 20-175 hours, such as 25-170 hours, such as 30-165 hours, e.g. 35-160 hours, for example 40-155 hours, such as 45-150 hours, such as 50-145 hours, e.g. 55-140 hours, for example 60-135 hours, such as 65-130 hours, such as 70-125 hours, e.g. 75-120 hours, for example 80-115 hours, such as 85-110 hours, such as 90-105 hours, e.g. 95-100 hours.

105. A process according to any one of the claims 92 to 104, wherein said step (b) and step (c) are performed as a separate hydrolysis and fermentation step (SHF), and wherein said hydrolysis is performed at a temperature of 30-72° C., such as 32-70° C., e.g. 34-68° C., for example 36-66° C., such as 38-64° C., e.g. 40-62° C., 42-60° C., e.g. 44-58° C., for example 46-56° C., such as 48-54° C., e.g. 50-52° C.

106. A process according to claim 105, wherein said hydrolysis is performed in a period of time of 70-125 hours, e.g. 75-120 hours, for example 80-115 hours, such as 85-110 hours, such as 90-105 hours, e.g. 95-100 hours.

107. A process according to any one of the claims 92 to 106, wherein said step (b) and step (c) are performed as a simultaneous saccharification and fermentation step (SSF), and wherein said hydrolysis is performed at a temperature of 30-72° C., such as 32-70° C., e.g. 34-68° C., for example 36-66° C., such as 38-64° C., e.g. 40-62° C., 42-60° C., e.g. 44-58° C., for example 46-56° C., such as 48-54° C., e.g. 50-52° C.

108. A process according to claim 107, wherein said hydrolysis is performed in a period of time of 1-12 hours, such as 2-11 hours, for example 3-10 hours, such as 4-9 hours, e.g. 5-8 hours, such as 6-7 hours.

109. A process according to any one of the claims 92 to 108, wherein said step (b) and step (c) are performed as a simultaneous saccharification and fermentation step (SSF), and wherein said fermentation is performed at a temperature of 25-40° C., such as 26-39° C., e.g. 27-38° C., for example 28-37° C., e.g. 29-36° C., for example 30-35° C., such as 31-34° C. or 32-33° C.

110. A process according to claims 92 to 109 wherein said fermentation is performed in a period of time of 100-200 hours, such as 105-190 hours, such as 110-185 hours, e.g. 115-180 hours, for example 120-175 hours, such as 125-170 hours, such as 130-165 hours, e.g. 135-160 hours, for example 140-155 hours, such as 145-150 hours.

111. A process according to any one of the claims 92 to 110, wherein said lignin fraction obtained in step e) is converted to a fluid composition by admixing said lignin fraction with an organic substance, said organic substance constituting a liquid fraction.

112. A process according to any one of the claims 92 to 111, wherein said lignin fraction obtained in step e) is converted to a fluid composition by admixing said lignin fraction with an organic substance and with water, said organic substance constituting a liquid fraction

113. A process according to any of the claims 92 to 112, wherein said lignin fraction obtained in step e) is converted to a fluid composition by admixing said lignin fraction with an organic substance, with water, and with a dispersing agent, said organic substance constituting a liquid fraction.

114. Use of a fluid composition according to any of claims 1-79, or a fluid composition provided by a process according to any one of claims 80-113 to as a fuel.

115. Use according to claim 114, wherein the fluid composition is used as a fuel for a household burner.

116. Use according to claim 114, wherein the fluid composition is used as a fuel for a boiler in a district heat plant or in a combined heat and power (CHP) plant.

117. Use according to claim 115 or 116, wherein the fluid composition is used as a fuel for producing steam or other thermal energy products in an industry or factory using such steam or other thermal energy products to power its power consuming facilities.

118. Use according to any one of claim 114, 116, or 117, wherein the fluid composition is used as a fuel in a boiler in a power plant.

119. Use according to claim any one of claims 114, 116-118, wherein the fluid composition is used as a fuel in a start-up situation in a boiler in a power plant.

120. Use of lignin or a lignin component for a fluid composition according to any one of claims 1 to 113, and optionally, wherein said lignin or a lignin component is dry, i.e. having a residual water content of or around 20% (w/w) or below, such as of or around 15% (w/w), such as of or around 10% (w/w), such as of or around 5% (w/w), such as of or around 2.5% (w/w), or lower.

121. Use according to claim 120, wherein said solid lignin component originates from a lignocellulosic biomass which has been subjected to a hydrothermal pretreatment followed by a hydrolysis.

122. Use according to claim 120 or 121, wherein said lignin component originates from a lignocellulosic biomass which has been subjected to a hydrothermal pretreatment followed by fermentation and/or distillation.

123. Use according to claim 121 or 122, wherein said hydrolysis is an acid catalyzed hydrolysis.

124. Use according to claim 121 or 122, wherein said hydrolysis is an enzymatic hydrolysis.

125. Use according to claim 121 or 122, wherein said hydrolysis comprises acid and enzymatic hydrolysis.

126. Use according to any one of claims 119-125, wherein said fluid composition comprises a solid fraction and a liquid fraction.

127. Use according to claim 126, wherein said solid fraction and said liquid fraction are present in a state of being intermixed; said solid fraction comprises said lignin component; and said liquid fraction comprises an organic substance.

128. Use according to any of the claims 119-127, wherein said fluid composition is a fluid composition according to any of the claims 1-113.

129. Use of a fluid composition according to any of the claims 1-113 for chemical processing of a lignin component and/or providing a conversion product thereof.

130. Use according to claim 129, wherein said chemical processing involves catalytic processing of said lignin component or a conversion product thereof.

131. Use according to claim 129 or 130, wherein said chemical processing involves non-catalytic processing of said lignin component or a conversion product thereof.

132. Use according to any one of claims 129 to 131, wherein said chemical processing involves acid and/or base reactions of said lignin component or a conversion product thereof.

133. Use according to any one of claims 129 to 132, wherein said chemical processing involves an oxidation reaction of said lignin component or a conversion product thereof.

134. Use according to any one of claims 129 to 133, wherein said chemical processing involves a reduction reaction of said lignin component or a conversion product thereof.

135. Use according to any one of claims 129 to 134, wherein said chemical processing involves a hydrolysis reaction of said lignin component or a conversion product thereof.

136. Use according to any one of claims 129 to 135, wherein said chemical processing involves a pyrolysis of said lignin component or a conversion product thereof.

137. Use according to any one of claims 129 to 136, wherein said chemical processing involves a hydrothermal conversion of said lignin component or a conversion product thereof.

138. Use according to any one of claims 129 to 137, wherein said chemical processing involves a supercritical fluid conversion of said lignin component or a conversion product thereof, such as a conversion involving water or methanol or ethanol at supercritical conditions.

139. Use according to any one of claims 129 to 138, wherein said chemical processing involves hydrogenation of said lignin component or a conversion product thereof.

140. Use according to any one of claims 129 to 139, wherein said chemical processing involves hydrodesulfurization of said lignin component or a conversion product thereof.

141. Use according to any one of claims 129 to 140, wherein said chemical processing involves hydrodenitrogenation of said lignin component or a conversion product thereof.

142. Use according to any one of claims 129 to 141, wherein said chemical processing involves hydrodeoxygenation of said lignin component or a conversion product thereof.

143. Use according to any one of claims 129 to 142, wherein said chemical processing involves hydrocracking of said lignin component or a conversion product thereof.

144. Use according to any one of claims 129 to 143, wherein said chemical processing involves hydrodenitrification of said lignin component or a conversion product thereof.

145. Use according to any one of claims 129 to 144 wherein said chemical processing involves cracking of said lignin component of said lignin component or a conversion product thereof.

146. Use according to claim 145, wherein said cracking is a technical cracking of said lignin component or a conversion product thereof.

147. Use according to claim 145, wherein said cracking is a catalytical cracking of said lignin component or a conversion product thereof.

148. A fluid composition according to any one of claims 1-113, comprising, containing, consisting, or consisting essentially of:
i. “L” (Lignin component) 5-60% (w/w)
ii. “O” (Liquid organic fraction) 0-60% (w/w)
iii. “W” (Water) 0-95% (w/w)
iv. “A” (Further agent) 0-5.0 or 0-1.0% (w/w);

149. A fluid composition according to claim 148, wherein the “L-O-W” expressed in the individual % (w/w) is, or is around:
5-0-95, 5-5-90, 5-10-85, 5-15-80, 5-20-75, 5-25-70, 5-30-65, 5-35-60, 10-0-90, 10-5-85, 10-10-80, 10-15-75, 10-20-70, 10-25-65, 10-30-60, 15-0-85, 15-5-80, 15-10-75, 15-15-70, 15-20-65, 15-25-60, 15-30-55, 20-0-80, 20-5-75, 20-10-70, 20-15-65, 20-20-60, 20-25-55, 20-30-50, 25-0-75, 25-5-70, 25-10-65, 25-15-60, 25-20-55, 25-25-50, 25-30-45, 30-0-70, 30-5-65, 30-10-60, 30-15-55, 30-20-50, 30-25-45, 30-30-40, 35-0-65, 35-5-60, 35-10-55, 35-15-50, 35-20-45, 35-25-40, 35-30-35, 40-0-60, 40-5-55, 40-10-50, 40-15-45, 40-20-40, 40-25-35, 40-30-30, 45-0-55, 45-5-50, 45-10-45, 45-15-40, 45-20-35, 45-25-30, 45-30-25, 50-0-50, 50-5-45, 50-10-40, 50-15-35, 50-20-30, 50-25-25, 50-30-20, 55-0-45, 55-5-40, 55-10-35, 55-15-30, 55-20-25, 55-25-20, 55-30-15, 60-0-40, 60-5-35, 60-10-30, 60-15-25, 60-20-20, 60-25-15, 60-30-10, 10-35-55, 15-35-50, 20-35-45, 25-35-40, 30-35-35, 35-35-30, 40-35-25, 45-35-20, 50-35-15, 55-35-10, 60-35-5, 5-40-55, 10-40-50, 15-40-45, 20-40-40, 25-40-35, 30-40-30, 35-40-25, 40-40-20, 45-40-15, 50-40-10, 55-40-5, 60-40-0, 5-45-50, 10-45-45, 15-45-40, 20-45-35, 25-45-30, 30-45-25, 35-45-20, 40-45-15, 45-45-10, 50-45-5, 55-45-0, 5-50-45, 10-50-40, 15-50-35, 20-50-30, 25-50-25, 30-50-20, 35-50-15, 40-50-10, 45-50-5, 50-50-0, 5-55-40, 10-55-35, 15-55-30, 20-55-25, 25-55-20, 30-55-15, 35-55-10, 40-55-5, 45-55-0, 5-60-35, 10-60-30, 15-60-25, 20-60-20, 25-60-15, 30-60-10, 35-60-5, or 40-60-0.

150. A fluid composition according to claim 148 or 149, wherein the lignin component is, or is around 5, 10, 15, 20, 25, 30, 35, 40, 45, 50, 55, or 60% (w/w).

151. A fluid composition according to any one of claims 148-150, wherein the liquid organic fraction is, or is around 0, 5, 10, 15, 20, 25, 30, 35, 40, 45, 50, 55, or 60% (w/w).

152. A fluid composition according to any one of claims 148-151, wherein the water is, or is around 5, 10, 15, 20, 25, 30, 35, 40, 45, 50, 55, or 60% (w/w).

153. A fluid composition according to any one of claims 149-152, wherein “around” means+/−1, 2, or 2.5% (w/w) based on the total composition.

154. A fluid composition according to any one of claims 148-153, wherein the further agent is or is around 0, 0.05, 0.10, 0.15, 0.20, 0.25, 0.30, 0.40, 0.45, 0.50, 0.55, 0.60, 0.65, 0.70, 0.75, 0.8, 0.85, 0.90, 0.95, or 1.0% (w/w).

155. A fluid composition according to claim 154, wherein “around” means+/−0.1, 0.2, or 0.25% (w/w) based on the total composition.

156. A process according to any one of claims 80-113 comprising the fluid composition according to any one of claims 148-155.

157. A use according to any one of claims 114-147 comprising the fluid composition according to any one of claims 148-155.
